SEQUENCE LISTING (30 sequences)

SEQ ID NO: 1 - protein, 498 aa, Homo sapiens [sequence omitted]

SEQ ID NO: 2 - protein, 501 aa, Mus musculus
MAAAAATAAAAAAPSGGGGGGEEERLEEKSEDQDLQGLRDKPLKFKKAKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVS

SEQ ID NO: 3 - DNA, 1497 bp, Homo sapiens
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga

SEQ ID NO: 4 - DNA, 1506 bp, Mus musculus
atggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctga

SEQ ID NO: 5 - DNA, 278 bp, Artificial Sequence
  chicken beta-actin (CBA) promoter
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 6 - DNA, 223 bp, Mus musculus
ctttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaacca

SEQ ID NO: 7 - DNA, 224 bp, Homo sapiens
ctttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaacca

SEQ ID NO: 8 - DNA, 171 bp, Homo sapiens
ctttacacggagcggattgcaaagcaaaccaacaagaataaaggcagctgttgtctcttctccttatgggtagggctctgacaaagcttcccgattaactgaaataaaaaatatttttttttctttcagtaaaaaaaaaaaaaaaaaaaaaaaaaaaaaaaaaaaaaaaaa

SEQ ID NO: 9 - DNA, 490 bp, Homo sapiens
tcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 10 - protein, 736 aa, Artificial Sequence
  amino acid sequence of the (wild-type) AAV9 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 743 aa, Artificial Sequence
  amino acid sequence of the AAV-PHP.B capsid VP1 protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 743 aa, Artificial Sequence
  amino acid sequence of the AAV-PHP.eB capsid VP1 protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 3649 bp, Artificial Sequence
  CBA-iMECP2 cassette sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgaacgccaccatgggcaagcctatccctaaccctctgctgggcctggactccacaggcagcggcaccggtatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgactttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagatatctctagagatatcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 14 - protein, 7 aa, Artificial Sequence
  sequence from VP1 capsid protein sequence
TLAVPFK

SEQ ID NO: 15 - protein, 7 aa, Artificial Sequence
  sequence from VP1 capsid protein sequence
KFPVALT

SEQ ID NO: 16 - protein, 11 aa, Artificial Sequence
  sequence from VP1 capsid protein sequence
DGTLAVPFKAQ

SEQ ID NO: 17 - DNA, 4045 bp, Artificial Sequence
  vector AAV-CBA-V5-iMecp2-3`pUTR-hGHpA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgaacgccaccatgggcaagcctatccctaaccctctgctgggcctggactccacaggcagcggcaccggtatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgactttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagatatctctagagatatcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 3608 bp, Artificial Sequence
  vector AAV-Mecp2 promoter (1.4kb)-V5-iMecp2-3`pUTR-hGHpA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagtatcgatactcgagatttcaactgctactgtcctggttaaagccttcatcatctatctttcttcaactgctgccaggacctctggaccagccagttcttcattcttcactggcaacataggttttatggtgacagctagtgactcaaatatttatcaagggcttctcatctcaaaataatctcctagttcttttggtggcctaggtctctctccagtcacactggcctccttagtaaggcaggcatagtccttccttagagtgtttaaacttgcctagaatgttttccccaattacccatattgggagacgacatgagggcaaaagctagagggtatcataatagcacttcttttgtccttgccctatctatttcaaagtctttatctctgtgcaaaattttaagttctactttcttgtatgtttagtatgactcttccttaccaggagtctagtttgtctccttgttcagtactaaaacagtgcctagcaaataaatgaatagagaggggagccaaatttgaatcagaaagtctcttgttgcatagtgtttaaaaaacaaacaaagaaagaaagtctcttgttgagcatttgtttagcacaaagagcattggatgctgactggtatcagggtaaggctgctttgacaatgctccctctggcctcactcccttttatacgtacttccatcaaaccatctgattcaacaatgacagaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtcaatcgctagcggatccgttaacgccaccatgggcaagcctatccctaaccctctgctgggcctggactccacaggcagcggcaccggtatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgactttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagatatctctagagtcgacgaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 19 - DNA, 4033 bp, Artificial Sequence
  vector AAV-CBA-V5-iMecp2-3`aUTR-hGHpA (M2c)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgaacgccaccatgggcaagcctatccctaaccctctgctgggcctggactccacaggcagcggcaccggtatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctaatctagaagctcgctgatcagcctcacaagaataaaggcagctgttgtctcttcagaagtagctttgcacttttctaaactaggaatatcaccaggactgttactcaatgtgtgctgcaggaaagcactgatatatttaaaaacaaaaggtgtaacctatttattatataaagagtttgccttataaatttacataaaaatgtccgtttgtgtcttttgttgtaaaaatcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 4143 bp, Artificial Sequence
  vector AAV-CBA-5`UTR-V5-iMecp2-3`pUTR-hGHpA (M2d)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgtcgggagggcggggcgcgacgtctgccgtgcggggtcccggcatcggttgcgcgcgcgctccctcctctcggagagagggctgtggtaaaacccgtccggaaagctagcggatccgaacgccaccatgggcaagcctatccctaaccctctgctgggcctggactccacaggcagcggcaccggtatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgactttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagatatctctagagatatcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 3804 bp, Artificial Sequence
  vector AAV-CBA-V5-iMecp2-hGHpA (M2e)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgaacgccaccatgggcaagcctatccctaaccctctgctgggcctggactccacaggcagcggcaccggtatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgagtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 19 bp, Artificial Sequence
  sequence encoding an shRNA that inhibits expression of MeCP2, shMecp2
gattgtagattcaggttaa

SEQ ID NO: 23 - DNA, 3340 bp, Artificial Sequence
  vector AAV-shMecp2-CBA-GFP
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagagaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatccccgattgtagattcaggttaattcaagagattaacctgaatctacaatctttttggaaaagcttatcgataacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacacggtcgacgaattcgttaacggatccgaacgccaccatgggcaagcctatccctaaccctctgctgggcctggactccacaggcagcggcaccggtggatcctctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctagagatatcctcgagagatcgatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 4339 bp, Artificial Sequence
  vector AAV-shMecp2-iMecp2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagagaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatccccgattgtagattcaggttaattcaagagattaacctgaatctacaatctttttggaaaagcttatcgataacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacacggtcgacgaattcgttaacggatccgaacgccaccatgggcaagcctatccctaaccctctgctgggcctggactccacaggcagcggcaccggtatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgactttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagatatctctagagatatcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 3981 bp, Artificial Sequence
  vector AAV-CBA-iMecp2-3`pUTR-hGHpA (M2f)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgccaccatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgactttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagatatctctagagatatcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 26 - DNA, 20 bp, Artificial Sequence
  sgRNA sequence designed on the third exon of MECP2
aagcttaagcaaaggaaatc

SEQ ID NO: 27 - DNA, 61 bp, Artificial Sequence
  wild-type MECP2 gene exon 3 sequence
acacggaagcttaagcaaaggaaatctggccgctctgctgggaagtatgatgtgtatttga

SEQ ID NO: 28 - protein, 20 aa, Artificial Sequence
  amino acid sequence encoded by wild-type MECP2 gene exon 3
TRKLKQRKSGRSAGKYDVYL

SEQ ID NO: 29 - protein, 19 aa, Artificial Sequence
  MECP2 knock-out (KO) exon 3 amino acid sequence
TRKLKQRNLAALLGSMMCI

SEQ ID NO: 30 - DNA, 60 bp, Artificial Sequence
  MECP2 KO exon 3 sequence (single nucleotide deletion)
acacggaagcttaagcaaaggaatctggccgctctgctgggaagtatgatgtgtatttga

(19) United States
(12) Patent Application Publication
(10) Pub. No.: US 2022/0133912 A1
(43) Pub. Date: May 5, 2022

(54) Gene Therapy

(71) Applicant: Ospedale San Raffaele S.R.I., Milan (IT)

(72) Inventors: Vania Broccoli, Milan (IT); Mirko Luoni, Milan (IT); Serena Giannelli, Milan (IT)

(21) Appl. No.: 17/503,088

(22) Filed: Oct. 15, 2021

Related U.S. Application Data
(63) Continuation of application No. PCT/EP2020/060627, filed on Apr. 15, 2020.

(30) Foreign Application Priority Data
Apr. 15, 2019 (GB) 1905301.6

(51) Int. Cl. A61K 48/00; C07K 14/47; C12N 15/86

(52) U.S. Cl. CPC A61K 48/0066; C07K 14/4702; C12N 15/86; C12N 2840/10; C12N 2740/16043; C12N 2750/14143; C12N 2510/00

(57) ABSTRACT

A polynucleotide comprising a nucleotide sequence encoding methyl-CpG binding-protein 2 (MeCP2) operably linked to a strong promoter and a 3′-UTR, wherein the 3′-UTR is less than or equal to about 1000 bp in length.

### CROSS-REFERENCE TO RELATED APPLICATIONS

This application is a continuation of International Application No. PCT/EP2020/060627, filed on Apr. 15, 2020, now expired, which claims the benefit of priority to United Kingdom Application No. 1905301.6, filed on Apr. 15, 2019, now expired.

### FIELD OF THE INVENTION

The present invention relates to compounds for use in the treatment of neurological diseases. More specifically, the invention relates to polynucleotides comprising a nucleotide sequence encoding methyl-CpG binding-protein 2 (MeCP2) and uses thereof in the treatment of Rett syndrome.

### BACKGROUND TO THE INVENTION

Rett syndrome (RTT) is a severe neurological disorder and a leading cause of intellectual disabilities in girls. RTT is characterised by a period of 6-12 months of overtly normal development followed by rapid regression with the loss of the purposeful motor skills and the onset of repetitive and autistic behaviours.

In the vast majority of cases RTT is caused by loss-of-function mutations in the MECP2 gene, which encodes the methyl-CpG binding protein 2 (MeCP2), a global chromatin regulator highly expressed in neurons (Bienvenu, T. et al. (2006) Nat Rev Genet 7: 415-426). Recent studies have revealed that the MECP2 loss significantly alters neuronal activity leading to a progressive imbalance of the excitatory-inhibitory synaptic activity across the brain with divergent modalities occurring between different circuits and regions of the brain (Banerjee, A. et al. (2016) PNAS 113: E7287-E7296).

One study demonstrated that the RTT pathological phenotype can be significantly reversed in mice by re-activating MECP2 even at advanced disease stages (Guy, J. et al. (2007) Science 315: 1143-1147). In fact, genetic reactivation of MECP2 in more than 70% of the neurons in adult mice normalised brain morphology and significantly improved several sensory-motor dysfunctions (Robinson, L. et al. (2012) Brain 135: 2699-2710).

These findings provide strong evidence that MeCP2 is a key factor in maintaining full neurological function during adulthood. Consistently, multiple pathological manifestations exhibited by adult mutant RTT mice can be fully recapitulated by deleting MeCP2 exclusively in adulthood.

Although MeCP2 is a ubiquitous neural epigenetic factor, its selective inactivation in the GABAergic neurons leads to several RTT distinctive phenotypes, suggesting that key neurological deficits in RTT are mediated by GABAergic neuronal dysfunctions. MECP2 genetic reconstitution exclusively in GABAergic neurons resulted in significant improvement of key motor and cognitive deficits in RTT mice (Ure, K. et al. (2016) eLife 5: 185). Similarly, preferential re-expression of MECP2 in astrocytes consistently ameliorated RTT neurological symptoms in the mutant murine model (Lioy, D. T. et al. (2011) Nature 475: 497-500). Thus, GABAergic neurons and astrocytes may be considered crucial targets for therapeutic strategies.

Despite the proven genetic reversibility of the RTT disease phenotype in mice, translational treatments aiming at curing the disease or some of its neurological symptoms have not been successful to date. In fact, MeCP2 is a global determinant of the neural chromatin structure and is a pervasive regulator of gene expression in brain cells and, thus, it remains challenging to target a single MeCP2 downstream pathway to obtain a substantial therapeutic benefit (Katz, D. M. et al. (2016) Trends in Neurosciences 39: 100-113).

The inherent monogenic nature of RTT makes gene therapy a strong translational option for this disease. However, MECP2 gene duplication in humans is responsible for a serious and clinically distinguished neurodevelopmental disorder. Affected males present with early hypotonia, limb spasticity and severe intellectual disability. Thus, a successful gene therapy for RTT may require delivery of MeCP2 in a range comparable with endogenous levels.

Recent studies have suggested that intravenous administration of an AAV9 expressing wild-type (WT) MECP2 attenuated neurological dysfunctions and extended lifespan in RTT mice (Matagne, V. et al. (2017) Neurobiology of Disease 99: 1-11). However, the limited brain transduction obtained in these studies was not sufficient to determine a correlation between the viral dose, transduction efficiency and therapeutic benefits. Based on those studies, it was unclear whether a gene therapy approach is capable of rescuing molecular dysfunctions and transcriptional alterations caused by loss of MECP2 in the adult brain.

Accordingly, there remains a significant need for improved approaches for treating Rett syndrome.

### SUMMARY OF THE INVENTION

The inventors have developed an instability-prone MeCP2 (iMecp2) transgene cassette, which prevents supraphysiological MeCP2 protein levels in transduced neural tissues. While not wishing to be bound by theory, the inventors believe that this may be achieved by increasing RNA destabilisation and inefficient protein translation of the MeCP2 transgene.

The inventors have demonstrated that Intravenous injections of an iMeCP2-encoding vector (e.g an AAV9 PHP.eB vector) in symptomatic male and female MeCP2 mutant mice models significantly ameliorated the disease progression with improved locomotor activity, coordination, lifespan and normalisation of altered gene expression and mTOR signalling.

The inventors also surprisingly demonstrated that iMecp2 administration did not result in severe toxicity effects either in female MeCP2 mutant or in wild-type animals.

Overall, delivery of the iMeCP2 cassette, such as AAV9 PHP.eB-mediated delivery, provided widespread and efficient gene transfer maintaining physiological MeCP2 protein levels in the brain, providing a system with significant therapeutic efficacy and increased safety in treating Rett syndrome.

In one aspect the invention provides a polynucleotide comprising a nucleotide sequence encoding methyl-CpG binding-protein 2 (MeCP2) operably linked to a strong promoter and a 3′-UTR, wherein the 3′-UTR is less than or equal to about 1000 bp in length.

In some embodiments, the nucleotide sequence encoding MeCP2 comprises a sequence selected from the group consisting of:
(a) a nucleotide sequence encoding an amino acid sequence that has at least 70% identity to SEQ ID NO: 1 or 2;
(b) a nucleotide sequence that has at least 70% identity to SEQ ID NO: 3 or 4; and
(c) the nucleotide sequence of SEQ ID NO: 3 or 4.

In some embodiments, the promoter is a neuron-, glial- or astrocyte-specific strong promoter.

In some embodiments, the promoter is selected from the group consisting of a chicken β-actin (CBA) promoter, a β-actin promoter, a CAG promoter, a cytomegalovirus (CMV) promoter, a human elongation factor-1-alpha (HEF-1-alpha), a Chinese hamster elongation factor-1-alpha (CHEF-1-alpha) promoter and a phosphoglycerate kinase (PGK) promoter.

In preferred embodiments, the promoter is a chicken β-actin (CBA) promoter.

In some embodiments, the 3′-UTR is less than or equal to about 900 bp, 800 bp, 700 bp, 600 bp, 500 bp, 400 bp, 300 bp or 200 bp in length.

In some embodiments, the 3′-UTR is less than or equal to about 500 bp in length. In preferred embodiments, the 3′-UTR is less than or equal to about 250 bp in length.

In some embodiments, the 3′-UTR is about 50-1000 bp, 50-900 bp, 50-800 bp, 50-700 bp, 50-600 bp, 50-500 bp, 50-400 bp, 50-300 bp or 50-300 bp in length.

In some embodiments, the 3′-UTR is about 50-300 bp in length. In some embodiments, the 3′-UTR is about 50-250 bp in length. In some embodiments, the 3′-UTR is about 50-200 bp in length.

In some embodiments, the 3′-UTR is about 100-300 bp in length. In some embodiments, the 3′-UTR is about 100-250 bp in length. In some embodiments, the 3′-UTR is about 100-200 bp in length.

In some embodiments, the 3′-UTR is about 150-300 bp in length. In preferred embodiments, the 3′-UTR is about 150-250 bp in length.

In preferred embodiments, the 3′ UTR is derived from the MeCP2 3′-UTR. In preferred embodiments, the 3′-UTR is a truncated MeCP2 3′UTR.

In some embodiments, the 3′-UTR is a MeCP2 3′-UTR of less than or equal to about 900 bp, 800 bp, 700 bp, 600 bp, 500 bp, 400 bp, 300 bp or 200 bp in length.

In some embodiments, the 3′-UTR is a MeCP2 3′-UTR of less than or equal to about 500 bp in length. In preferred embodiments, the 3′-UTR is a MeCP2 3′-UTR of less than or equal to about 250 bp in length.

In some embodiments, the 3′-UTR is a MeCP2 3′-UTR of about 50-1000 bp, 50-900 bp, 50-800 bp, 50-700 bp, 50-600 bp, 50-500 bp, 50-400 bp, 50-300 bp or 50-300 bp in length.

In some embodiments, the 3′-UTR is a MeCP2 3′-UTR of about 50-300 bp in length. In some embodiments, the 3′-UTR is a MeCP2 3′-UTR of about 50-250 bp in length. In some embodiments, the 3′-UTR is a MeCP2 3′-UTR of about 50-200 bp in length.

In some embodiments, the 3′-UTR is a MeCP2 3′-UTR of about 100-300 bp in length. In some embodiments, the 3′-UTR is a MeCP2 3′-UTR of about 100-250 bp in length. In some embodiments, the 3′-UTR is a MeCP2 3′-UTR of about 100-200 bp in length.

In some embodiments, the 3′-UTR is a MeCP2 3′-UTR of about 150-300 bp in length. In preferred embodiments, the 3′-UTR is a MeCP2 3′-UTR of about 150-250 bp in length.

In some embodiments, the polynucleotide further comprises a polyadenylation sequence operably linked to the nucleotide sequence encoding MeCP2.

In some embodiments, the polynucleotide further comprises a nucleotide sequence encoding a tag (e.g. a V5 tag). In some embodiments, the polynucleotide does not comprise a nucleotide sequence encoding a tag (e.g. a V5 tag).

In some embodiments, the polynucleotide comprises or consists of a nucleotide sequence that has at least 70%, 80%, 90%, 95%, 96%, 97%, 98% 99% or 100% identity to SEQ ID NO: 13.

In some embodiments, the polynucleotide comprises or consists of a nucleotide sequence that has at least 70%, 80%, 90%, 95%, 96%, 97%, 98% 99% or 100% identity to SEQ ID NO: 17.

In some embodiments, the polynucleotide comprises or consists of a nucleotide sequence that has at least 70%, 80%, 90%, 95%, 96%, 97%, 98% 99% or 100% identity to SEQ ID NO: 19.

In some embodiments, the polynucleotide comprises or consists of a nucleotide sequence that has at least 70%, 80%, 90%, 95%, 96%, 97%, 98% 99% or 100% identity to SEQ ID NO: 20.

In some embodiments, the polynucleotide does not comprise a sequence encoding a V5 tag. For example, the invention may contemplate sequences that are the same as the sequences disclosed herein, but with the proviso that a sequence encoding a V5 tag is deleted.

In some embodiments, the polynucleotide comprises or consists of a nucleotide sequence that has at least 70%, 80%, 90%, 95%, 96%, 97%, 98% 99% or 100% identity to SEQ ID NO: 25.

In some embodiments, the polynucleotide further comprises a nucleotide sequence encoding an inhibitor of MeCP2 expression.

In some embodiments, the inhibitor is an shRNA, siRNA, miRNA or antisense DNA/RNA. Preferably, the inhibitor is an shRNA.

In another aspect the invention provides a vector comprising the polynucleotide of the invention.

In some embodiments, the vector is a viral vector.

In some embodiments, the vector is an AAV, retroviral, lentiviral or adenoviral vector. In preferred embodiments, the vector is an AAV vector.

In some embodiments, the vector is in the form of a viral vector particle. In preferred embodiments, the vector is in the form of an AAV vector particle.

In preferred embodiments, the viral vector particle is adapted for crossing the blood-brain barrier. In preferred embodiments, the AAV vector particle is adapted for crossing the blood-brain barrier.

In some embodiments, the AAV vector particle comprises an artificial capsid amino acid sequence. In some embodiments, the artificial capsid amino acid sequence enables the vector particle to cross the blood-brain barrier.

In some embodiments, the AAV vector particle comprises a VP1 capsid protein comprising an amino acid sequence comprising at least four contiguous amino acids, such as at least five or 6, preferably seven amino acids, from the sequence TLAVPFK (SEQ ID NO: 14) or KFPVALT (SEQ ID NO: 15).

In some embodiments, the AAV vector particle comprises a VP1 capsid protein comprising an amino acid sequence comprising the sequence DGTLAVPFKAQ (SEQ ID NO: 16).

In some embodiments, the AAV vector particle comprises a capsid comprising an amino acid sequence that has at least 70%, 75%, 80%, 85% or 90% identity to SEQ ID NO: 11, more preferably at least 95%, 96%, 97%, 98%, 99% or 100% identity to SEQ ID NO: 11.

In some embodiments, the AAV vector particle comprises a capsid comprising an amino acid sequence that has at least 70%, 75%, 80%, 85% or 90% identity to SEQ ID NO: 12, more preferably at least 95%, 96%, 97%, 98%, 99% or 100% identity to SEQ ID NO: 12.

In some embodiments, the AAV vector particle has a serotype selected from the group consisting of AAV9; AAV9 PHP.B; AAV9 PHP.eB; and AAVrh10. In preferred embodiments, the AAV vector particle has a AAV9 PHP.eB serotype.

In some embodiments, the AAV vector particle comprises a capsid selected from the group consisting of an AAV9; AAV9 PHP.B; AAV9 PHP.eB; and AAVrh10 capsid. In preferred embodiments, the AAV vector particle comprises a AAV9 PHP.eB capsid.

In another aspect the invention provides a cell comprising the polynucleotide or vector of the invention.

In another aspect the invention provides a pharmaceutical composition comprising the polynucleotide, vector or cell of the invention and a pharmaceutically-acceptable carrier, diluent or excipient.

In some embodiments, the pharmaceutical composition is formulated for systemic or local delivery.

In some embodiments, the pharmaceutical composition is formulated for intravascular, intravenous, intra-arterial, intracranial or intraparenchymal brain delivery.

In another aspect the invention provides the polynucleotide, vector or cell of the invention for use in medicine.

In another aspect the invention provides the polynucleotide, vector or cell of the invention for use in treating or preventing Rett syndrome.

In another aspect the invention provides a method for treating or preventing Rett syndrome comprising administering the polynucleotide, vector or cell of the invention to a subject in need thereof.

In some embodiments, the polynucleotide, vector or cell is administered to a subject systemically or locally.

In some embodiments, the polynucleotide, vector or cell is administered to a subject intracranially or intraparenchymally.

In preferred embodiments, the polynucleotide, vector or cell is administered to a subject intravenously.

In some embodiments, the polynucleotide, vector or cell is administered simultaneously, sequentially or separately in combination with an immunosuppressant.

In some embodiments, the immunosuppressant is cyclosporin A (CsA).

In some embodiments, the vector is administered at a dosage of 10⁸ to 10¹² vg/20 g, preferably 10⁹ to 10¹¹ vg/20 g. In preferred embodiments, the vector is administered at a dosage of 10¹⁰ to 10¹¹ vg/20 g.

In some embodiments, the vector is administered at a dosage of 5×10⁹ to 5×10¹⁴ vg per kg. In some embodiments, the vector is administered at a dosage of 5×10¹⁰ to 5×10¹³ vg per kg. In preferred embodiments, the vector is administered at a dosage of 5×10¹¹ to 5×10¹² vg per kg.

In another aspect, the invention provides a polynucleotide comprising a nucleotide sequence encoding methyl-CpG binding-protein 2 (MeCP2) operably linked to a strong promoter, as disclosed herein with the proviso that the polynucleotide does not comprise a 3′-UTR.

### DESCRIPTION OF THE DRAWINGS

FIG. 1

RNA stability and translational efficiency of the viral Mecp2 transgene. (a) Illustration of the AAV vectors expressing V5-tagged Mecp2 or GFP under the control of Chicken β-Actin (CBA) promoter or Mecp2 core (M2) promoter. Vectors include murine Mecp2 coding sequence with a synthetic 3′UTR sequence (3′aUTR, M2c); both 3′pUTR and 5′UTR (M2d); no UTR (M2e); or 3′pUTR and no V5 tag (M2f). (b) Western-blot analysis for V5, MeCP2 and Actin protein levels in GFP infected (control, CBA-GFP) WT neurons and Mecp2^{-/y} neurons infected with CBA-Mecp2 and M2-Mecp2. Quantification was performed using densitometric analysis of MeCP2 and V5 relative to Actin signal and expressed in arbitrary units (n=3) (c) Immunostaining of Mecp2^{-/y} (control untreated and infected with M2-Mecp2 or CBA-Mecp2) and wild-type neurons for MeCP2 and TUBB3. (d) qRT-PCR quantification of viral Mecp2 DNA copies in Mecp2^{-/y} neurons relative to wild-type Mecp2 DNA (n=3). (e) Illustration of the experimental work-flow to study the RNA stability. (f-h) RNA stability of total (e) viral (f) and endogenous (g) Mecp2 determined by qRT-PCRs (n=3). (i) Illustration of the experimental work-flow to study translational efficiency. (j) qRT-PCR of viral and endogenous Mecp2 RNA in the ribosomal fraction normalized on the total RNA in WT and Mecp2^{-/y} neurons (n=4 endogenous Mecp2, n=4 exogenous Mecp2 in Mecp2^{-/y} neurons, n=3 exogenous Mecp2 in WT neurons. Error bars, Standard Deviation (SD). ** p<0.01, *** p<0.001, compared groups are indicated by black lines. ANOVA-one way, (b, j, f, g, h and Tukey's post hoc test. Scale bar: 100 μm (c). (p) RNA stability of endogenous (in WT neurons) and viral (from the 5 different vectors described, in KO neurons) Mecp2 transcript determined by qRT-PCRs (n=3, t=300 min were normalized over t=0 values and compared among different treatments).

Stability of the viral human iMECP2 construct in iPSC-derived cortical neurons. (k) Illustration of the CRISPR-Cas9 based strategy for genetic editing of male control human iPSCs to obtain MECP2 KO cells. The sgRNA selected for this approach annealed on exon 3 of the MECP2 gene and generated a single nucleotide deletion. That resulted in a frameshift of its coding sequence and a premature STOP codon 12 residues downstream. (I) Human iPSCs carrying this mutation (MECP2 KO) maintained their pluripotency marker Oct4 but did not present detectable MeCP2 protein as tested by immunofluorescence when compared to control cells (Ctrl). (m) WT and MECP2^{-/y} iPSCs were successfully differentiated into neurons (MAP2 staining) and transduced using PHP.eB vectors carrying either GFP or iMECP2.

Immunofluorescence respectively for GFP and MeCP2 attested neuronal transduction. (n) Schematics of the AAV vector used for RNA stability experiment and expressing V5-tagged MECP2 e2 under the control of Chicken β-Actin (CBA) promoter. (o) MECP2 RNA stability was determined by qRT-PCRs in WT neurons, to test the endogenous transcript (blue bars, n=3), and in KO neurons infected with the aforementioned vector, to test the viral transcript (yellow bars n=3). t=300 min were normalized over t=0 values and compared among different treatments. UT: untreated.

FIG. 2

PHP.eB-mediated iMecp2 gene transfer in symptomatic Mecp2^{-/y} mouse brains. (a) Illustration of the experimental setting to restore the expression of MeCP2 in symptomatic Mecp2^{/y} animals by means of AAV-PHP.eB. (b) Low magnification of MeCP2 immunostaining in brains of Mecp2^{-/y} control untreated (UT) and treated animals (1*10¹⁰, 1*10¹¹, 1*10¹² vg/mouse). (c) High magnification immunostaining for MeCP2 and V5 in cortex and striatum derived from wild-type (WT) and MeCP2 treated Mecp2^{-/y} (1*10⁹, 1*10¹⁰, 1*10¹¹, 1*10¹² vg/mouse) animals (Mecp2-KO). Nuclei were stained with DAPI (merge panels). Bottom panel: bar graphs showing the fraction of MeCP2 positive on the total DAPI positive (n=4 for 1*10⁹-1*10¹⁰-1*10¹²; n=8 1*10¹¹ vg/mouse). (d) Western blot analysis to quantify MeCP2 over Actin protein levels in cortex (upper panel) and striatum (lower panel) derived from WT, untreated Mecp2^{-/y} (KO) and iMecp2 treated Mecp2^{-/y} (1*10⁹, 1*10¹⁰, 1*10¹¹, 1*10¹² vg/mouse) animals and corresponding densitometric quantification expressed in arbitrary units (n=4 for 1*10⁹-1*10¹⁰-1*10¹²; n=8 1*10¹¹ vg/mouse). Error bars, SD. * p<0.05, ** p<0.01 and *** p<0.001 as compared to WT mice (ANOVA-one way with Tukey's post hoc test). Scale bars: 500 μm (b), 20 μm (c).

FIG. 3

Behavioural rescue of symptomatic Mecp2^{-/y} animals after PHP.eB-iMecp2 treatment. (a) Kaplan-Meier survival plot for Mecp2^{-/y} mice injected with different doses (1*10⁹ [n=6], 1*10¹⁰ [n=6], 1*10¹¹ [n=10], 1*10¹² [n=6] vg/mouse) of PHP.eB-iMecp2 compared to Mecp2^{-/y} treated with PHP.eB-GFP (KO-GFP, n=10) and WT (n=14) animals. Mice treated with 1*10¹¹ vg/mouse dosage had a median survival period significantly longer than that of vehicle-treated controls (** p<0.01, Mantel-Cox test). (b) Mouse body weight was monitored every two weeks and represented as mean of each group (p<0.05 versus KO-GFP in 1*10¹⁰ [5^{th}-10^{th} week, wk] and in 1*10¹¹ [7^{th}-9^{th} wk]). (c) Spontaneous locomotor activity was tested in a spontaneous field arena and shown as representative traces and quantification of total distance. (** p<0.01 and *** p<0.001 as compared to WT mice). (d) Representative pictures of animals WT, KO and KO treated with most efficacious dose (1*10¹¹) indicate the absence of hindlimb clasping in KO treated animals as far as 14 weeks of age. All groups of animals were tested for motor coordination using beam balance test and quantified as crossing time (e, p<0.05 versus KO-GFP in 1*10¹⁰[7^{th}-10^{th} wk] and in 1*10¹¹ [7^{th}-9^{th} wk]) and number of errors (f, p<0.05 versus KO-GFP in 1*10¹⁰ [8^{th}-10^{th} wk] and in 1*10¹¹[7^{th}-10^{th} wk]). (g) General phenotypic assessment evaluated through the aggregate severity score (p<0.05 versus KO-GFP in 1*10¹⁰[7^{th}-10^{th} wk] and in 1*10¹¹ [6^{th}-10^{th} wk]). Error bars, SD. ANOVA-two way (b, e, f, g) or ANOVA-one way (c) with Tukey's post hoc test.

FIG. 4

iMecp2 elicits a strong immune response in Mecp2^{-/y} but not wild-type mice. (a) 7 out of 10 Mecp2^{/y} mice injected with a 1*10¹¹ dose of PHP.eB-iMecp2 presented with exudative lesions after 2-3 weeks from viral injection (medial panel, representative picture), whereas 6 out of 9 Mecp2^{-/y} mice injected with the same dosage (n=9) and treated with cyclosporine (CsA) (10 mg/Kg) were robustly improved (right panel, representative picture). (b) Survival curve of Mecp2^{-/y} mice left GFP-treated (n=10) or injected with a 1*10¹¹ dose of PHP.eB-iMecp2 alone (n=10) or in combination with cyclosporine (CsA). As control, WT littermates were used. (c-e) Spleen cells from Mecp^{2-/y} mice left untreated or injected with a 1*10¹¹ dose of PHP.eB-iMecp2 alone or in combination with CsA or with 1*10¹² dose of PHP.eB-iMecp2 were counted. Frequencies of CD4+ and CD8+ T cell compartments were quantified in the spleen of treated mice by FACS staining. (f) Total splenocytes were labelled with Cell Proliferation Dye eFluor® 670 and stimulated with bone-marrow derived DC transduced with LV-Mecp2 or with anti-CD3 antibodies and proliferation was measured at day 4 by flow cytometry. Mean±SEM of Mecp2^{-/y} mice untreated (n=3) or injected with a 1*10¹¹ dose of PHP.eB-iMecp2 alone (n=3) or in combination with cyclosporine (n=3) or with 1*10¹² dose of PHP.eB-iMecp2 (n=2) are shown. (g) Detection of immune-specific antibody in sera of Mecp2^{-/y} animals mock-treated (KO-GFP) or iMecp2-treated (KO-iMecp2) as well as iMecp2-treated WT animals (WT) was revealed by immunofluorescence assay and compared with a commercial antibody as positive control (Ab). We choose as substrate P19 cells knock-out for the Mecp2 gene and co-transfected with GFP and Mecp2 expression constructs in order to track with GFP the MeCP2+ cells. (h) Similar sera were also tested in Western blot analysis using protein extracts form WT and KO tissue respectively as positive and negative controls. WT P19 extract were also used as positive control (g). Each dot represents one mouse. Error bars, SEM Scale bar: 10 μm. Mann-Whitney U test (two-tailed), with unpaired t-test (c-f), * p<0.05, ** p<0.01, compared groups are indicated by black lines.

FIG. 5

Global gene expression profile of Mecp2^{-/y} cortices transduced with PHP.eB-iMecp2. (a) Venn diagram showing the genes being differentially expressed in the two comparisons, namely Mecp2 KO vs WT, and Mecp2 KO-GT (after gene therapy) vs WT. (b) MA plot showing gene expression fold changes as a function of the average gene expression in the Mecp2 KO-GT vs WT comparison. (c) Representative gene ontology categories highlighting the enrichment for immune response-related datasets being overrepresented in the Mecp2 KO-GT vs WT comparison. (d) MA plot showing gene expression fold changes as a function of the average gene expression in the Mecp2 KO vs WT comparison. (e) MA plot showing gene expression fold changes as a function of the average gene expression in the Mecp2 KO-GT vs KO comparison. (f) Representative gene ontology categories highlighting the enrichment for lipid metabolism-related datasets being overrepresented in the Mecp2 KO vs WT comparison, but not in the Mecp2 KO-GT vs KO comparison. Heatmap showing relative expression of genes belonging to the lipid metabolism-related pathways (g) or Potassium ion transmembrane transport group (h). (i) RT-qPCRs of selected transcript of interest such as Sqle Nsdhl, Msmo1, Kcnc3 and Angpl4 being downregulated in Mecp2 KO and rescued after gene therapy. Red dots in b, d, and e depict differentially expressed genes with FDR 0.1. (j) Representative Western blot and quantitative analysis for the ribosomal protein S6, its phosphorylated form (pS6) and a normaliser (CNX) from cortical tissues of wild-mice and Mecp2 KO transduced with GFP or iMecp2 vector. Error bars, SD. * p<0.05, ** p<0.01 as compared to WT mice (ANOVA-one way with Tukey's post hoc test).

FIG. 6

PHP.eB-mediated iMecp2 gene transfer in wild-type mice is unharmful. (a) High magnification immunostaining for MeCP2 and V5 in cortex, striatum and cerebellum derived from WT brains treated with PHP.eB-iMecp2 (1*10¹¹ and 1*10¹² vg/mouse). Nuclei were stained with DAPI (merge panels). (b) Bar graphs showing the fraction of V5 positive on the total DAPI positive in cortex and striatum (n=6 for 1*10¹¹ and n=3 for 1*10¹² vg/mouse). (c) Western blots analysis to quantify MeCP2 over Actin protein levels in cortex (left panel) and striatum (right panel) derived from WT e, untreated and treated with iMecp2 (1*10¹¹ and 1*10¹² vg/mouse) animals and corresponding densitometric quantification expressed in arbitrary units (right panel) (n=6 for WT, n=6 for 10¹¹ and n=3 for 10¹² vg/mouse) *** p<0.001 as compare to wild-type (WT) untransduced mice (ANOVA-one way, Tukey's post hoc test). Twice a week, mice were tested for (d) body weight, (e) beam balance test and quantified as crossing time (left) and number of errors (right) and (f) general phenotypic assessment evaluated through aggregate severity score (WT untreated [n=8] and treated with iMecp2 virus 1*10¹¹ [n=6], 1*10¹² [n=6], vg/mouse). (g) MA plot showing gene expression fold changes as a function of the average gene expression in the Mecp2 WT-GT (after gene therapy) vs WT comparison (h) Red dots depict differentially expressed genes with FDR 0.1. Heatmap showing relative expression of 1000 random genes in WT and WT-GT, highlighting the lack of differentially expressed genes in the two groups. Error bars, SD. Scale bar: 50 μm.

FIG. 7

Distribution and quantification of iMecp2 gene transfer in Mecp2^{-/y} brains (a) Low magnification of MeCP2 immunostaining of anterior (upper panel) and posterior (cerebellum, lower panel) brains sections in Mecp2^{-/y} control untreated (UT) and treated animals (1*10¹⁰, 1*10¹¹, 1*10¹² vg/mouse). (b) High magnification confocal images for MeCP2 and V5 in cortex derived from WT and iMecp2 treated Mecp2^{-/y} (untreated [WT], 1*10¹¹ and 1*10¹² vg/mouse) animals. Nuclei were stained with DAPI. Scale bar: 10 μm. Left panel: graphs showing the quantification of cellular levels of total MeCP2 detected with anti-MeCP2 immunofluorescence in cells of the cortex and quantified in arbitrary units based on field pixel intensity (n=30 nuclei for per condition, UN: untransduced). Error bars, SD. *** p<0.001 as compared to untreated (WT) (ANOVA-one way, Tukey's post hoc test). Scale bars: 500 μm (a), 10 μm (b).

FIG. 8

Cell type analysis of iMecp2 gene transfer in Mecp2^{-/y} brains (a) Characterisation of iMecp2-transduced cells in Mecp2^{-/y} cortex (1*10¹¹ vg/mouse) using colocalisation of V5+ cells with markers of brain cells sub-populations such as: neurons (marked with NeuN, upper panel), astrocytes (marked with GFAP, middle panel), and GABAergic interneurons (marked with GABA, lower panel). Nuclei were stained with DAPI. All images were captured using confocal microscope. Scale bar: 50 μm. (b) Bar graphs showing co-localisation of these markers with V5 or MeCP2 (n=3) (c) High magnification confocal images of wild-type (WT, upper panel) and iMecp2 transduced nuclei (lower panel) stained with MeCP2 antibody both exhibit heterochromatin-enriched localisation. Error bars, SD. Scale bars: 50 μm (a), 5 μm (c).

FIG. 9

Cell type analysis of iMecp2 gene transfer in Mecp2^{-/y} brains (a) Characterisation of iMecp2-transduced cells in Mecp2^{-/y} cortex (1*10¹¹ vg/mouse) using colocalisation of V5+ cells with markers of brain cells sub-populations such as: neurons (marked with NeuN, upper panel), astrocytes (marked with GFAP, middle panel), and GABAergic interneurons (marked with GABA, lower panel). Nuclei were stained with DAPI. All images were captured using confocal microscope. Scale bar: 50 μm. (b) Bar graphs showing co-localisation of these markers with V5 or MeCP2 (n=3) (c) High magnification confocal images of wild-type (WT, upper panel) and iMecp2 transduced nuclei (lower panel) stained with MeCP2 antibody both exhibit heterochromatin-enriched localisation. Error bars, SD. Scale bars: 50 μm (a), 5 μm (c).

FIG. 10

PHP.eB-iMecp2 gene transfer in Mecp2^{+/-} females. (a) Illustration of the experimental setting to restore the expression of MeCP2 in Mecp2^{+/-} animals by means of AAV-PHP.eB. (b) Mouse body weight was monitored every two weeks and represented as mean of each group. (c) General phenotypic assessment evaluated through aggregate severity score. (d) Spontaneous locomotor activity was tested in a spontaneous field arena and shown as quantification of travelled total distance. All groups of animals were tested for motor coordination using beam balance test and quantified as crossing time (e) and number of errors (f) (WT untreated [n=8], Mecp2^{+/-} untreated [n=6], Mecp2^{+/-} treated with iMecp2 virus 1*10¹¹ vg/mouse [n=6]). Error bars, SD. ** p<0.01. ANOVA-two way (b, c, e, f) or ANOVA-one way (d) with Tukey's post hoc test.

FIG. 11

Characterisation of iMecp2 transgene expression in brain and liver of wild-type animals. Vector biodistribution (upper panel) and transgene expression (lower panel) in mice cortex and liver of WT mice untreated (UT, n=3) and treated with 1*10¹¹ (n=3) or 1*10¹² (n=3) iMecp2. Data were normalised respectively over Mecp2 gene level and expression of WT mice (UT, light blue bar). Error bars, SD.

FIG. 12

Protein levels in PHP.eB-iMecp2 transduced wild-type animals (a) Western blots analysis to quantify MeCP2 over Actin protein levels in striatum (upper panel) and liver (lower panel) derived from WT untreated (UT) and treated with iMecp2 (1*10¹¹ and 1*10¹² vg/mouse) mice. Corresponding densitometric quantification expressed in arbitrary units (n=3 for UT, n=3 for 1*10¹¹; n=3 for 1*10¹² vg/mouse) are shown on the right. (b) High magnification confocal images for V5 and MeCP2 in cortex derived from wild-type untreated (WT) and treated with iMecp2 (1*10¹¹, 1*10¹² vg/mouse) animals. Nuclei were stained with DAPI. Scale bar: 10 μm (Right panel). Left panel: graphs showing the quantification of cellular levels of total MeCP2 detected by immunofluorescence in cells of the cortex and quantified in arbitrary units based on field pixel intensity (n=30 nuclei for per condition, UT: untreated). Error bars, SD. ** p<0.05 and *** p<0.001 compared to UT (ANOVA-one way, Tukey's post hoc test). Scale bar: 10 μm.

FIG. 13

shRNA targeting Mecp2 gene. (a) Schematic showing targeting of the Mecp2 gene 3′-UTR by the shRNA. (b,c) Downregulation of Mecp2 RNA (b) by 50-fold and reduction of MeCP2 protein levels (c) by 70% in mouse primary neurons after 7 days from the transduction with an shRNA-expressing lentivirus.

FIG. 14

Vector comprising the shRNA targeting Mecp2 gene. (a) Schematic showing the AAV vector (shMecp2-iMecp2) comprising the shRNA-encoding sequence. (b) Study of Mecp2 protein levels by Western blotting in wild-type mouse primary cortical neurons transduced with AAV-PHP.eB viral particles comprising the shMecp2-iMecp2 construct.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Rett Syndrome

Rett syndrome (RTT) is a severe neurological disorder and a leading cause of intellectual disabilities in girls. RTT is characterised by a period of 6-12 months of overtly normal development followed by rapid regression with the loss of the purposeful motor skills and the onset of repetitive and autistic behaviours.

In the vast majority of cases RTT is caused by loss-of-function mutations in the MECP2 gene, which encodes the methyl-CpG binding protein 2 (MeCP2) (Bienvenu, T. et al. (2006) Nat Rev Genet 7: 415-426).

Recent studies have revealed that the Mecp2 loss significantly alters neuronal activity leading to a progressive imbalance of the excitatory-inhibitory synaptic activity across the brain with divergent modalities occurring between different circuits and regions of the brain (Banerjee, A. et al. (2016) PNAS 113: E7287-E7296).

Methyl-CpG Binding-Protein 2 (MeCP2)

Methyl-CpG binding protein 2 (MeCP2) is a global chromatin regulator highly expressed in neurons.

In some embodiments, the MeCP2 is human or mouse MeCP2. In preferred embodiments, the MeCP2 is human MeCP2.

An example MeCP2 sequence is:

(SEQ ID NO: 1; human)
MAAAAAAAPSGGGGGGEEERLEEKSEDQDLQGLKDKPLKFKKVKKDKKEE
KEGKHEPVQPSAHHSAEPAEAGKAETSEGSGSAPAVPEASASPKQRRSII
RDRGPMYDDPTLPEGWTRKLKQRKSGRSAGKYDVYLINPQGKAFRSKVEL
IAYFEKVGDTSLDPNDFDFTVTGRGSPSRREQKPPKKPKSPKAPGTGRGR
GRPKGSGTTRPKAATSEGVQVKRVLEKSPGKLLVKMPFQTSPGGKAEGGG
ATTSTQVMVIKRPGRKRKAEADPQAIPKKRGRKPGSVVAAAAAEAKKKAV
KESSIRSVQETVLPIKKRKTRETVSIEVKEVVKPLLVSTLGEKSGKGLKT
CKSPGRKSKESSPKGRSSSASSPPKKEHHHHHHHSESPKAPVPLLPPLPP
PPPEPESSEDPTSPPEPQDLSSSVCKEEKMPRGGSLESDGCPKEPAKTQP
AVATAATAAEKYKHRGEGERKDIVSSSMPRPNREEPVDSRTPVTERVS

A further example MeCP2 sequence is:

(SEQ ID NO: 2; mouse)
MAAAAATAAAAAAPSGGGGGGEEERLEEKSEDQDLQGLRDKPLKFKKAKK
DKKEDKEGKHEPLQPSAHHSAEPAEAGKAETSESSGSAPAVPEASASPKQ
RRSIIRDRGPMYDDPTLPEGWTRKLKQRKSGRSAGKYDVYLINPQGKAFR
SKVELIAYFEKVGDTSLDPNDFDFTVTGRGSPSRREQKPPKKPKSPKAPG
TGRGRGRPKGSGTGRPKAAASEGVQVKRVLEKSPGKLVVKMPFQASPGGK
GEGGGATTSAQVMVIKRPGRKRKAEADPQAIPKKRGRKPGSVVAAAAAEA
KKKAVKESSIRSVHETVLPIKKRKTRETVSIEVKEVVKPLLVSTLGEKSG
KGLKTCKSPGRKSKESSPKGRSSSASSPPKKEHHHHHHHSESTKAPMPLL
PSPPPPEPESSEDPISPPEPQDLSSSICKEEKMPRGGSLESDGCPKEPAK
TQPMVATTTTVAEKYKHRGEGERKDIVSSSMPRPNREEPVDSRTPVTERV
S

An example nucleotide sequence encoding MeCP2 is:

(SEQ ID NO: 3; human)
ATGGCCGCCGCCGCCGCCGCCGCGCCGAGCGGAGGAGGAGGAGGAGGCGA
GGAGGAGAGACTGGAAGAAAAGTCAGAAGACCAGGACCTCCAGGGCCTCA
AGGACAAACCCCTCAAGTTTAAAAAGGTGAAGAAAGATAAGAAAGAAGAG
AAAGAGGGCAAGCATGAGCCCGTGCAGCCATCAGCCCACCACTCTGCTGA
GCCCGCAGAGGCAGGCAAAGCAGAGACATCAGAAGGGTCAGGCTCCGCCC
CGGCTGTGCCGGAAGCTTCTGCCTCCCCCAAACAGCGGCGCTCCATCATC
CGTGACCGGGGACCCATGTATGATGACCCCACCCTGCCTGAAGGCTGGAC
ACGGAAGCTTAAGCAAAGGAAATCTGGCCGCTCTGCTGGGAAGTATGATG
TGTATTTGATCAATCCCCAGGGAAAAGCCTTTCGCTCTAAAGTGGAGTTG
ATTGCGTACTTCGAAAAGGTAGGCGACACATCCCTGGACCCTAATGATTT
TGACTTCACGGTAACTGGGAGAGGGAGCCCCTCCCGGCGAGAGCAGAAAC
CACCTAAGAAGCCCAAATCTCCCAAAGCTCCAGGAACTGGCAGAGGCCGG
GGACGCCCCAAAGGGAGCGGCACCACGAGACCCAAGGCGGCCACGTCAGA
GGGTGTGCAGGTGAAAAGGGTCCTGGAGAAAAGTCCTGGGAAGCTCCTTG
TCAAGATGCCTTTTCAAACTTCGCCAGGGGGCAAGGCTGAGGGGGGTGGG
GCCACCACATCCACCCAGGTCATGGTGATCAAACGCCCCGGCAGGAAGCG
AAAAGCTGAGGCCGACCCTCAGGCCATTCCCAAGAAACGGGGCCGAAAGC
CGGGGAGTGTGGTGGCAGCCGCTGCCGCCGAGGCCAAAAAGAAAGCCGTG
AAGGAGTCTTCTATCCGATCTGTGCAGGAGACCGTACTCCCCATCAAGAA
GCGCAAGACCCGGGAGACGGTCAGCATCGAGGTCAAGGAAGTGGTGAAGC
CCCTGCTGGTGTCCACCCTCGGTGAGAAGAGCGGGAAAGGACTGAAGACC
TGTAAGAGCCCTGGGCGGAAAAGCAAGGAGAGCAGCCCCAAGGGGCGCAG
CAGCAGCGCCTCCTCACCCCCCAAGAAGGAGCACCACCACCATCACCACC
ACTCAGAGTCCCCAAAGGCCCCCGTGCCACTGCTCCCACCCCTGCCCCCA
CCTCCACCTGAGCCCGAGAGCTCCGAGGACCCCACCAGCCCCCCTGAGCC
CCAGGACTTGAGCAGCAGCGTCTGCAAAGAGGAGAAGATGCCCAGAGGAG
GCTCACTGGAGAGCGACGGCTGCCCCAAGGAGCCAGCTAAGACTCAGCCC
GCGGTTGCCACCGCCGCCACGGCCGCAGAAAAGTACAAACACCGAGGGGA
GGGAGAGCGCAAAGACATTGTTTCATCCTCCATGCCAAGGCCAAACAGAG
AGGAGCCTGTGGACAGCCGGACGCCCGTGACCGAGAGAGTTAGCTGA

A further example nucleotide sequence encoding MeCP2 is:

(SEQ ID NO: 4; mouse)
ATGGCCGCCGCTGCCGCCACCGCCGCCGCCGCCGCCGCGCCGAGCGGAGG
AGGAGGAGGAGGCGAGGAGGAGAGACTGGAGGAAAAGTCAGAAGACCAGG
ATCTCCAGGGCCTCAGAGACAAGCCACTGAAGTTTAAGAAGGCGAAGAAA
GACAAGAAGGAGGACAAAGAAGGCAAGCATGAGCCACTACAACCTTCAGC
CCACCATTCTGCAGAGCCAGCAGAGGCAGGCAAAGCAGAAACATCAGAAA
GCTCAGGCTCTGCCCCAGCAGTGCCAGAAGCCTCGGCTTCCCCCAAACAG
CGGCGCTCCATTATCCGTGACCGGGGACCTATGTATGATGACCCCACCTT
GCCTGAAGGTTGGACACGAAAGCTTAAACAAAGGAAGTCTGGCCGATCTG
CTGGAAAGTATGATGTATATTTGATCAATCCCCAGGGAAAAGCTTTTCGC
TCTAAAGTAGAATTGATTGCATACTTTGAAAAGGTGGGAGACACCTCCTT
GGACCCTAATGATTTTGACTTCACGGTAACTGGGAGAGGGAGCCCCTCCA
GGAGAGAGCAGAAACCACCTAAGAAGCCCAAATCTCCCAAAGCTCCAGGA
ACTGGCAGGGGTCGGGGACGCCCCAAAGGGAGCGGCACTGGGAGACCAAA
GGCAGCAGCATCAGAAGGTGTTCAGGTGAAAAGGGTCCTGGAGAAGAGCC
CTGGGAAACTTGTTGTCAAGATGCCTTTCCAAGCATCGCCTGGGGGTAAG
GGTGAGGGAGGTGGGGCTACCACATCTGCCCAGGTCATGGTGATCAAACG
CCCTGGCAGAAAGCGAAAAGCTGAAGCTGACCCCCAGGCCATTCCTAAGA
AACGGGGTAGAAAGCCTGGGAGTGTGGTGGCAGCTGCTGCAGCTGAGGCC
AAAAAGAAAGCCGTGAAGGAGTCTTCCATACGGTCTGTGCATGAGACTGT
GCTCCCCATCAAGAAGCGCAAGACCCGGGAGACGGTCAGCATCGAGGTCA
AGGAAGTGGTGAAGCCCCTGCTGGTGTCCACCCTTGGTGAGAAAAGCGGG
AAGGGACTGAAGACCTGCAAGAGCCCTGGGCGTAAAAGCAAGGAGAGCAG
CCCCAAGGGGCGCAGCAGCAGTGCCTCCTCCCCACCTAAGAAGGAGCACC
ATCATCACCACCATCACTCAGAGTCCACAAAGGCCCCCATGCCACTGCTC
CCATCCCCACCCCCACCTGAGCCTGAGAGCTCTGAGGACCCCATCAGCCC
CCCTGAGCCTCAGGACTTGAGCAGCAGCATCTGCAAAGAAGAGAAGATGC
CCCGAGGAGGCTCACTGGAAAGCGATGGCTGCCCCAAGGAGCCAGCTAAG
ACTCAGCCTATGGTCGCCACCACTACCACAGTTGCAGAAAAGTACAAACA
CCGAGGGGAGGGAGAGCGCAAAGACATTGTTTCATCTTCCATGCCAAGGC
CAAACAGAGAGGAGCCTGTGGACAGCCGGACGCCCGTGACCGAGAGAGTT
AGCTGA

In some embodiments, the MeCP2 is encoded by a nucleotide sequence that has at least 70%, 80%, 90%, 95%, 96%, 97%, 98% 99% or 100% identity to SEQ ID NO: 3 or 4 (preferably SEQ ID NO: 3), preferably wherein the protein encoded by the nucleotide sequence substantially retains the natural function of the protein represented by any one of SEQ ID NOs: 1 or 2.

In some embodiments, the MeCP2 is encoded by a nucleotide sequence that encodes an amino acid sequence that has at least 70%, 80%, 90%, 95%, 96%, 97%, 98% 99% or 100% identity to SEQ ID NO: 1 or 2 (preferably SEQ ID NO: 1), preferably wherein the amino acid sequence substantially retains the natural function of the protein represented by SEQ ID NOs: 1 or 2.

In some embodiments, the MeCP2 comprises or consists of an amino acid sequence that has at least 70%, 80%, 90%, 95%, 96%, 97%, 98% 99% or 100% identity to SEQ ID NO: 1 or 2 (preferably SEQ ID NO: 1), preferably wherein the amino acid sequence substantially retains the natural function of the protein represented by SEQ ID NOs: 1 or 2.

Blood-Brain Barrier (BBB)

The term “blood brain barrier” (BBB) as used herein means the highly selective semi-permeable membrane barrier which separates the circulating blood from the brain and extracellular fluid in the central nervous system. It is formed by the selectivity of tight junctions between endothelial cells. The blood-brain barrier occurs along all capillaries of the brain and consists of tight junctions.

Overcoming the difficulty of delivering therapeutic agents to specific regions of the brain presents a major challenge to treatment of most brain disorders.

Preferably, the vector particle (e.g. the AAV vector particle) is adapted for crossing an intact blood brain barrier. Preferably the vector particle (e.g. the AAV vector particle) does not impair blood-brain barrier integrity and/or selectivity and/or affect permeability.

In other words, the vector particle is adapted to cross a blood-brain barrier which has not been compromised or weakened, i.e. which maintains tight junctions between endothelial cells. Methods are known in the art which can determine whether or not a blood brain barrier is intact. For example, the permeability of the blood-brain barrier can be detected by perfusion of Evan's blue dye. Alternatively a fluorescent-conjugated cadaverine dye can be used as a blood-brain barrier permeability marker, together with the AAV particle carrying a fluorescent marker.

Preferably, the vector particle (e.g. the AAV vector particle) does not cause microgliosis. Suitably, the vector particle (e.g. the AAV vector particle) does not cause sustained inflammation in the central nervous system.

The “central nervous system” as used herein means the nervous system consisting of the brain and spinal cord.

The “peripheral nervous system” as used herein means the components of the nervous system outside of the central nervous system. The peripheral nervous system consists of the nerves and ganglia outside of the brain and spinal cord.

Promoters and Regulatory Sequences

The polynucleotides and vectors of the invention include elements allowing for the expression of MeCP2 in vitro or in vivo. These may be referred to as expression control sequences. Thus, the polynucleotides and vectors typically comprise expression control sequences (e.g. comprising a promoter sequence) operably linked to the nucleotide sequence encoding the transgene.

Any suitable strong promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter such as an endothelial specific promoter). The promoter may show inducible expression in response to presence of another factor, for example a factor present in a host cell.

In any event, where the polynucleotide or vector is administered for therapy, it is preferred that the promoter should be functional in the target cell background.

In some embodiments, the promoter is neural-cell specific. In some embodiments, the promoter is astrocyte specific.

In some embodiments, the promoter is selected from the group consisting of a chicken β-actin (CBA) promoter, a β-actin promoter, a CAG promoter, a cytomegalovirus (CMV) promoter, a human elongation factor-1-alpha (HEF-1-alpha), a Chinese hamster elongation factor-1-alpha (CHEF-1-alpha) promoter and a phosphoglycerate kinase (PGK) promoter.

In preferred embodiments, the promoter is a chicken β-actin (CBA) promoter.

An example CBA promoter is:

(SEQ ID NO: 5)
TCGAGGTGAGCCCCACGTTCTGCTTCACTCTCCCCATCTCCCCCCCCTCC
CCACCCCCAATTTTGTATTTATTTATTTTTTAATTATTTTGTGCAGCGAT
GGGGGCGGGGGGGGGGGGGGGGCGCGCGCCAGGCGGGGCGGGGCGGGGCG
AGGGGCGGGGCGGGGCGAGGCGGAGAGGTGCGGCGGCAGCCAATCAGAGC
GGCGCGCTCCGAAAGTTTCCTTTTATGGCGAGGCGGCGGCGGCGGCGGCC
CTATAAAAAGCGAAGCGCGCGGCGGGCG

In some embodiments, the CBA promoter comprises or consists of a nucleotide sequence that has at least 70%, 80%, 90%, 95%, 96%, 97%, 98% 99% or 100% identity to SEQ ID NO: 5.

The chicken beta-actin (CBA) promoter may optionally be used in combination with a cytomegalovirus (CMV) enhancer element.

The polynucleotide or vector of the invention comprise a 3′-UTR that is less than or equal to about 1000 bp in length.

In preferred embodiments, the 3′-UTR is derived from the MeCP2 3′-UTR (e.g. is a truncated form thereof). In preferred embodiments, the 3′-UTR is a truncated MeCP2 3′UTR.

Preferably, the 3′-UTR (e.g. the MeCP2 3′-UTR) is truncated at the 3′ end (e.g. retains its natural 5′ end).

In some embodiments, the 3′-UTR is a synthetic UTR assembled from regulatory elements in the wild type (8.6 kb long) 3′-UTR, as described in Matagne, V. et al. (2017) Neurobiol. Dis. 99: 1-11.

An example 3′-UTR sequence is:

(SEQ ID NO: 6; mouse)
CTTTACATAGAGCGGATTGCAAAGCAAACCAACAAGAATAAAGGCAGCTG
TTGTCTCTTCTCCTTATGGGTAGGGCTCTGACAAAGCTTCCCGATTAACT
GAAATAAAAAATATTTTTTTTTCTTTCAGTAAACTTAGAGTTTCGTGGCT
TCGGGGTGGGAGTAGTTGGAGCATTGGGATGTTTTTCTTACCGACAAGCA
CAGTCAGGTTGAAGACCTAACCA

A further example 3′-UTR sequence is:

(SEQ ID NO: 7; human)
CTTTACACGGAGCGGATTGCAAAGCAAACCAACAAGAATAAAGGCAGCT
GTTGTCTCTTCTCCTTATGGGTAGGGCTCTGACAAAGCTTCCCGATTAA
CTGAAATAAAAAATATTTTTTTTTCTTTCAGTAAACTTAGAGTTTCGTG
GCTTCAGGGTGGGAGTAGTTGGAGCATTGGGGATGTTTTTCTTACCGAC
AAGCACAGTCAGGTTGAAGACCTAACCA

A further example 3′-UTR sequence is:

(SEQ ID NO: 8; human)
CTTTACACGGAGCGGATTGCAAAGCAAACCAACAAGAATAAAGGCAGCT
GTTGTCTCTTCTCCTTATGGGTAGGGCTCTGACAAAGCTTCCCGATTAA
CTGAAATAAAAAATATTTTTTTTTCTTTCAGTAAAAAAAAAAAAAAAAA
AAA

In some embodiments, the 3′-UTR comprises or consists of a nucleotide sequence that has at least 70%, 80%, 90%, 95%, 96%, 97%, 98% 99% or 100% identity to any one of SEQ ID NOs: 6-8.

The polynucleotide or vector of the invention may also comprise one or more additional regulatory sequences which may act pre- or post-transcriptionally.

Regulatory sequences are any sequences which facilitate expression of the transgene, i.e. act to increase expression of a transcript, improve nuclear export of mRNA or enhance its stability. Such regulatory sequences include for example enhancer elements, post-transcriptional regulatory elements and polyadenylation sites. An example of a polyadenylation site is the Human or Bovine Growth Hormone poly-A signal.

An example human growth hormone poly-A sequence is:

(SEQ ID NO: 9)
TCGAGAGATCTACGGGTGGCATCCCTGTGACCCCTCCCCAGTGCCTCTC
CTGGCCCTGGAAGTTGCCACTCCAGTGCCCACCAGCCTTGTCCTAATAA
AATTAAGTTGCATCATTTTGTCTGACTAGGTGTCCTTCTATAATATTAT
GGGGTGGAGGGGGGTGGTATGGAGCAAGGGGCAAGTTGGGAAGACAACC
TGTAGGGCCTGCGGGGTCTATTGGGAACCAAGCTGGAGTGCAGTGGCAC
AATCTTGGCTCACTGCAATCTCCGCCTCCTGGGTTCAAGCGATTCTCCT
GCCTCAGCCTCCCGAGTTGTTGGGATTCCAGGCATGCATGACCAGGCTC
AGCTAATTTTTGTTTTTTTGGTAGAGACGGGGTTTCACCATATTGGCCA
GGCTGGTCTCCAACTCCTAATCTCAGGTGATCTACCCACCTTGGCCTCC
CAAATTGCTGGGATTACAGGCGTGAACCACTGCTCCCTTCCCTGTCCTT

In some embodiments, the human growth hormone poly-A sequence comprises or consists of a nucleotide sequence that has at least 70%, 80%, 90%, 95%, 96%, 97%, 98% 99% or 100% identity to SEQ ID NO: 9.

An example of a post-transcriptional regulatory element for use in a polynucleotide or vector of the invention is the woodchuck hepatitis post-transcriptional regulatory element (WPRE) or a variant thereof.

Another regulatory sequence which may be used in a polynucleotide or vector of the invention is a scaffold-attachment region (SAR). Additional regulatory sequences may be readily selected by the skilled person.

Inhibitor of MeCP2 Expression

In some embodiments, the polynucleotide further comprises a nucleotide sequence encoding an inhibitor of MeCP2 expression.

The term “inhibitor”, as used herein in the context of inhibition of MeCP2 expression, may refer to an agent that reduces the expression of MeCP2 relative to the level of MeCP2 expression in the absence of the agent, but under otherwise substantially identical conditions. The inhibitor may, for example, reduce expression of MeCP2 (preferably endogenous MeCP2) by at least 10%, 20%, 30%, 40%, 50%, 60%, 70%, 80%, 90% or 95% relative to the level of MeCP2 expression in its absence. Preferably, the inhibitor reduces expression of MeCP2 by at least 70% relative to the level of MeCP2 expression in its absence. In some embodiments, the inhibitor prevents expression of MeCP2 (preferably endogenous MeCP2) entirely.

Expression levels of a protein, such as MeCP2, may be readily measured and quantified by the skilled person using techniques well known in the art, for example using Western blotting.

In some embodiments, the inhibitor is specific for endogenous MeCP2.

In some embodiments, the polynucleotide further comprises a nucleotide sequence encoding an inhibitor of endogenous MeCP2 expression (e.g. inhibits expression of MeCP2 that is endogenous to a cell into which the polynucleotide is introduced).

In some embodiments, the inhibitor does not inhibit expression of the MeCP2 encoded by the polynucleotide of the invention. In some embodiments, the inhibitor substantially does not inhibit expression of the MeCP2 encoded by the polynucleotide of the invention.

In some embodiments, the inhibitor inhibits expression of endogenous MeCP2 more than it inhibits expression of the MeCP2 encoded by the polynucleotide of the invention. For example, the inhibitor may inhibit expression of endogenous MeCP2 by at least 1.5-fold, 2-fold, 2.5-fold, 5-fold, 10-fold, 20-fold, 50-fold, 100-fold or 1000-fold more than it inhibits expression of the MeCP2 encoded by the polynucleotide of the invention.

In some embodiments, the inhibitor targets the 3′-UTR of a gene (preferably an endogenous gene) encoding MeCP2. In some embodiments, the section of the 3′-UTR targeted by the inhibitor is not comprised in the polynucleotide of the invention.

In some embodiments, the inhibitor is an shRNA, siRNA, miRNA or antisense DNA/RNA. Preferably, the inhibitor is an shRNA.

An example nucleotide sequence encoding an shRNA that inhibits expression of MeCP2 is:

(shMecp2)
(SEQ ID NO: 22)
gattgtagattcaggttaa

In some embodiments, the polynucleotide comprises or consists of a nucleotide sequence that has at least 70%, 80%, 90%, 95%, 96%, 97%, 98% 99% or 100% identity to SEQ ID NO: 24.

siRNAs, shRNAs, miRNAs and Antisense DNAs/RNAs

Inhibition (e.g. of the MeCP2) may be achieved using post-transcriptional gene silencing (PTGS). Post-transcriptional gene silencing mediated by double-stranded RNA (dsRNA) is a conserved cellular defense mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) noncoding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein are well known in the art.

Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid and/or facilitating the expression of the protein encoded by a segment of nucleic acid.

Vectors comprising polynucleotides used in the invention may be introduced into cells using a variety of techniques known in the art, such as transfection, transduction and transformation.

Transfection may refer to a general process of incorporating a nucleic acid into a cell and includes a process using a non-viral vector to deliver a polynucleotide to a cell. Transduction may refer to a process of incorporating a nucleic acid into a cell using a viral vector.

Preferably, the vectors used to transduce cells in the invention are viral vectors. The vectors of the invention are preferably adeno-associated viral (AAV) vectors, although it is contemplated that other viral vectors may be used.

Preferably, the viral vector for use according to the present invention is in the form of a viral vector particle.

In one aspect the invention provides a viral vector particle adapted for crossing the blood-brain barrier, wherein the viral vector particle comprises a nucleotide sequence encoding methyl-CpG binding-protein 2 (MeCP2) operably linked to a strong promoter and a 3′-UTR, wherein the 3′-UTR is less than or equal to about 1000 bp in length.

In one embodiment the viral vector particle adapted for crossing the blood-brain barrier for use according to the invention is a retroviral, lentiviral, adeno-associated viral (AAV) or adenoviral vector particle. Preferably, the viral vector particle is a lentiviral or AAV vector particle, more preferably an AAV vector particle.

Although, some embodiments of the invention have been described with respect to AAV vector particles, it will be appreciated that some embodiments may apply mutatis mutandis to other viral vectors disclosed herein.

Adeno-Associated Viral (AAV) Vectors

In one aspect the invention provides a AAV vector particle, wherein the AAV vector particle comprises a nucleotide sequence encoding methyl-CpG binding-protein 2 (MeCP2) operably linked to a strong promoter and a 3′-UTR, wherein the 3′-UTR is less than or equal to about 1000 bp in length. In some embodiments, the AAV vector particle is adapted for crossing the blood-brain barrier.

Methods of preparing and modifying viral vectors and viral vector particles, such as those derived from AAV, are well known in the art.

The AAV vector may comprise an AAV genome or a fragment or derivative thereof.

An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain.

Several rAAV vectors have been reported to efficiently cross the blood-brain barrier and transduce neurons and astrocytes in the neonatal mouse central nervous system (Zhang et al., Molecular therapy vol. 19, no 8, 1440-1448).

In some embodiments, the AAV is an AAV1, AAV6, AAV6.2, AAV7, AAV9, rh10, rh39 or rh43 serotype. In some embodiments, the AAV vector particle comprises an AAV1, AAV6, AAV6.2, AAV7, AAV9, rh10, rh39 or rh43 serotype capsid protein. In some embodiments, the AAV vector particle is an AAV1, AAV6, AAV6.2, AAV7, AAV9, rh10, rh39 or rh43 vector particle.

In some embodiments, the AAV is an AAV9; AAV9 PHP.B; AAV9 PHP.eB; or AAVrh10 serotype. In some embodiments, the AAV vector particle comprises an AAV9; AAV9 PHP.B; AAV9 PHP.eB; or AAVrh10 serotype capsid protein.

The capsid protein may be an artificial or mutant capsid protein.

The term “artificial capsid” as used herein means that the capsid particle comprises an amino acid sequence which does not occur in nature or which comprises an amino acid sequence which has been engineered (e.g. modified) from a naturally occurring capsid amino acid sequence.

In other words the artificial capsid protein comprises a mutation or a variation in the amino acid sequence compared to the sequence of the parent capsid from which it is derived where the artificial capsid amino acid sequence and the parent capsid amino acid sequences are aligned. Methods of sequence alignment are well known in the art and referenced herein.

The term “adapted for crossing the blood brain barrier” as used herein means that the vector particle has the ability to cross the blood brain barrier, for example the vector particle may comprise a mutation or modification relative to the wild type vector particle which improves the ability to cross the blood brain barrier relative to an unmodified or wild type viral particle. Improved ability to cross the blood brain barrier may be measured for example by measuring the expression of a transgene, e.g. GFP, carried by the vector particle, wherein expression of the transgene in the brain correlates with the ability of the viral particle to cross the blood brain barrier.

In some embodiments, the AAV vector particle comprises an artificial capsid amino acid sequence which enables the viral particle to cross the blood-brain barrier.

In some embodiments, the AAV vector particle capable of crossing the blood-brain barrier comprises a VP1 capsid protein comprising an amino acid sequence comprising at least four contiguous amino acids from the sequence TLAVPFK (SEQ ID NO: 14) or KFPVALT (SEQ ID NO: 15).

In some embodiments, the AAV vector particle capable of crossing the blood-brain barrier comprises a VP1 capsid protein comprising an amino acid sequence comprising at least five contiguous amino acids from the sequence TLAVPFK (SEQ ID NO: 14) or KFPVALT (SEQ ID NO: 15).

In some embodiments, the AAV vector particle capable of crossing the blood-brain barrier comprises a VP1 capsid protein comprising an amino acid sequence comprising at least six contiguous amino acids from the sequence TLAVPFK (SEQ ID NO: 14) or KFPVALT (SEQ ID NO: 15).

In some embodiments, the AAV vector particle capable of crossing the blood-brain barrier comprises a VP1 capsid protein comprising an amino acid sequence comprising the sequence TLAVPFK (SEQ ID NO: 14) or KFPVALT (SEQ ID NO: 15).

In some embodiments, the nucleic acid sequence encoding the at least four, at least five, at least six or all seven contiguous amino acids from the sequence TLAVPFK (SEQ ID NO: 14) or KFPVALT (SEQ ID NO: 15) is inserted at a position corresponding to the position between a sequence encoding for amino acids 588 and 589 of AAV9 (SEQ ID NO: 10).

An example amino acid sequence of the (wild-type) AAV9 capsid is:

(SEQ ID NO: 10)
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
AQAQTGWVQNQGILPGMVWQDRDVYLQGPIWAKIPHTDGNFHPSPLMGG
FGMKHPPPQILIKNTPVPADPPTAFNKDKLNSFITQYSTGQVSVEIEWE
LQKENSKRWNPEIQYTSNYYKSNNVEFAVNTEGVYSEPRPIGTRYLTRN
L

In some embodiments, the AAV vector particle comprises a AAV9 PHP.B capsid, preferably the AAV-PHP.B VP1 capsid protein.

In some embodiments, the AAV vector particle capable of crossing the blood-brain barrier is AAV9 PHP.B.

In some embodiments, the amino acid sequence of the AAV-PHP.B capsid VP1 protein is:

(SEQ ID NO: 11)
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSRTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
TLAVPFKAQAQTGWVQNQGILPGMVWQDRDVYLQGPIWAKIPHIDGNFH
PSPLMGGFGMKHPPPQILIKNTPVPADPPTAFNKDKLNSFITQYSTGQV
SVEIEWELQKENSKRWNPEIQYISNYYKSNNVEFAVNTEGVYSEPRPIG
TRYLTRNL

In some embodiments, the AAV vector particle comprises a capsid comprising an amino acid sequence that has at least 70%, 75%, 80%, 85% or 90% identity to SEQ ID NO: 11, more preferably at least 95%, 96%, 97%, 98%, 99% or 100% identity to SEQ ID NO: 11, wherein the AAV vector particle is capable of crossing the blood-brain barrier.

The AAV-PHP.B vector is described in Deverman et al. (2016) Nat Biotechnol 34: 204-209 and WO 2015/038958, which are incorporated herein by reference.

In some embodiments, the AAV vector particle capable of crossing the blood-brain barrier comprises a VP1 capsid protein comprising an amino acid sequence comprising the sequence DGTLAVPFKAQ (SEQ ID NO: 16).

In some embodiments, the AAV vector particle capable of crossing the blood-brain barrier is AAV9 PHP.eB.

In some embodiments, the amino acid sequence of the AAV-PHP.eB capsid VP1 protein is:

(SEQ ID NO: 12)
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSDG
TLAVPFKAQAQTGWVQNQGILPGMVWQDRDVYLQGPIWAKIPHTDGNFH
PSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIG
TRYLTRNL

In some embodiments, the AAV vector particle comprises a capsid comprising an amino acid sequence that has at least 70%, 75%, 80%, 85% or 90% identity to SEQ ID NO: 12, more preferably at least 95%, 96%, 97%, 98%, 99% or 100% identity to SEQ ID NO: 12, wherein the AAV vector particle is capable of crossing the blood-brain barrier.

The AAV-PHP.eB vector is described in WO 2017/100671, which is incorporated herein by reference.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, clade, clone or isolate of AAV for use in the invention on the basis of their common general knowledge.

The AAV serotype determines the tissue specificity of infection (or tropism) of an AAV virus.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. In preferred embodiments, one or more ITR sequences flank the nucleotide sequences encoding the MeCP2 nucleotide sequence. The AAV genome may also comprise packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle.

A promoter will be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters are generally used to express the rep gene, while the p40 promoter is generally used to express the cap gene.

As discussed above, the AAV genome used in the AAV vector of the invention may therefore be the full genome of a naturally occurring AAV. For example, a vector comprising a full AAV genome may be used to prepare an AAV vector or vector particle in vitro. However, while such a vector may in principle be administered to patients, this will rarely be done in practice. Preferably the AAV genome will be derivatised for the purpose of administration to patients. Such derivatisation is standard in the art and the invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

In some embodiments, the AAV vector comprises at least one, such as two, AAV1, 2, 3, 4, 5, 6, 7, 8, 9, 10, or 11 ITRs. In some embodiments, the AAV vector comprises at least one AAV9 ITR.

In some embodiments, the AAV vector comprises two AAV9 ITRs.

The one or more ITRs will preferably flank the nucleotide sequence encoding MeCP2 at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). Thus, in one embodiment the AAV vector is in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population (e.g. to brain microvascular endothelial cells). The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector of the invention may take the form of a nucleotide sequence comprising an AAV genome or derivative thereof and a sequence encoding the MeCP2 transgene or derivatives thereof.

The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. The AAV particle also includes chemically modified forms bearing ligands adsorbed to the capsid surface. For example, such ligands may include antibodies for targeting a particular cell surface receptor.

Thus, for example, the AAV particles of the invention include those with an AAV2 genome and AAV9 capsid proteins (AAV2/9), or AAV9 PHP.B or PHP.eB capsid proteins.

The AAV vector may comprise multiple copies (e.g., 2, 3 etc.) of the nucleotide sequence referred to herein.

Retroviral and Lentiviral Vectors

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, P et al. (1992) EMBO J. 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above.

Adenoviral Vectors

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

Variants, Derivatives, Analogues, Homologues and Fragments

In addition to the specific proteins and nucleotides mentioned herein, the invention also encompasses variants, derivatives, analogues, homologues and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar—uncharged | C S T M |
| | | N Q |
| | Polar—charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 96% or 97% or 98% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

Method of Treatment

All references herein to treatment include curative, palliative and prophylactic treatment. The treatment of mammals, particularly humans, is preferred. Both human and veterinary treatments are within the scope of the invention.

In some embodiments, the method of treatment provides MeCP2 to the central nervous system of a subject.

In some embodiments, the method of treatment provides MeCP2 to the somatosensory cortex and/or striatum of a subject.

In some embodiments, the method of treatment provides MeCP2 to glial and/or neuronal cells, preferably dopaminergic neurons.

In some embodiments, the method of treatment provides MeCP2 to astrocytes.

In some embodiments, the method of treatment provides MeCP2 to GABAergic neurons. In some embodiments, the method of treatment provides MeCP2 to the cortical GABAergic interneurons.

In some embodiments, the method of treatment provides an improvement in motor function in a subject. Methods for measuring motor function are known to those skilled in the art, for example, the beam balance test disclosed herein.

In some embodiments, the method of treatment provides an improvement in learning and/or cognitive function in a subject. Methods for measuring learning and/or cognitive function are known to those skilled in the art. For example, in humans the General Practitioner Assessment of Cognition (GPCOG) test may be used. Alternative cognitive tests include but are not limited to the Mini Mental State Examination (MMSE), The Six-item Cognitive Impairment Test (6CIT), Abbreviated Mental Test (AMT) and Informant Questionnaire on Cognitive Decline in the Elderly (IQCODE).

Advantageously, the present invention provides a method for treatment by systemically administering the vector particle of the invention.

Pharmaceutical Compositions and Injected Solutions

Although the agents for use in the invention can be administered alone, they will generally be administered in admixture with a pharmaceutical carrier, excipient or diluent, particularly for human therapy.

The medicaments, for example vector particles, of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, e.g. intravenous or intra-arterial.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

Administration

In some embodiments, the polynucleotide, vector or cell is administered to a subject systemically.

In some embodiments, the polynucleotide, vector or cell is administered to a subject locally.

In some embodiments, the polynucleotide, vector or cell is administered to a subject intracranially, intracerebrally or intraparenchymally.

The term “systemic delivery” or “systemic administration” as used herein means that the agent of the invention is administered into the circulatory system, for example to achieve broad distribution of the agent. In contrast, topical or local administration restricts the delivery of the agent to a localised area e.g. intracerebral administration entails direct injection into the brain.

In some embodiments, the polynucleotide, vector or cell is administered intravascularly, intravenously or intra-arterially.

Suitably, in some embodiments the polynucleotide, vector or cell is administered to the internal carotid artery.

As used herein, the term “agent” may refer to the polynucleotide, vector, cell or pharmaceutical composition of the invention.

In some embodiments, the polynucleotide, vector or cell is administered simultaneously, sequentially or separately in combination with an immunosuppressant.

In some embodiments, the immunosuppressant is cyclosporin A (CsA).

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

Dosage

The skilled person can readily determine an appropriate dose of an agent of the invention to administer to a subject. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

In some embodiments, the vector is administered at a dosage of 10⁸ to 10¹² vg/20 g, preferably 10⁹ to 10¹¹ vg/20 g.

In preferred embodiments, the vector is administered at a dosage of 10¹⁰ to 10¹¹ vg/20 g.

Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is human.

In one embodiment the subject is a mouse model of Rett disease.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

Results

Designing an Instable MECP2 Transgene Cassette with Reduced Translation Efficiency

We initially confirmed that the totality of primary mouse neurons in culture can be transduced with the PHP.eB virus.

Then, we generated a transgene cassette with the Mecp2_e1 isoform including the coding sequence and a short 3′-UTR (˜200 bp). This Mecp2 transcript occurs naturally in embryonic stem cells, but during development of the neural system this form is progressively overcome by transcripts with longer 3′-UTRs (e.g. 8.6 kb).

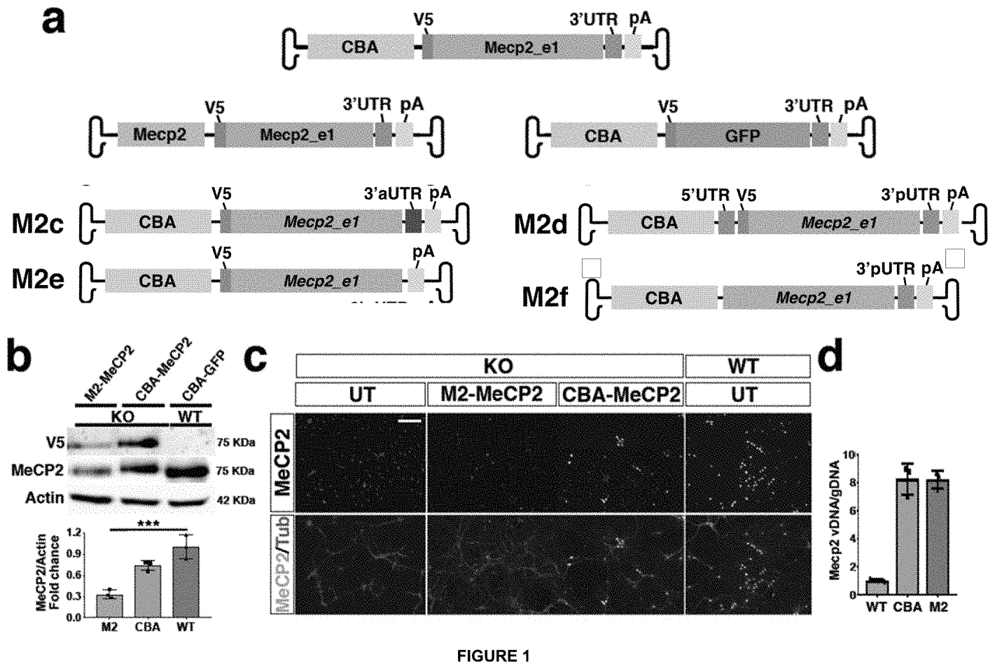

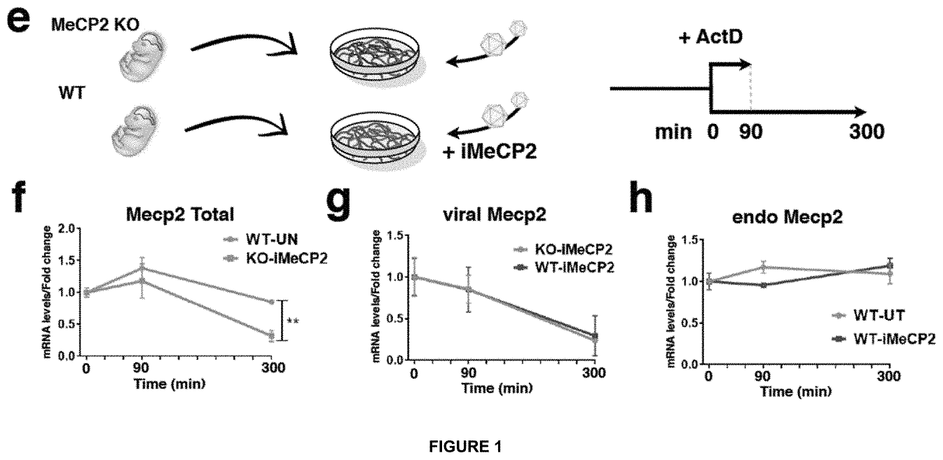

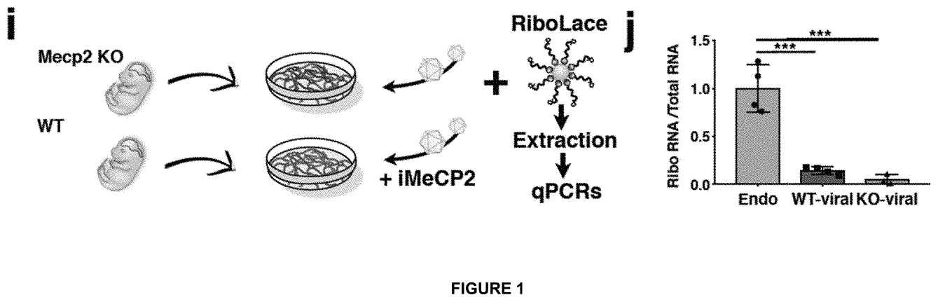

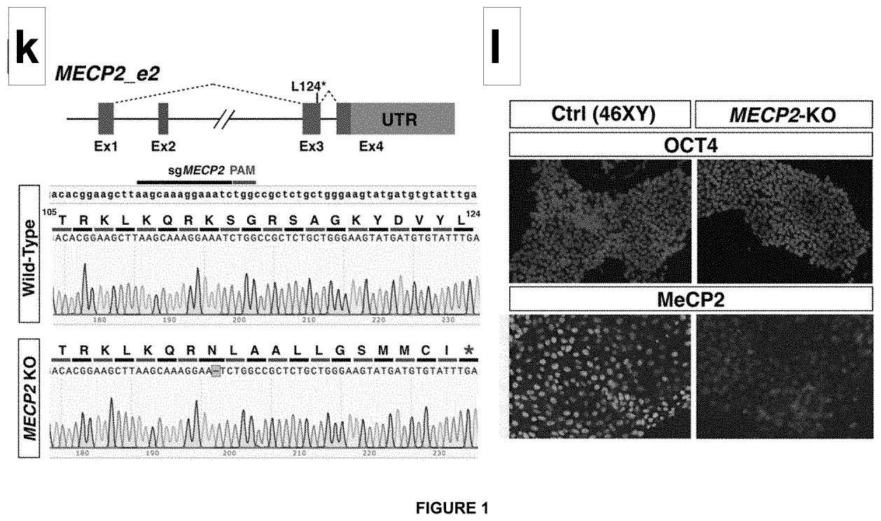

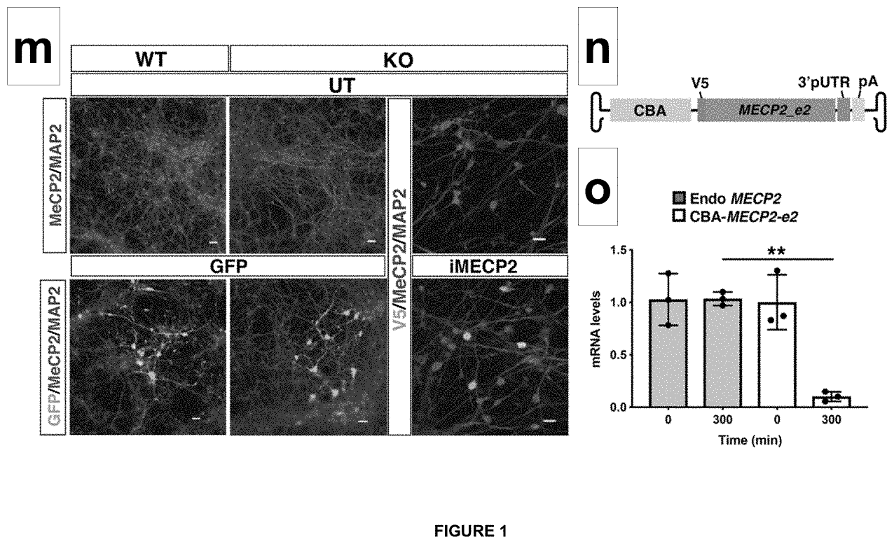

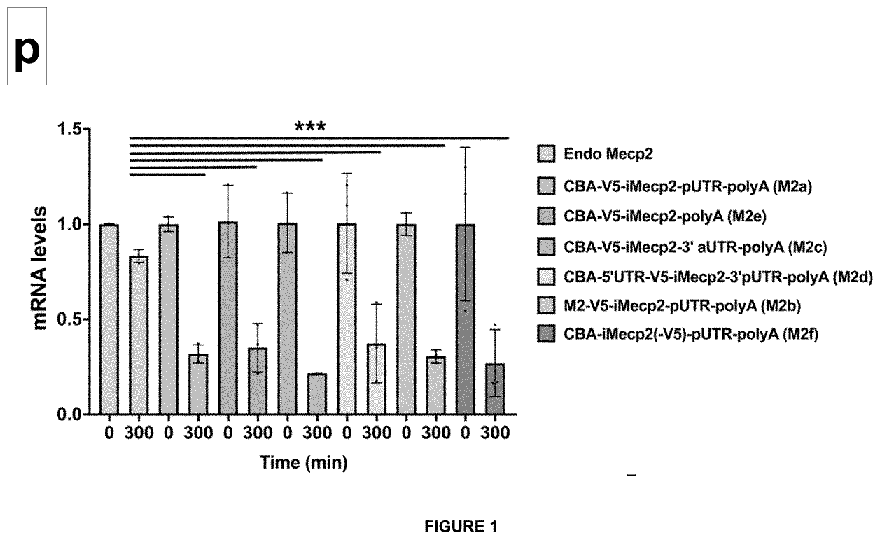

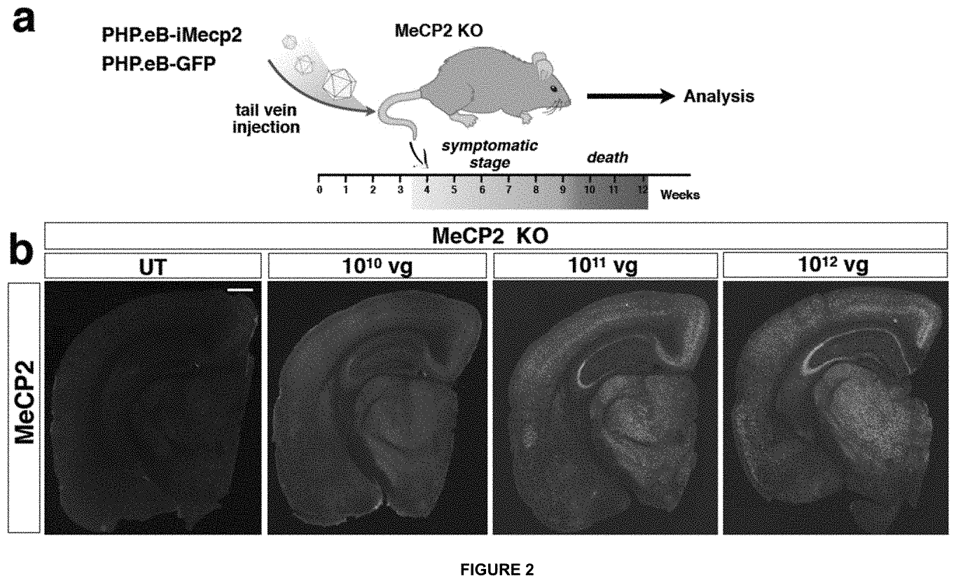

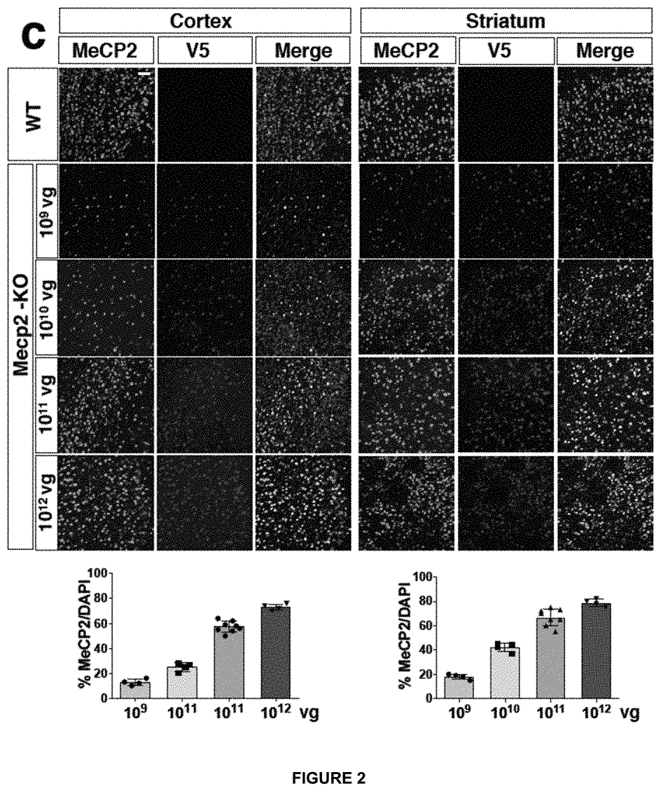

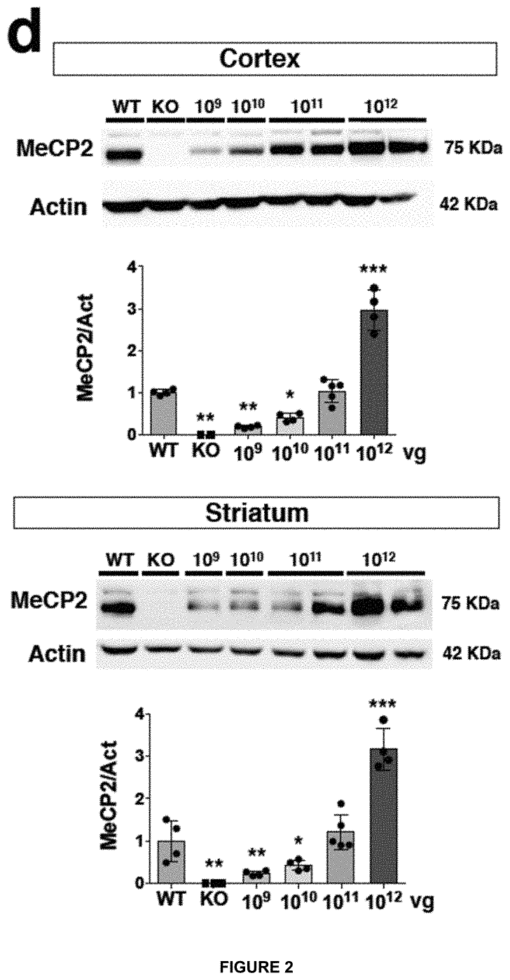

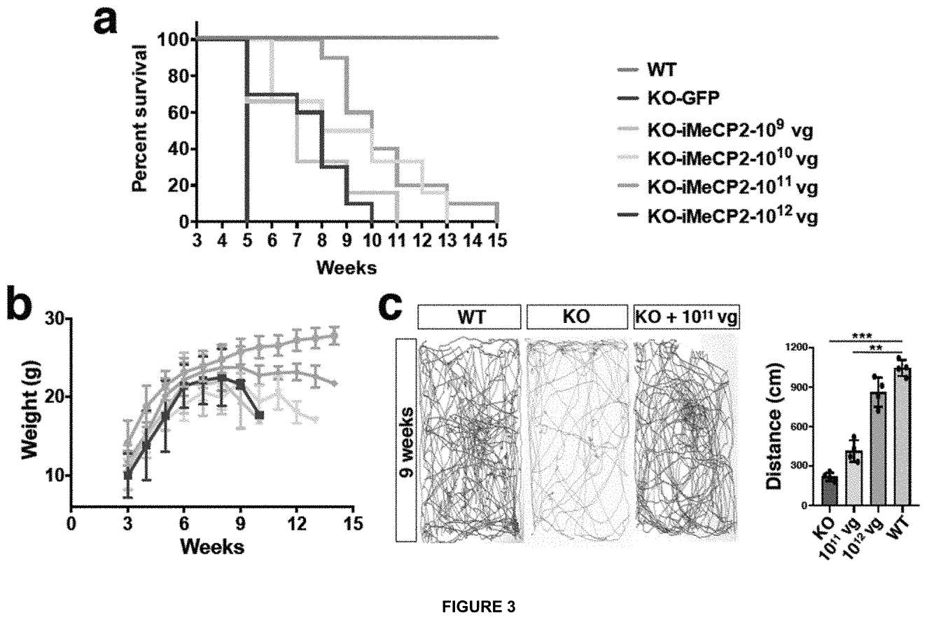

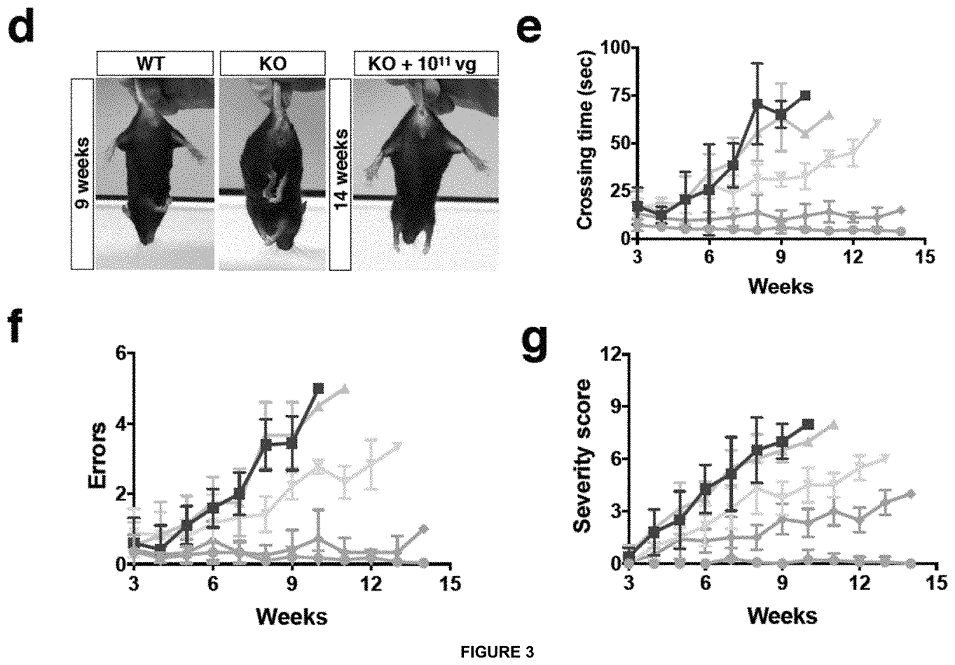

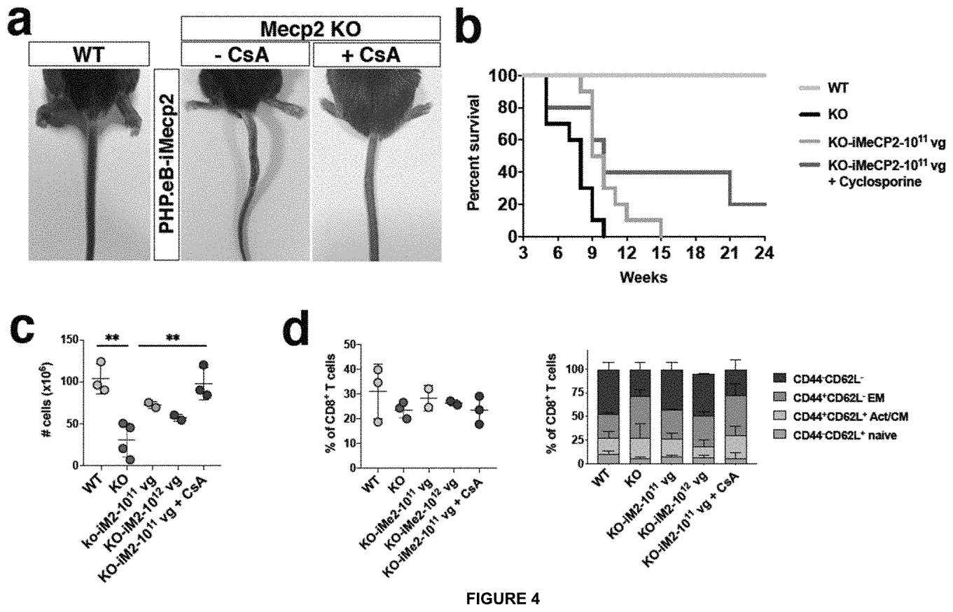

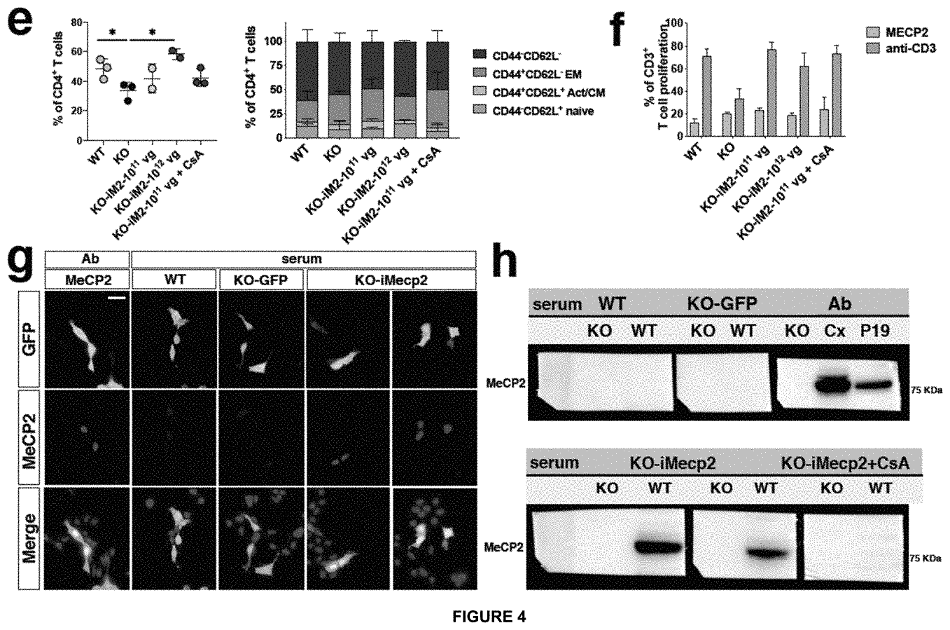

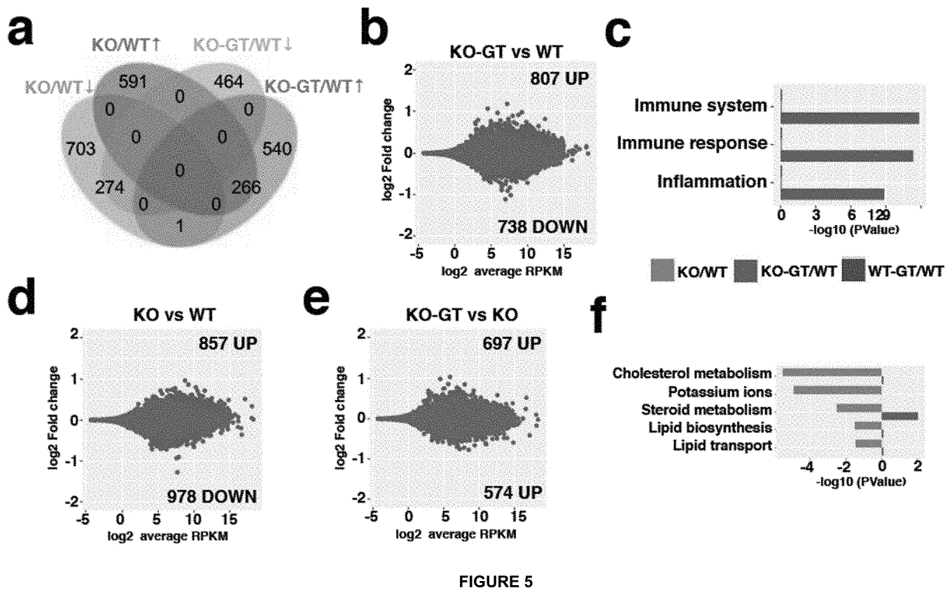

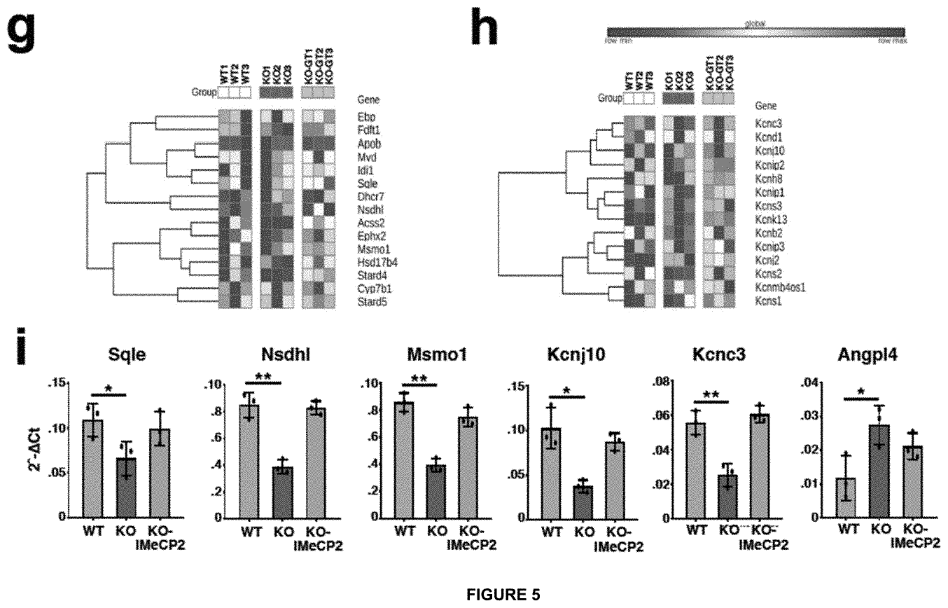

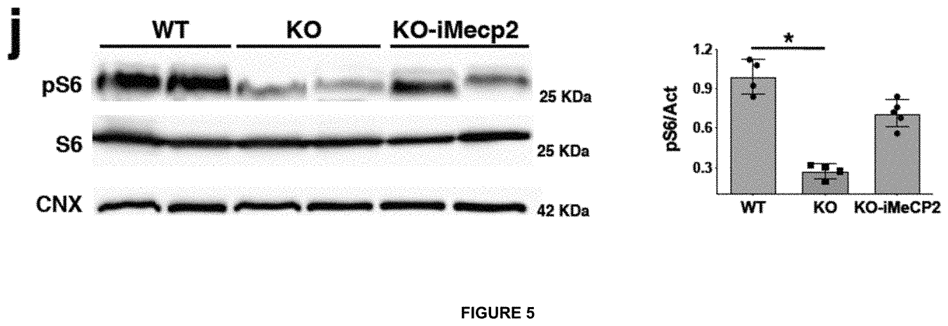

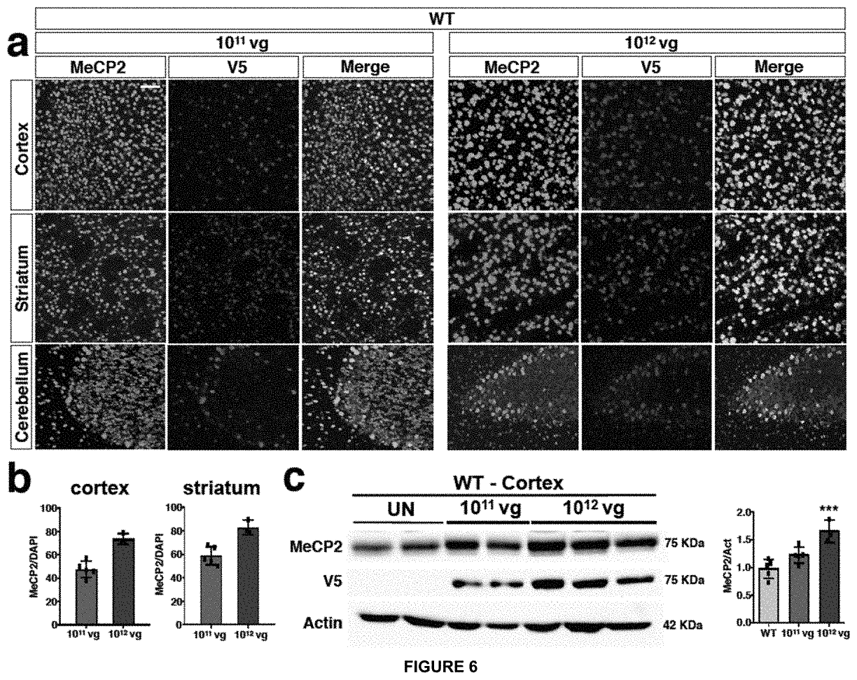

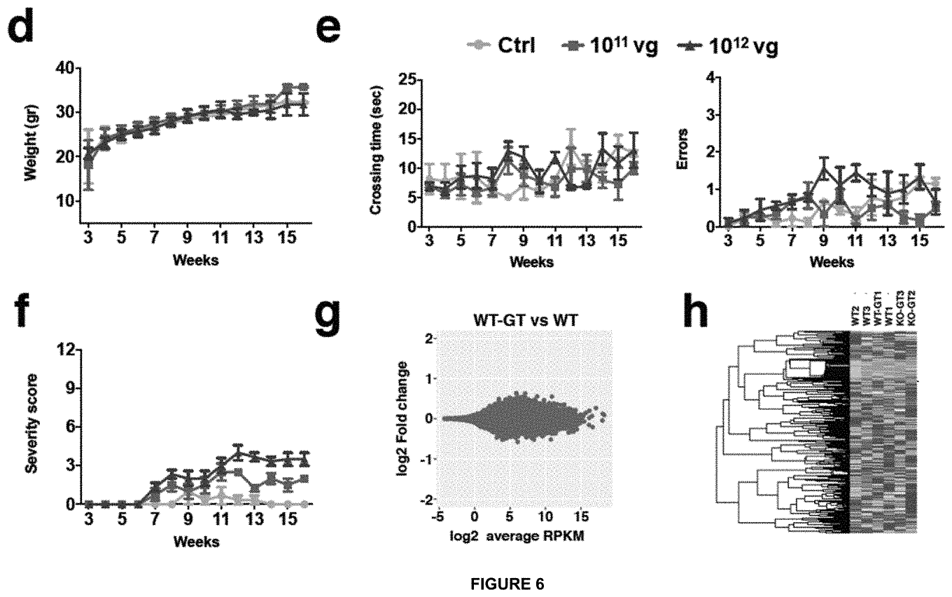

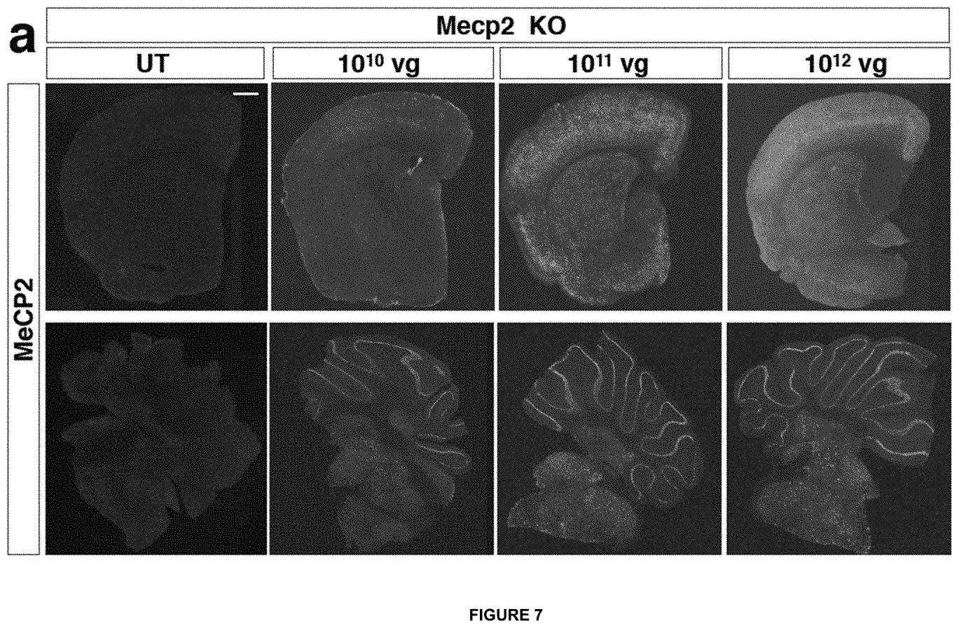

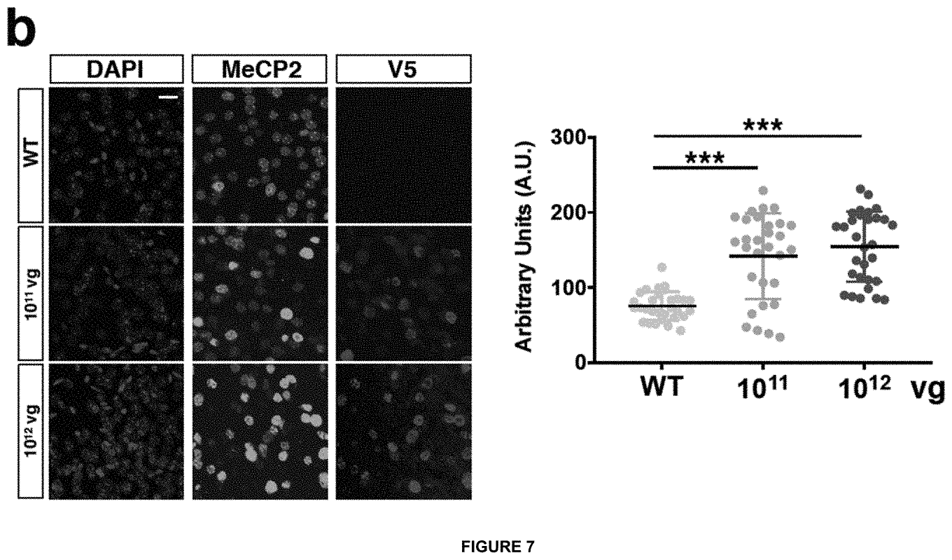

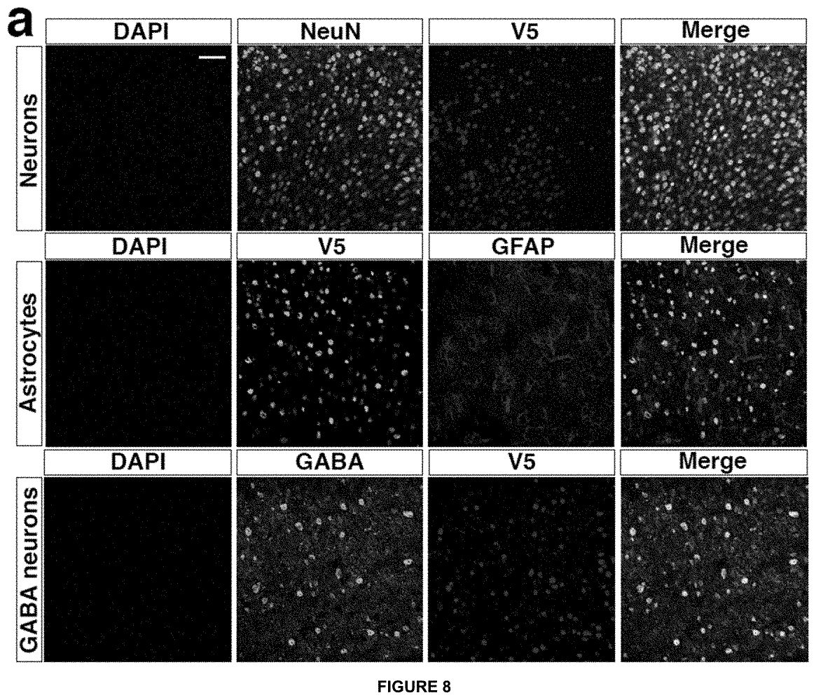

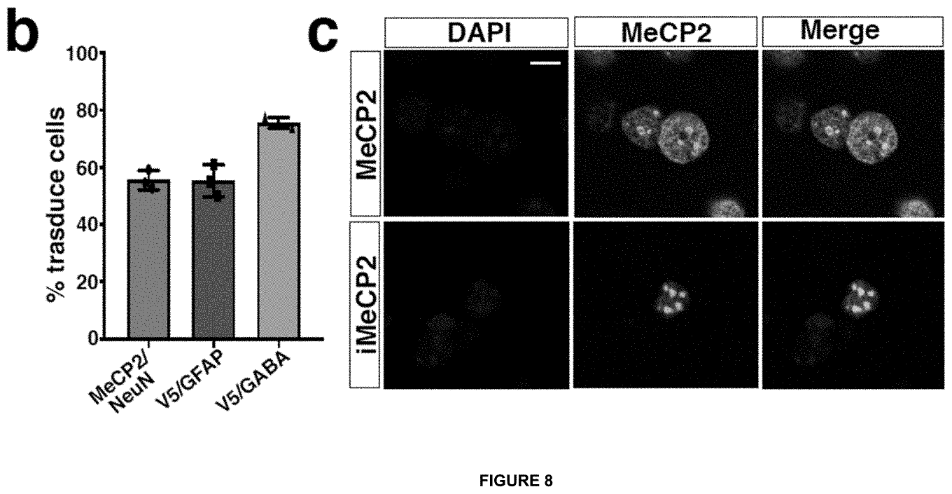

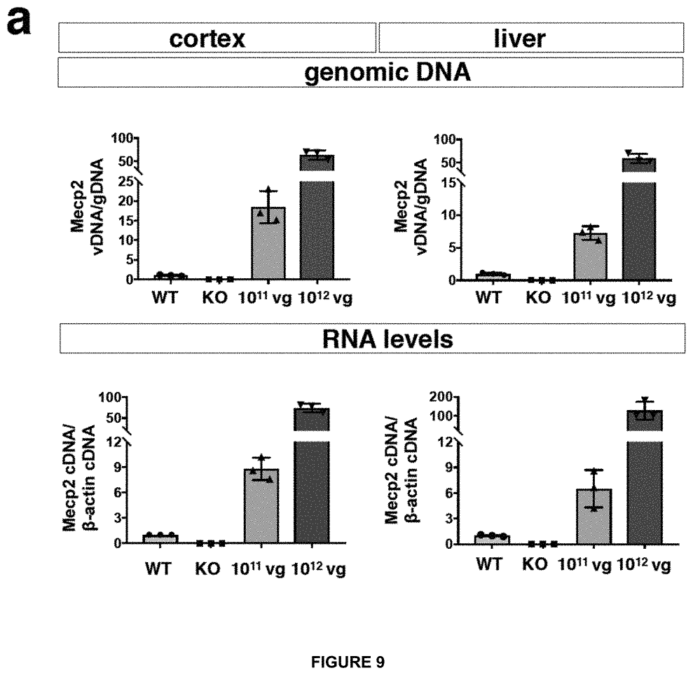

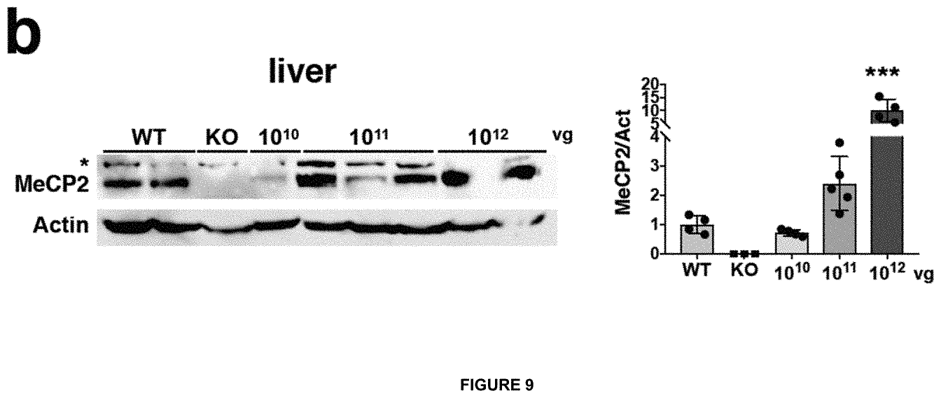

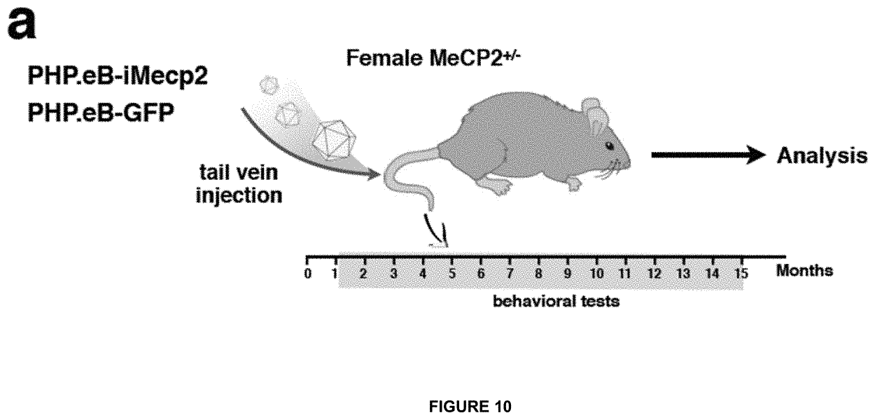

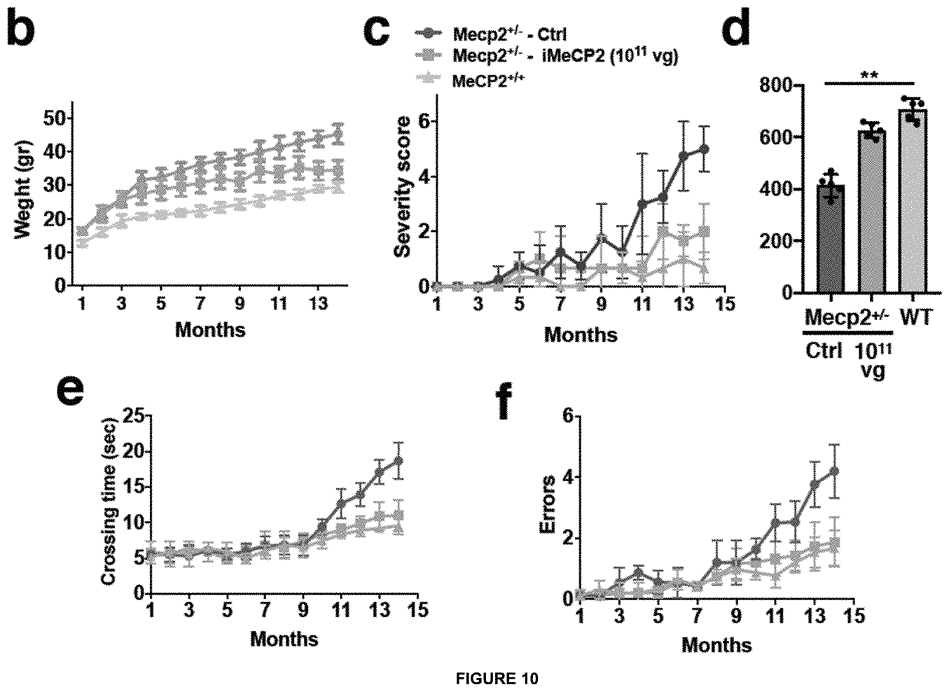

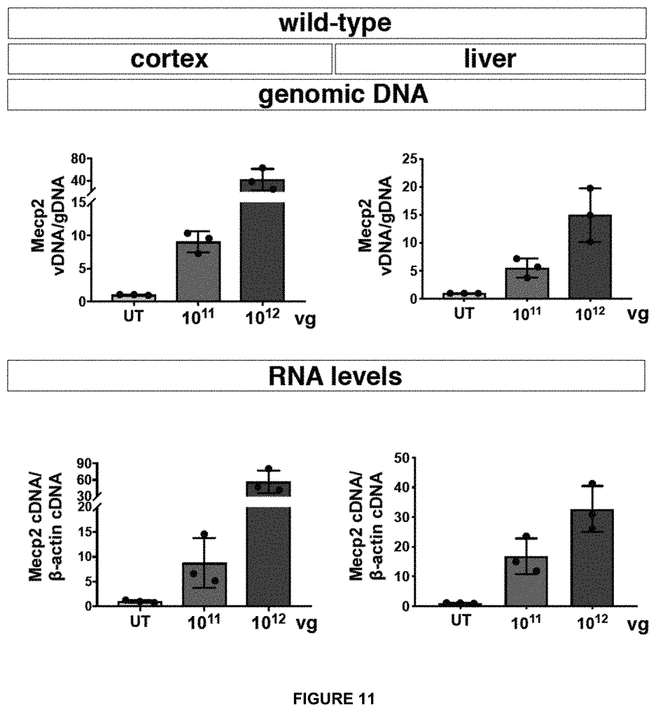

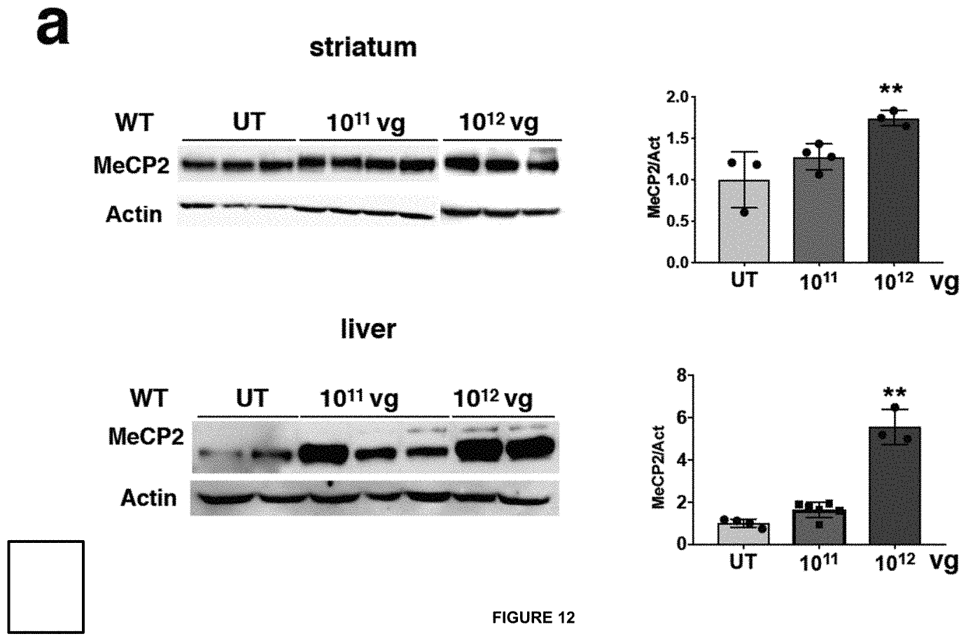

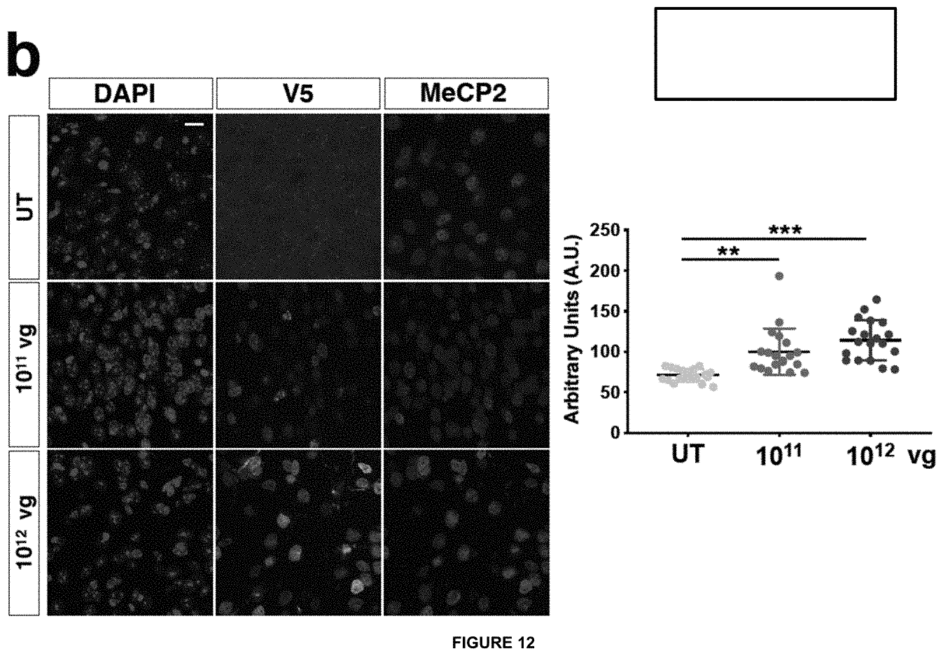

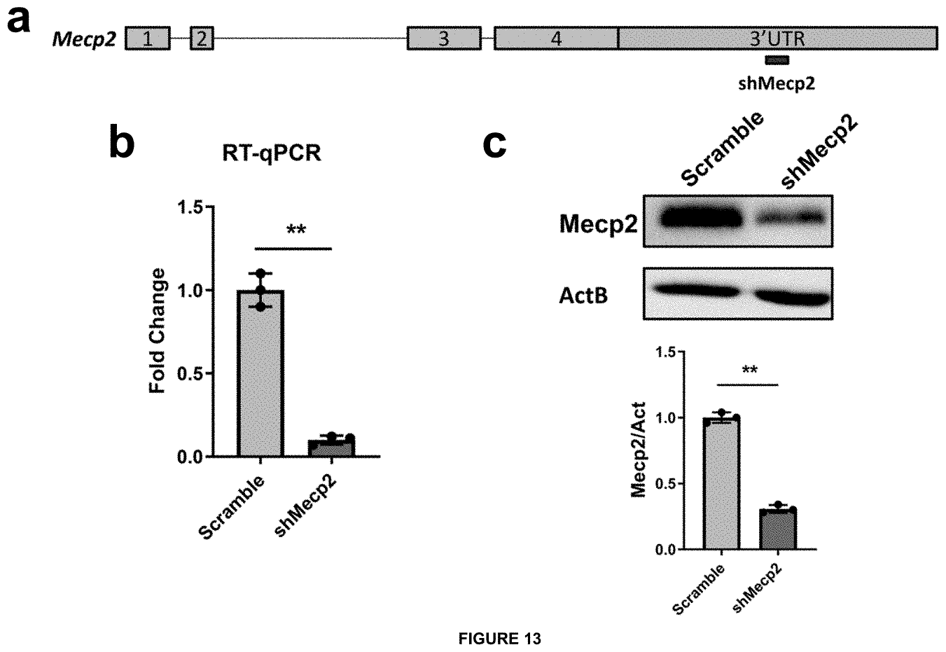

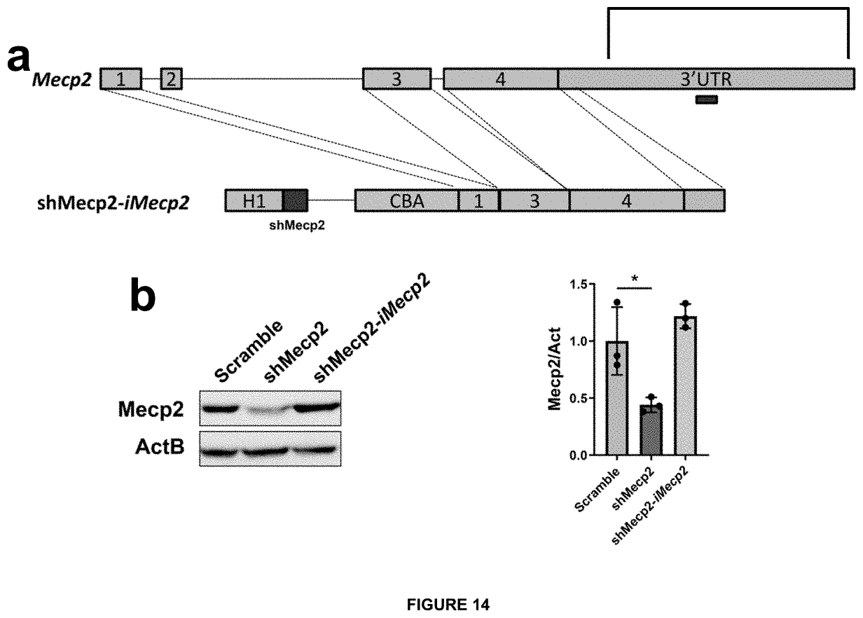

The Mecp2 transgene sequence carrying an N-terminal V5 tag was driven by two types of promoter (FIG. 1a). We used the chicken-beta-actin (CBA) promoter or alternatively we cloned a 1.4 kb fragment of the Mecp2 promoter with the intent to recapitulate the endogenous Mecp2 expression pattern. Mecp2^{-/y} primary neurons were infected with vector comprising either cassette, and two weeks later were lysed for immunoblotting and genome copy quantification.

Surprisingly, using the Mecp2 promoter fragment, the total viral MeCP2 protein amount remained significantly lower with respect to endogenous levels in transduced neurons (FIG. 1b). Conversely, mutant neurons infected with the CBA-Mecp2 cassette exhibited a physiological range of total MeCP2 protein. Moreover, similar results were obtained using MeCP2 immunofluorescence staining on infected neuronal cultures (FIG. 1c). This difference was not dependent on the relative infection load, since viral copy numbers were equivalent in neurons transduced with either of the two viruses (FIG. 1d).

Thus, of the two, only the CBA strong promoter was capable of re-establishing physiological MeCP2 protein levels.

This raised the question of why and through which mechanism the CBA-Mecp2 cassette could not exceed the endogenous Mecp2 protein range despite the presence of multiple copies of the virus in the neurons. We speculated that the lack of the UTR sequences from the Mecp2 transgene might intrinsically impair protein production. During neuronal development, Mecp2 transcripts with a long 3′-UTR are highly stabilised leading to progressive Mecp2 protein accumulation. In contrast, alternative Mecp2 isoforms with shorter 3′-UTRs are less stable and poorly regulated during development.

Thus, we sought to compare the relative stability of the viral Mecp2 transcript with respect to the total endogenous Mecp2 mRNA by measuring its half-life after gene transcription arrest with Actinomycin D (ActD) (FIG. 1e). Using the same qRT-PCR primers and reaction, viral Mecp2 transcripts were selectively amplified from PHP.eB-Mecp2 transduced neuronal cultures isolated from Mecp2 knock-out (KO) embryos, while total endogenous Mecp2 mRNAs were obtained from wild-type (WT) neuronal cultures (FIG. 1f).

Remarkably, viral Mecp2 transcripts showed significant lower RNA levels respect to the total endogenous Mecp2 transcripts after 300 min of ActD treatment (58%±4%) (FIG. 1f). Instability of the viral Mecp2 transcripts was comparable in Mecp2 KO and WT neuronal cultures (FIG. 1g). Likewise, stability of the endogenous Mecp2 mRNA was maintained unaltered in WT neurons infected or not with PHP.eB-Mecp2 (FIG. 1h). Thus, the lack of a long 3′-UTR generates an instability-prone Mecp2 (iMecp2) isoform which is significantly destabilised in neuronal cultures. To determine if this reduction in RNA half-life was only determined by the short UTR (pUTR), we generated an additional cassette with a synthetic assembled 3′-UTR (aUTR) which merged most of the known regulatory elements scattered in the 8.6 kb long 3′-UTR as previously reported (Matagne, V. et al. (2017) Neurobiol. Dis. 99: 1-11) (M2c, FIG. 1a).

In addition, we generated two more vectors, one including the physiological 5′-UTR sequence in combination with the pUTR sequence (M2d, FIG. 1a) and a second lacking the 3′-UTR thus carrying only the polyA (pA) sequence (M2e, FIG. 1a). Mecp2 RNA levels in neurons transduced with these viral vectors remained very unstable over time compared to endogenous mRNA levels (M2e: 48%±13%; M2c: 62%±0,2%; M2d 46%±20%; fragment of the Mecp2 promoter described above 53%±3%). Finally, we generated and tested a cassette lacking the V5 tag, to exclude a possible interference on the transgene mRNA stability (M2f, FIG. 1a), without observing significant differences in comparison with previous configurations (73%±18%, FIG. 1p).

Next, we sought to determine the translational efficiency of the viral iMecp2 variant. For this aim, we employed RiboLace, a methodology based on a puromycin-analogue which enables the isolation of the ribosomal fraction in active translation with their associated RNAs (Clamer, M. et al. (2018) Cell Reports 25: 1097-1108.e5). Thus, the translational active ribosomal fraction was captured by RiboLace-mediated pull-down from lysates of uninfected WT or PHP.eB-iMecp2 transduced Mecp2-KO neuronal cultures (FIG. 1i). Subsequently, mRNAs were extracted from both the isolated ribosomal fractions and the total lysates and used for RT-qPCR analysis with the same set of Mecp2 primers.

Remarkably, the normalised fraction of viral Mecp2 transcripts associated with translating ribosomes was reduced by 83%±5% compared with that of ribosome-bound endogenous Mecp2 mRNA (FIG. 1j). A similar reduction was calculated by assessing the relative fraction of ribosome-bound mRNAs of viral and endogenous Mecp2 in PHP.eB-iMecp2 infected WT neuronal cultures using isoform-specific set of primers (FIG. 1j).

Finally, we asked whether MECP2 RNA instability can represent a hurdle also in designing viral cassettes with the human MECP2 gene. Thus, we employed a pair of male isogenic iPSC lines either as a control or with a CRISPR/Cas9 induced MECP2 loss-of-function mutation (FIG. 1k,l). Both iPSC lines were differentiated in vitro into cortical neuronal cultures and, then, control and MECP2 mutant lines were transduced with AAV expressing either GFP or the human version of iMECP2, respectively (FIG. 1m,n). Noteworthy, viral MECP2 transcript stability was severely affected as compared to endogenous RNA levels to an extent comparable to what observed with murine mRNAs (FIG. 10).

In summary, these data revealed the previously unknown dominant effect of the post-transcriptional processes to determine the final output of the viral Mecp2 protein levels. Given this limited efficiency in transcript stability and translation efficacy only the use of the CBA strong promoter was effective to sustain Mecp2 protein levels comparable to those found in WT primary neurons.

PHP.eB-iMecp2 Treatment of Male Mecp2 Mutant Mice

Gene Transfer

To test the efficacy of gene transfer with the iMecp2 transgene cassette we designed a dose escalation approach to administer 10-fold increasing doses of PHP.eB-iMecp2 (1×10⁹ vg, 1×10¹⁰ vg, 1×10¹¹ vg and 1×10¹² vg/mouse) of virus through intravenous delivery in 4 weeks old Mecp2^{-/y} mice and untreated Mecp2^{-/y} animals were utilised as controls (FIG. 2a, FIG. 7a). To determine the exact brain penetration efficiency and neural tissue transduction of the PHP.eB-iMecp2, three mice for each viral dose were euthanised and brains separated in two halves for immunohistochemistry and immunoblot analysis twenty days after administration. Sections of the infected Mecp2 mutant brains were stained for Mecp2 or V5 to visualise the viral Mecp2 transduction pattern.

The distribution of PHP.eB-iMecp2 was spread throughout the brain with increasing intensity using higher viral doses (FIG. 2b), as also measured with fluorescence intensity (FIG. 7b). Quantification of Mecp2⁺ cells with respect to DAPI⁺ nuclei in the somatosensory cortex and striatum showed a proportional increase in transduction efficiency from the lowest (1×10⁹ vg; 15±3% in cortex; 18±3% in striatum) to the highest dose (1×10¹² vg; 78±3% in cortex; 80±4% in striatum). Brain tissue transduced with 10¹¹ vg of PHP.eB-iMecp2 and immunodecorated for sub-type cellular markers showed that viral transduction was equally efficient in infecting both neuronal and glial cells (FIG. 8a,b). In particular, the cortical GABAergic interneurons whose dysfunction is a crucial determinant of the RTT phenotype were effectively transduced (FIG. 8a,b). Sub-cellular analysis of viral Mecp2 protein distribution confirmed a strong enrichment in nuclear heterochromatic foci, mirroring the genome-wide distribution of endogenous Mecp2 (FIG. 8c).

Subsequent Western blot analysis showed increasing total levels of Mecp2 protein in cortical and striatal tissues upon transduction with higher doses of PHP.eB-iMecp2 (FIG. 2d). In particular, administration of 10¹¹ vg of PHP.eB-iMecp2 resulted in Mecp2 protein levels comparable to those detectable in control brains (FIG. 2d). Conversely, 10¹² vg of PHP.eB-iMecp2 triggered a 3-fold increase in Mecp2 expression respect to endogenous levels (FIG. 2d).

To further assess the efficiency of viral transduction, we measured the number of viral iMecp2 copies present in the brain and liver of the treated mice. PHP.eB-iMecp2 treated animals showed a higher number of viral copies in the brain respect to the liver (brain: 15±5, 10¹¹ vg; 65±15, 10¹² vg. liver: 7±2, 10¹¹ vg; 55±15, 10¹² vg) (FIG. 9) confirming the higher propensity of transducing the neural tissue respect to peripheral organs of the PHP.eB capsid. In contrast, transgene RNA levels were proportionally less abundant compared to the relative viral genome copy in brain with respect to the liver (FIG. 9). In addition, despite the significant increase in iMecp2 genomic copies and total mRNA, protein levels were only marginally augmented in brain and partially in liver (FIG. 10). This effect was particularly evident with the 10¹² vg dose which triggered 80%±35% increase in mRNA with only a 3-fold protein increase. These observations confirmed that iMecp2 mRNA is poorly transcribed and translated in brain tissue as previously shown in neuronal cell cultures.

Altogether, these data clearly highlight the robust efficiency of the PHP.eB capsid to cross the blood-brain barrier in adult mouse brains and to spread throughout the neural tissue transducing large number of cells. Importantly, the four doses of PHP.eB-iMecp2 which differed by a 10-fold higher titer showed a proportional increase in transduction efficiency in the brain. Hence, this escalation in viral transduction offered a great opportunity to test the extent of phenotypic rescue in Mecp2 mutant mice depending by the viral gene transfer efficiency and the relative number of cells with restored Mecp2 expression.

Behavioural Response

Next, we treated mice with increasing doses of PHP.eB-iMecp2 (1×10⁹ vg, 1×10¹⁹ vg, 1×10¹¹ vg and 1×10¹² vg) and their control littermates (WT and GFP treated Mecp2^{-/y} mice, 1×10¹¹ vg). Four weeks old Mecp2^{-/y} mice were intravenously injected and examined over time to monitor the progression of behavioural deficits and the relative efficacy of the treatments. As previously reported, Mecp2^{-/y} mice in a C57BI/6 background have reduced body size and are lighter compared to their WT littermates and between 9-11 weeks of age they experience a sudden weight loss which anticipates the worsening of RTT symptoms and their following decease (FIG. 3a,b). Animals were euthanised right before this stage for ethical reasons.

A similar lifespan length was observed in mice administered with two different doses of control PHP.eB-GFP virus (10¹¹ vg and 10¹² vg). Conversely, the Mecp2^{-/y} mice treated with 10¹⁰ vg and 10¹¹ vg of PHP.eB-iMecp2 showed a significant weight gain over the following weeks with a significant increase in lifespan reaching a survival median period of 59d and 68d, respectively (FIG. 3a,b).

To determine the general symptomatic stage, the animals were subjected to a battery of locomotor tests and the total grading for inertia, gait, hindlimb clasping, tremor, irregular breathing and poor general conditions that together were presented as the aggregate severity score. Mecp2^{-/y} mice treated with the median viral doses (10¹⁰ vg and 10¹¹ vg) maintained pronounced locomotor activity and exploratory behavior as assessed in the open field until few days before their sacrifice (FIG. 3c). Indeed, most of mice treated with 10¹¹ vg particles were euthanised even if symptoms were not such to require sacrifice, but because of a severe tail necrosis unrelated to the disease phenotype. This group of mice did not show any sign of hindlimb clasping (FIG. 3d), as well as mice treated with lower dosage (10¹⁰ vg). The Mecp2^{-/y} mice injected with 10¹¹ vg of therapeutic virus maintained high motor behaviour skills with a number of errors and crossing time through a beam similar to control treated animals (FIG. 3 e,f). Additionally, this group of mice never exhibited high grades in the aggregate severity score (FIG. 3g). Mecp2 deficient mice treated with 10¹⁰ vg of PHP.eB-iMecp2 also exhibited a mild, although still significant, rescue of motor functions and the general symptomatic phenotype (FIG. 3e-g). In summary, this phenotyping assessment showed that the 10¹¹ vg dose of PHP.eB-iMecp2 elicited a robust and long-lasting recovery in survival and behavioral skills. A phenotypic improvement in Mecp2 deficient mice was also elicited with a 10-fold reduced dose of the therapeutic virus (10¹⁰ vg) although with a reduced rescue.

The aforementioned immunofluorescence analysis showed a proportional relationship between the increasing doses of virus inoculated in the animals and the enhanced Mecp2 gene transfer in the brain. With these data we can conclude that below 15% of efficiency in brain (cortex and striatum) transduction the Mecp2^{-/y} pathological deficits remain mostly irreversible. Alternatively, a transduction rate between 20-30% is sufficient to sustain some detectable behavioural improvement. Finally, restoring Mecp2 expression in at least 70% of the brain cells exerts a strong and sustained amelioration of the RTT pathology.

Immune Response

PHP.eB-iMecp2 and not the control virus triggered severe bruised skin and tail with profound ulcers in the treated mice that are not common manifestations in RTT (FIG. 4a). We hypothesized that the lack of Treg-mediated regulation was responsible for the strong immune response observed in Mecp2^{-/y} mice exposed to PHP.eB-iMecp2.

Treatment with cyclosporine (CsA) of Mecp2^{-/y} mice exposed to a 10¹¹ vg dose of PHP.eB-iMecp2 resulted in the increased of lifespan in 5 out of 9 treated mice (FIG. 4b) and in a striking amelioration of the disease phenotype.

The analysis of the frequency of cells in the spleen of Mecp2^{-/y} mice exposed to 10¹¹ vg and 10¹² vg of PHP.eB-iMecp2 (KO-iMecp2-10¹¹ vg and KO-iMecp2-10¹² vg, respectively) showed the increased number of splenocytes harvested from the latter mice compare to untreated Mecp2^{-/y} controls (FIG. 4c,d). Interestingly, Mecp2^{-/y} mice showed a significantly lower number of splenocyte compared to WT littermates, in line with the general status of inflammation due to spontaneous activation of T cells in these mice. No major differences were observed in CD8⁺ T cell compartments in Mecp2^{-/y} mice untreated or exposed to 10¹¹ vg and 10¹² vg of PHP.eB-iMecp2 virus (FIG. 4d). Interestingly, Mecp2^{-/y} mice exposed to the higher PHP.eB-iMecp2 dose (10¹² vg), analysed two weeks post treatment, showed a significantly higher frequency of CD4+ T cells compared to untreated control mice (FIG. 4e). Hence, we can speculate that treatment with the higher dose of PHP.eB-iMecp2 virus in Mecp2^{-/y} mice lacking Treg-mediated regulation led to an uncontrolled inflammatory response associated to the expansion of CD4⁺ T cells.

We then investigated the induction of Mecp2-specific immune response by analysing proliferation of T cells in response to Mecp2, cytotoxic activity of CD8⁺ T cells, and anti-Mecp2 antibody production in Mecp2^{-/y} mice exposed to PHP.eB-iMecp2 virus. Neither Mecp2-specific T cells nor Mecp2-specific cytotoxic CD8+ T cells were detected in Mecp2^{-/y} mice exposed to PHP.eB-iMecp2 virus (FIG. 4f). However, an increased non-specific (anti-CD3-stimulated cells) proliferative response was observed in Mecp2^{-/y} mice exposed to PHP.eB-iMecp2 virus compared to untreated mice (FIG. 4f).

Finally, we detected anti-MeCP2 antibodies in the sera of Mecp2^{-/y} mice exposed to PHP.eB-iMecp2 virus but not in WT mice exposed to PHP.eB-iMecp2 virus or in control untreated Mecp2^{-/y} mice (FIG. 4g,h). Importantly, treatment with CsA prevented the induction of anti-Mecp2 antibodies (FIG. 4h).

Overall, these studies indicate, for the first time, the induction of uncontrolled proliferation of T cells and induction of Mecp2-specific antibodies in Mecp2^{-/y} mice exposed to PHP.eB-iMecp2 virus, which can be overcome by immunosuppression. This severe immune response to the transgene can explain the premature death of Mecp2^{-/y} mice treated with the highest dose of the therapeutic virus (10¹² vg). This conclusion is corroborated by the fact that WT animals exposed to the same dose virus at a comparable dose did not develop any of these complications (see below).

Molecular and Gene Expression

In order to examine the molecular alterations downstream to Mecp2 loss and evaluate whether gene therapy with PHP.eB-iMecp2 might sustain any discernable recovery, we performed global gene expression analysis by RNA-Seq of whole cerebral cortical tissue from 9 weeks old Mecp2^{-/y} mice inoculated with either 10¹¹ vg of PHP.eB-iMecp2 or control virus and WT littermates.

Computational analysis identified 1876 differential expressed genes (DEGs) with p<0.05 significance between Mecp2^{-/y} and control mice roughly divided in two equal groups between up- and down-regulated genes in mutant mice. The number of significant DEGs between Mecp2^{-/y} mice administrated with therapeutic or control virus was 1271 with a small increase in upregulated genes. However, only a third of DEGs were shared between viral transduced and untreated Mecp2^{-/y} mice, while the remaining DEGs of the mutant mice were normalised in the treated counterparts.

This data suggested that the viral treatment was able to correct a large fraction of gene expression changes associated with the RTT phenotype. However, a large set of DEGs was only associated with the treated Mecp2^{-/y} mice and, thus, to uncover their significance we performed Gene Ontology functional enrichment analysis (GO).

Remarkably, most of these DEGs were associated with immunological pathways such as immune response, immune system regulation and inflammation (FIG. 5c). Hence, these results corroborated at the molecular level the previous observations on the strong immune response mounted in the treated Mecp2^{-/y} mice against the inoculated transgene.

We, then, performed gene ontology analysis on the DEG dataset enriched in the Mecp2^{-/y} mice but normalised after gene therapy.

Interestingly, the most significant enrichment was in metabolic networks associated with lipid biosynthesis/transport and in particular cholesterol metabolism (FIG. 5f). Remarkably, a large component of the molecular pathway for cholesterol production was downregulated in Mecp2^{-/y} mice, but significantly rescued in PHP.eB-iMecp2 transduced animals (FIG. 5g). RT-qPCRs on independent cortical tissue lysates confirmed that gene expression levels of crucial enzymes in the cholesterol biosynthesis such as Squalene epoxidase (Sqle), NAD(P)-dependent steroid deydrogenease-like (Nsdhl) and Methylsterol monooxygenase 1 (Msmo1) were significantly restored by PHP.eB-iMecp2 gene therapy (FIG. 5i).

An additional molecular group highly divergent between transduced and control Mecp2^{-/y} mice included genes encoding for potassium (Kv) channels. This class of ion channels serve diverse functions including regulating neuronal excitability, action potential waveform, cell volume and fluid and pH balance regulation. We confirmed reduced Kcnj10 transcripts together with gene deregulation of other Kv channels in Mecp2^{-/y} mice (FIG. 5h,i). Among others, the potassium channel gene Kcnc3, associated with ataxia and cognitive delay in humans, was downregulated in Mecp2 mutants and normalised after the PHP.eB-iMecp2 treatment (FIG. 5h,i). Remarkably, overall levels of pS6 on Ser234/235 were significantly increased in brain tissue transduced with the PHP.eB-iMecp2 virus (FIG. 5j).

In summary, PHP.eB-iMecp2 gene therapy sustained a wide recovery of the abnormal gene expression in the Mecp2 mutant brain tissue and elicited the rescue of the global impairment affecting transcriptional and translational processes upon Mecp2 gene loss.

PHP.eB-iMecp2 Treatment of Female Mecp2 Heterozygous Mice

Mecp2^{-/y} mice are an extremely severe model of RTT and do not reflect the mosaic gene inactivation occurring in girls with RTT. Thus, we thought to validate our approach in female Mecp2^{+/-} mutant mice.

To this end, 3-months old Mecp2^{+/-} animals were intravenously injected with 10¹¹ vg of either PHP.eB-iMecp2 or control virus and examined over time up to 11 months post-treatment (FIG. 10a). For these experiments, we selected only a single viral dose that triggered the best recovery in Mecp2^{-/y} mice.

As previously reported, Mecp2^{+/-} females started to exhibit pathological signs from 10 months of age acquiring breathing irregularities, ungroomed coat, inertia and hindlimb clasping (Guy, J. et al. (2001) Nature Genetics 27: 322-326). While control treated mutant females showed a pronounced and sustained weight gain over time, the animals with the viral therapy gradually normalised their weight reaching values similar to those of unaffected mice at 15 months (FIG. 10b). In the severity score the control treated Mecp2^{+/-} females progressed to values over 4, whereas animals given the therapeutic virus rarely overcome a score beyond 2 in the entire observation period (FIG. 10c). Total mobility assessment in the open field showed that at 13 months old PHP.eB-iMecp2 treated mice have a significant increase in the travelled distance and general activity respect to the control treated group matching the general performance of WT females (FIG. 10d). Likewise, in the beam balance test which assesses fine motor movements and coordination, the therapy largely preserved the motor skills of the Mecp2^{+/-} mice that, in contrast, were partially lost in control mutant mice (FIG. 10e,f).

Collectively, these observations provide evidence that the PHP.eB-iMecp2 treatment sustained a significant and long-term protection from symptomatic deterioration improving the health conditions and reducing the locomotor phenotype in female Mecp2^{+/-} mice.

Systemic Gene Transfer of iMecp2

Systemic delivery of PHP.eB-iMecp2 exerted a large symptomatic reversibility both in male and female Mecp2 mutant mice. To further extend these observations and validate the safety of this treatment we decided to administer the same treatment to WT C57BL/6 adult mice.

Animals were administrated with either 10¹¹ vg or 10¹² vg of PHP.eB-iMecp2 or left untreated (n=9 each) and closely inspected over time. Next, two animals per group were euthanised 3 weeks after viral inoculation and brain processed for histological analysis. iMecp2 gene transfer efficiency was evaluated by V5 immunofluorescence which distinguished the viral from the endogenous Mecp2.

According to aforementioned results, brain transduction efficiency was very high with a net increase of 20% between the lower and the higher viral dose (cortex: 45%±8% 10¹¹ vg, 68%±7% 10¹² vg; striatum: 58%±7%, 10¹¹ vg; 82%±5%, 10¹² vg) (FIG. 6a,b). Viral copy number analysis confirmed a significant and prevalent targeting of the virus in the neural tissues with respect to the liver (FIG. 11).

Despite the high expression of the transgene, the total Mecp2 protein levels in cortex and striatum were only increased by 30% and 60% upon transduction with 10¹¹ vg and 10¹² vg of virus, respectively as assessed by immunoblotting and immunofluorescence intensity (FIG. 6c, FIG. 12).

General health state and behaviour were then scored in the remaining treated animals up to 12 weeks after treatment. During this time, growth curve, locomotor activity and fine coordination were slightly different between transduced and control animals (FIG. 6d,e). General health state examination (severity score) revealed some breathing irregularities and tremor at rest which slightly increased the scoring output in the treated animals although with minimal difference compared to WT animals (FIG. 6f).

Altogether, these observations indicated that high doses of PHP.eB-iMecp2 virus did not exert deleterious effects in WT animals in this window of time. Importantly, even the 10¹² vg dose, which is 10-fold higher than the amount used in Mecp2^{-/y} mice to trigger substantial beneficial effects, was incapable of triggering a consistent deleterious outcome in the mice except for mild alterations.

Next, we performed global gene-expression analysis by RNA-seq from cerebral cortical tissues of animals untreated or inoculated with a 10¹² vg dose. Remarkably, bioinformatics analysis did not distinguish genes with significantly different expression between the two conditions (p<0.05) (FIG. 6g,h). Collectively, widespread brain transduction of PHP.eB-iMecp2 in WT animals elicited only a minimal increase in total Mecp2 levels which was not sufficient to exert either significant behavioural symptoms or abnormal gene expression changes.

### CONCLUSION

Herein, we provide evidence that the global brain transduction of the iMecp2 transgene by PHP.eB-mediated delivery is capable of significantly protecting male and female Mecp2 mutant mice from the symptomatic hallmarks of the RTT phenotype.

Our results showed that the lack of a long 3′-UTR sequence destabilises Mecp2 mRNA significantly reducing its relative half-time. Additionally, using the RiboLace system to determine the amount of Mecp2 transcripts associated with active translating ribosomes, our data showed a major loss in translation efficiency of the viral compared to the endogenous mRNA. In addition to the 3′-UTR, the 5′-UTR sequence also has a fundamental control on MeCP2 protein synthesis and its impairment leads to RTT (Saxena, A (2006) J Med Genet 43: 470-477). Our results emphasise the critical importance of the role played by the UTR sequences on the viral transgene function.

We combined the Mecp2 cDNA with the CBA strong promoter, building the iMecp2 cassette, in order to reach physiological levels of Mecp2 proteins. More broadly, a preliminary in vitro screening through PHP.eB-mediated transduction of primary neuronal cultures is valuable to determine the total protein levels achievable with any newly designed transgene cassette and draw a direct comparison with protein amounts of the endogenous gene copy.

We decided to package the iMecp2 construct in the PHP.eB that was selected for its unprecedented capability to efficiently penetrate the brain after intravenous delivery and widely transduce neuronal and glial cells (Chan, K. Y. et al. (2017) Engineered AAVs for efficient noninvasive gene delivery to the central and peripheral nervous systems. Nature Publishing Group 1-17 (2017)). We showed that administration of high dose of PHP.eB-iMecp2 at the initial symptomatic stage can ameliorate disease progression with robust beneficial effects and significant lifespan extension. It was unanticipated that a relatively small fraction of reconstituted cells could sustain appreciable beneficial effects. This may reflect redundancy of cells in behavioural circuits or other compensatory mechanisms to sustain neuronal circuitries with insufficient Mecp2. On this perspective, PHP.eB-iMecp2 transduced Mecp2^{-/y} presenting increasing cellular fractions with restored Mecp2 might represent an unprecedented model to study circuitry function and its dependency by Mecp2.

The only detrimental effect we encountered was the strong immune response in mutant males that was controlled by administration of cyclosporin. This response uncontrolled inflammatory response to the transgene, associated to the expansion of CD4⁺ T cells, can explain the sudden death of the Mecp2^{-/y} animals treated with the highest PHP.eB-iMecp2 dose (10¹² vg). In fact, a similar dose of the therapeutic virus in WT animals was free of severe deleterious effect. Importantly, this adverse complication is the result of using full knock-out male Mecp2 mice that have no Mecp2 from birth and therefore recognise the therapeutic gene product as nonself. This is not the case for human patients that are a mosaic of mutant and WT Mecp2 cells and, thus, will not mount any immune response against this transgene.

Beyond this immune reaction, we could not score any additional adverse manifestations directly caused by the therapy neither in mutant Mecp2 or WT animals. Nevertheless, the mild increase in total Mecp2 levels was able to promote a robust improvement in locomotor activity and coordination, general health state and survival. In addition, PHP.eB-iMecp2 intervention significantly corrected the abnormal gene expression alterations observed in Mecp2^{-/y} mouse cortical tissue including the reduced expression in multiple molecular components of the cholesterol pathway and some genes for Kv channels with crucial functions for astrocytes and neurons. More broadly, PHP.eB-iMecp2 gene therapy sustained a strong recovery of the genome-wide transcriptional and mTOR-mediated translational processes affected in Mecp2 deficient mice.

Taken together, the PHP.eB-iMecp2 treatment can be considered a very effective and safe therapeutic strategy to counteract disease progression and ameliorate symptomatic manifestations. The diffuse penetration of the PHP.eB in the adult mouse brain parenchyma is a unique property among all the recombinant viral strains in current use. The PHP.B/eB receptor belongs to the large family of Ly6/uPar proteins, some of which are conserved in mammalian evolution and can be found in human brain endothelium, representing valuable targets for capsid engineering (Loughner, C. L. et al. (2016) Human Genomics 1-19 doi:10.1186/s40246-016-0074-2). Thus, the PHP.B platform may be used to test the validity of new gene therapy strategies in mice models that can be translated into the clinical setting.

RTT is a neurodevelopmental disorder and the therapeutic intervention should be finalised in the first months after birth as soon as the early signs of the disease manifest and genetic diagnosis is certain. At similar age, an intravenous infusion of AAV9 particles packaging the SMN1 gene resulted in extended survival and improved motor functions in infants suffering for spinal muscular atrophy (Mendell, J. R. et al. (2017) N. Engl. J. Med. 377, 1713-1722). Thus, it is plausible that at this early age, AAV9 systemic gene therapy will be sufficient to sustain a beneficial clinical outcome in RTT patients.

Overall, this study provided a new iMecp2 viral cassette with improved safety and efficacy for the symptomatic amelioration in faithful animal models of RTT.

Materials and Methods

Animals

Mice were maintained at San Raffaele Scientific Institute Institutional mouse facility (Milan, Italy) in micro-isolators under sterile conditions and supplied with autoclaved food and water. The Mecp2⁴ mice were maintained on C57BL/6 background. All procedures were performed according to protocols approved by the internal IACUC and reported to the Italian Ministry of Health according to the European Communities Council Directive 2010/63/EU.

Generation of Gene Transfer Vectors

The murine Mecp2 isoform 1 (NM_001081979.2) CDS including 3′-UTR (223 bp) was PCR amplified in order to add the V5 tag at the 5′ of the coding sequence and inserted in the CBA-CreNLS vector (Morabito, G. et al. (2017) Molecular Therapy 25: 2727-2742) to generate the iMecp2 vector. The AAV-CBA-V5-GFP construct was engineered from AAV-CBA-V5-Mecp2 vectors by exciding Mecp2 CDS and exchanging with a GFP cassette. The CBA promoter was removed from the CBA-V5-Mecp2 vector to be replaced by the mouse Mecp2 promoter region (1.4 kb) including the 5′-UTR to generate the M2-Mecp2 vector. Alternatively, the 3′-UTR of the CBA-V5-Mecp2 construct was replaced by an assembled 3′-UTR (aUTR, 223 bp, including portions of the 8.6 kb murine Mecp2 endogenous 3′-UTR, such as: miRNA22-3p, 19-3p and 132-3p target sequence and the distal polyA signal of Mecp2 gene; Gadalla, K. K. E. et al. (2017) Mol. Ther.— Methods Clin. Dev. 5: 180-190) created by gene synthesis (Genewiz) or removed to generate, respectively, the M2c and M2e constructs (FIG. 1a), whereas the M2d construct was made from the CBA-V5-Mecp2 construct by insertion of the murine Mecp2 5′UTR upstream of the V5 sequence (FIG. 1a). In order to generate the (human) iMECP2 vector the murine Mecp2 CDS and 3′-UTR of CBA-VS-Mecp2 construct was replaced by the human MECP2 isoform_2 (NM 001110792.2) CDS including the human 3′-UTR (225 bp) of MECP2 gene. This isoform was chosen since it is the human orthologue of the murine Mecp2 isoform_1. Both NGFR (Nerve Growth Factor Receptor) and Mecp2 (comprehensive of 3′-UTR) amplicons were digested and cloned into a lentiviral vector (LV-Ef1a-GFP) in which the GFP cassette was removed.

The sequence of the CBA-iMECP2 cassette was:

(SEQ ID NO: 13)
cgttacataacttacggtaaatggcccgcctggctgaccgcccaacgac
ccccgcccattgacgtcaataatgacgtatgttcccatagtaacgccaa
tagggactttccattgacgtcaatgggtggagtatttacggtaaactgc
ccacttggcagtacatcaagtgtatcatatgccaagtacgccccctatt
gacgtcaatgacggtaaatggcccgcctggcattatgcccagtacatga
ccttatgggactttcctacttggcagtacatctacgtattagtcatcgc
tattaccatggtcgaggtgagccccacgttctgcttcactctccccatc
tcccccccctccccacccccaattttgtatttatttattttttaattat
tttgtgcagcgatgggggcggggggggggggggggcgcgcgccaggcgg
ggcggggcggggcgaggggcggggcggggcgaggcggagaggtgcggcg
gcagccaatcagagcggcgcgctccgaaagtttccttttatggcgaggc
ggcggcggcggcggccctataaaaagcgaagcgcgcggcgggcggggag
tcgctgcgacgctgccttcgccccgtgccccgctccgccgccgcctcgc
gccgcccgccccggctctgactgaccgcgttactcccacaggtgagcgg
gcgggacggcccttctcctccgggctgtaattagcccgtttagtgaacc
gtcagatcgcctggagacgccatccacgctgttttgacctccatagaag
acaccgggaccgatccagcctccgcggattcgaatcccggccgggaacg
gtgcattggaacgcggattccccgtgccaagagtgacgtaagtaccgcc
tatagagtctataggcccacaaaaaatgctttcttcttttaatatactt
ttttgtttatcttatttctaatactttccctaatctctttctttcaggg
caataatgatacaatgtatcatgcctctttgcaccattctaaagaataa
cagtgataatttctgggttaaggcaatagcaatatttctgcatataaat
atttctgcatataaattgtaactgatgtaagaggtttcatattgctaat
agcagctacaatccagctaccattctgcttttattttatggttgggata
aggctggattattctgagtccaagctaggcccttttgctaatcatgttc
atacctcttatcttcctcccacagctcctgggcaacgtgctggtctgtg
tgctggcccatcactttggcaaagaattgggattcgaacaccggtCGAC
GAATTCGTTAACgGATCCGAACGccaccATGGGCAAGCCTATCCCTAAC
CCTCTGCTGGGCCTGGACTCCACAGGcagCGGCACCGGTatggccgccg
ctgccgccaccgccgccgccgccgccgcgccgagcggaggaggaggagg
aggcgaggaggagagactggaggaaaagtcagaagaccaggatctccag
ggcctcagagacaagccactgaagtttaagaaggcgaagaaagacaaga
aggaggacaaagaaggcaagcatgagccactacaaccttcagcccacca
ttctgcagagccagcagaggcaggcaaagcagaaacatcagaaagctca
ggctctgccccagcagtgccagaagcctcggcttcccccaaacagcggc
gctccattatccgtgaccggggacctatgtatgatgaccccaccttgcc
tgaaggttggacacgaaagcttaaacaaaggaagtctggccgatctgct
ggaaagtatgatgtatatttgatcaatccccagggaaaagcttttcgct
ctaaagtagaattgattgcatactttgaaaaggtgggagacacctcctt
ggaccctaatgattttgacttcacggtaactgggagagggagcccctcc
aggagagagcagaaaccacctaagaagcccaaatctcccaaagctccag
gaactggcaggggtcggggacgccccaaagggagcggcactgggagacc
aaaggcagcagcatcagaaggtgttcaggtgaaaagggtcctggagaag
agccctgggaaacttgttgtcaagatgcctttccaagcatcgcctgggg
gtaagggtgagggaggtggggctaccacatctgcccaggtcatggtgat
caaacgccctggcagaaagcgaaaagctgaagctgacccccaggccatt
cctaagaaacggggtagaaagcctgggagtgtggtggcagctgctgcag
ctgaggccaaaaagaaagccgtgaaggagtcttccatacggtctgtgca
tgagactgtgctccccatcaagaagcgcaagacccgggagacggtcagc
atcgaggtcaaggaagtggtgaagcccctgctggtgtccacccttggtg
agaaaagcgggaagggactgaagacctgcaagagccctgggcgtaaaag
caaggagagcagccccaaggggcgcagcagcagtgcctcctccccacct
aagaaggagcaccatcatcaccaccatcactcagagtccacaaaggccc
ccatgccactgctcccatccccacccccacctgagcctgagagctctga
ggaccccatcagcccccctgagcctcaggacttgagcagcagcatctgc
aaagaagagaagatgccccgaggaggctcactggaaagcgatggctgcc
ccaaggagccagctaagactcagcctatggtcgccaccactaccacagt
tgcagaaaagtacaaacaccgaggggagggagagcgcaaagacattgtt
tcatcttccatgccaaggccaaacagagaggagcctgtggacagccgga
cgcccgtgaccgagagagttagctgactttacatagagcggattgcaaa
gcaaaccaacaagaataaaggcagctgttgtctcttctccttatgggta
gggctctgacaaagcttcccgattaactgaaataaaaaatatttttttt
tctttcagtaaacttagagtttcgtggcttcggggtgggagtagttgga
gcattgggatgtttttcttaccgacaagcacagtcaggttgaagaccta
accaGATATCTCTAGAGATATCCtcgagagatctacgggtggcatccct
gtgacccctccccagtgcctctcctggccctggaagttgccactccagt
gcccaccagccttgtcctaataaaattaagttgcatcattttgtctgac
taggtgtccttctataatattatggggtggaggggggtggtatggagca
aggggcaagttgggaagacaacctgtagggcctgcggggtctattggga
accaagctggagtgcagtggcacaatcttggctcactgcaatctccgcc
tcctgggttcaagcgattctcctgcctcagcctcccgagttgttgggat
tccaggcatgcatgaccaggctcagctaatttttgtttttttggtagag
acggggtttcaccatattggccaggctggtctccaactcctaatctcag
gtgatctacccaccttggcctcccaaattgctgggattacaggcgtgaa
ccactgctcccttccctgtcctt

Sequences of the vectors include:

AAV-CBA-V5-iMecp2-3′pUTR-hGHpA:

(SEQ ID NO: 17)
CCTGCAGGCAGCTGCGCGCTCGCTCGCTCACTGAGGCCGCCCGGGCAAA
GCCCGGGCGTCGGGCGACCTTTGGTCGCCCGGCCTCAGTGAGCGAGCGA
GCGCGCAGAGAGGGAGTGGCCAACTCCATCACTAGGGGTTCCTGCGGCC
GCAACGCGCTAGTTATTAATAGTAATCAATTACGGGGTCATTAGTTCAT
AGCCCATATATGGAGTTCCGCGTTACATAACTTACGGTAAATGGCCCGC
CTGGCTGACCGCCCAACGACCCCCGCCCATTGACGTCAATAATGACGTA
TGTTCCCATAGTAACGCCAATAGGGACTTTCCATTGACGTCAATGGGTG
GAGTATTTACGGTAAACTGCCCACTTGGCAGTACATCAAGTGTATCATA
TGCCAAGTACGCCCCCTATTGACGTCAATGACGGTAAATGGCCCGCCTG
GCATTATGCCCAGTACATGACCTTATGGGACTTTCCTACTTGGCAGTAC
ATCTACGTATTAGTCATCGCTATTACCATGGTCGAGGTGAGCCCCACGT
TCTGCTTCACTCTCCCCATCTCCCCCCCCTCCCCACCCCCAATTTTGTA
TTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGG
CGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGA
AGCGCGCGGCGGGCGGGGAGTCGCTGCGACGCTGCCTTCGCCCCGTGCC
CCGCTCCGCCGCCGCCTCGCGCCGCCCGCCCCGGCTCTGACTGACCGCG
TTACTCCCACAGGTGAGCGGGCGGGACGGCCCTTCTCCTCCGGGCTGTA
ATTAGCCCGTTTAGTGAACCGTCAGATCGCCTGGAGACGCCATCCACGC
TGTTTTGACCTCCATAGAAGACACCGGGACCGATCCAGCCTCCGCGGAT
TCGAATCCCGGCCGGGAACGGTGCATTGGAACGCGGATTCCCCGTGCCA
AGAGTGACGTAAGTACCGCCTATAGAGTCTATAGGCCCACAAAAAATGC
TTTCTTCTTTTAATATACTTTTTTGTTTATCTTATTTCTAATACTTTCC
CTAATCTCTTTCTTTCAGGGCAATAATGATACAATGTATCATGCCTCTT
TGCACCATTCTAAAGAATAACAGTGATAATTTCTGGGTTAAGGCAATAG
CAATATTTCTGCATATAAATATTTCTGCATATAAATTGTAACTGATGTA
AGAGGTTTCATATTGCTAATAGCAGCTACAATCCAGCTACCATTCTGCT
TTTATTTTATGGTTGGGATAAGGCTGGATTATTCTGAGTCCAAGCTAGG
CCCTTTTGCTAATCATGTTCATACCTCTTATCTTCCTCCCACAGCTCCT
GGGCAACGTGCTGGTCTGTGTGCTGGCCCATCACTTTGGCAAAGAATTG
GGATTCGAACACCGGTCGACGAATTCGTTAACGGATCCGAACGCCACCA
TGGGCAAGCCTATCCCTAACCCTCTGCTGGGCCTGGACTCCACAGGCAG
CGGCACCGGTATGGCCGCCGCTGCCGCCACCGCCGCCGCCGCCGCCGCG
CCGAGCGGAGGAGGAGGAGGAGGCGAGGAGGAGAGACTGGAGGAAAAGT
CAGAAGACCAGGATCTCCAGGGCCTCAGAGACAAGCCACTGAAGTTTAA
GAAGGCGAAGAAAGACAAGAAGGAGGACAAAGAAGGCAAGCATGAGCCA
CTACAACCTTCAGCCCACCATTCTGCAGAGCCAGCAGAGGCAGGCAAAG
CAGAAACATCAGAAAGCTCAGGCTCTGCCCCAGCAGTGCCAGAAGCCTC
GGCTTCCCCCAAACAGCGGCGCTCCATTATCCGTGACCGGGGACCTATG
TATGATGACCCCACCTTGCCTGAAGGTTGGACACGAAAGCTTAAACAAA
GGAAGTCTGGCCGATCTGCTGGAAAGTATGATGTATATTTGATCAATCC
CCAGGGAAAAGCTTTTCGCTCTAAAGTAGAATTGATTGCATACTTTGAA
AAGGTGGGAGACACCTCCTTGGACCCTAATGATTTTGACTTCACGGTAA
CTGGGAGAGGGAGCCCCTCCAGGAGAGAGCAGAAACCACCTAAGAAGCC
CAAATCTCCCAAAGCTCCAGGAACTGGCAGGGGTCGGGGACGCCCCAAA
GGGAGCGGCACTGGGAGACCAAAGGCAGCAGCATCAGAAGGTGTTCAGG
TGAAAAGGGTCCTGGAGAAGAGCCCTGGGAAACTTGTTGTCAAGATGCC
TTTCCAAGCATCGCCTGGGGGTAAGGGTGAGGGAGGTGGGGCTACCACA
TCTGCCCAGGTCATGGTGATCAAACGCCCTGGCAGAAAGCGAAAAGCTG
AAGCTGACCCCCAGGCCATTCCTAAGAAACGGGGTAGAAAGCCTGGGAG
TGTGGTGGCAGCTGCTGCAGCTGAGGCCAAAAAGAAAGCCGTGAAGGAG
TCTTCCATACGGTCTGTGCATGAGACTGTGCTCCCCATCAAGAAGCGCA
AGACCCGGGAGACGGTCAGCATCGAGGTCAAGGAAGTGGTGAAGCCCCT
GCTGGTGTCCACCCTTGGTGAGAAAAGCGGGAAGGGACTGAAGACCTGC
AAGAGCCCTGGGCGTAAAAGCAAGGAGAGCAGCCCCAAGGGGCGCAGCA
GCAGTGCCTCCTCCCCACCTAAGAAGGAGCACCATCATCACCACCATCA
CTCAGAGTCCACAAAGGCCCCCATGCCACTGCTCCCATCCCCACCCCCA
CCTGAGCCTGAGAGCTCTGAGGACCCCATCAGCCCCCCTGAGCCTCAGG
ACTTGAGCAGCAGCATCTGCAAAGAAGAGAAGATGCCCCGAGGAGGCTC
ACTGGAAAGCGATGGCTGCCCCAAGGAGCCAGCTAAGACTCAGCCTATG
GTCGCCACCACTACCACAGTTGCAGAAAAGTACAAACACCGAGGGGAGG
GAGAGCGCAAAGACATTGTTTCATCTTCCATGCCAAGGCCAAACAGAGA
GGAGCCTGTGGACAGCCGGACGCCCGTGACCGAGAGAGTTAGCTGACTT
TACATAGAGCGGATTGCAAAGCAAACCAACAAGAATAAAGGCAGCTGTT
GTCTCTTCTCCTTATGGGTAGGGCTCTGACAAAGCTTCCCGATTAACTG
AAATAAAAAATATTTTTTTTTCTTTCAGTAAACTTAGAGTTTCGTGGCT
TCGGGGTGGGAGTAGTTGGAGCATTGGGATGTTTTTCTTACCGACAAGC
ACAGTCAGGTTGAAGACCTAACCAGATATCTCTAGAGATATCCTCGAGA
GATCTACGGGTGGCATCCCTGTGACCCCTCCCCAGTGCCTCTCCTGGCC
CTGGAAGTTGCCACTCCAGTGCCCACCAGCCTTGTCCTAATAAAATTAA
GTTGCATCATTTTGTCTGACTAGGTGTCCTTCTATAATATTATGGGGTG
GAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTT
GGCTCACTGCAATCTCCGCCTCCTGGGTTCAAGCGATTCTCCTGCCTCA
GCCTCCCGAGTTGTTGGGATTCCAGGCATGCATGACCAGGCTCAGCTAA
TTTTTGTTTTTTTGGTAGAGACGGGGTTTCACCATATTGGCCAGGCTGG
TCTCCAACTCCTAATCTCAGGTGATCTACCCACCTTGGCCTCCCAAATT
GCTGGGATTACAGGCGTGAACCACTGCTCCCTTCCCTGTCCTTCTGATT
TTGTAGGTAACCACGTGCGGACCGAGCGGCCGCAGGAACCCCTAGTGAT
GGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGA
GCGAGCGAGCGCGCAGCTGCCTGCAGG

AAV-Mecp2 promoter (1.4 kb)-V5-iMecp2-3′pUTR-hGHpA:

(SEQ ID NO: 18)
CCTGCAGGCAGCTGCGCGCTCGCTCGCTCACTGAGGCCGCCCGGGCAAA
GCCCGGGCGTCGGGCGACCTTTGGTCGCCCGGCCTCAGTGAGCGAGCGA
GCGCGCAGAGAGGGAGTGGCCAACTCCATCACTAGGGGTTCCTGCGGCC
TCTAGTATCGATACTCGAGATTTCAACTGCTACTGTCCTGGTTAAAGCC
TTCATCATCTATCTTTCTTCAACTGCTGCCAGGACCTCTGGACCAGCCA
GTTCTTCATTCTTCACTGGCAACATAGGTTTTATGGTGACAGCTAGTGA
CTCAAATATTTATCAAGGGCTTCTCATCTCAAAATAATCTCCTAGTTCT
TTTGGTGGCCTAGGTCTCTCTCCAGTCACACTGGCCTCCTTAGTAAGGC
AGGCATAGTCCTTCCTTAGAGTGTTTAAACTTGCCTAGAATGTTTTCCC
CAATTACCCATATTGGGAGACGACATGAGGGCAAAAGCTAGAGGGTATC
ATAATAGCACTTCTTTTGTCCTTGCCCTATCTATTTCAAAGTCTTTATC
TCTGTGCAAAATTTTAAGTTCTACTTTCTTGTATGTTTAGTATGACTCT
TCCTTACCAGGAGTCTAGTTTGTCTCCTTGTTCAGTACTAAAACAGTGC
CTAGCAAATAAATGAATAGAGAGGGGAGCCAAATTTGAATCAGAAAGTC
TCTTGTTGCATAGTGTTTAAAAAACAAACAAAGAAAGAAAGTCTCTTGT
TGAGCATTTGTTTAGCACAAAGAGCATTGGATGCTGACTGGTATCAGGG
TAAGGCTGCTTTGACAATGCTCCCTCTGGCCTCACTCCCTTTTATACGT
ACTTCCATCAAACCATCTGATTCAACAATGACAGACCGATCTCTTATGG
GCTTGGCACACACCATCTGCCCATTATAAACGTCTGCAAAGACCAAGGT
TTGATATGTTGATTTTACTGTCAGCCTTAAGAGTGCGACATCTGCTAAT
TTAGTGTAATAATACAATCAGTAGACCCTTTAAAACAAGTCCCTTGGCT
TGGAACAACGCCAGGCTCCTCAACAGGCAACTTTGCTACTTCTACAGAA
AATGATAATAAAGAAATGCTGGTGAAGTCAAATGCTTATCACAATGGTG
AACTACTCAGCAGGGAGGCTCTAATAGGCGCCAAGAGCCTAGACTTCCT
TAAGCGCCAGAGTCCACAAGGGCCCAGTTAATCCTCAACATTCAAATGC
TGCCCACAAAACCAGCCCCTCTGTGCCCTAGCCGCCTCTTTTTTCCAAG
TGACAGTAGAACTCCACCAATCCGCAGCTGAATGGGGTCCGCCTCTTTT
CCCTGCCTAAACAGACAGGAACTCCTGCCAATTGAGGGCGTCACCGCTA
AGGCTCCGCCCCAGCCTGGGCTCCACAACCAATGAAGGGTAATCTCGAC
AAAGAGCAAGGGGTGGGGCGCGGGCGCGCAGGTGCAGCAGCACACAGGC
TGGTCGGGAGGGCGGGGCGCGACGTCTGCCGTGCGGGGTCCCGGCATCG
GTTGCGCGCGCGCTCCCTCCTCTCGGAGAGAGGGCTGTGGTAAAACCCG
TCAATCGCTAGCGGATCCGTTAACGCCACCATGGGCAAGCCTATCCCTA
ACCCTCTGCTGGGCCTGGACTCCACAGGCAGCGGCACCGGTATGGCCGC
CGCTGCCGCCACCGCCGCCGCCGCCGCCGCGCCGAGCGGAGGAGGAGGA
GGAGGCGAGGAGGAGAGACTGGAGGAAAAGTCAGAAGACCAGGATCTCC
AGGGCCTCAGAGACAAGCCACTGAAGTTTAAGAAGGCGAAGAAAGACAA
GAAGGAGGACAAAGAAGGCAAGCATGAGCCACTACAACCTTCAGCCCAC
CATTCTGCAGAGCCAGCAGAGGCAGGCAAAGCAGAAACATCAGAAAGCT
CAGGCTCTGCCCCAGCAGTGCCAGAAGCCTCGGCTTCCCCCAAACAGCG
GCGCTCCATTATCCGTGACCGGGGACCTATGTATGATGACCCCACCTTG
CCTGAAGGTTGGACACGAAAGCTTAAACAAAGGAAGTCTGGCCGATCTG
CTGGAAAGTATGATGTATATTTGATCAATCCCCAGGGAAAAGCTTTTCG
CTCTAAAGTAGAATTGATTGCATACTTTGAAAAGGTGGGAGACACCTCC
TTGGACCCTAATGATTTTGACTTCACGGTAACTGGGAGAGGGAGCCCCT
CCAGGAGAGAGCAGAAACCACCTAAGAAGCCCAAATCTCCCAAAGCTCC
AGGAACTGGCAGGGGTCGGGGACGCCCCAAAGGGAGCGGCACTGGGAGA
CCAAAGGCAGCAGCATCAGAAGGTGTTCAGGTGAAAAGGGTCCTGGAGA
AGAGCCCTGGGAAACTTGTTGTCAAGATGCCTTTCCAAGCATCGCCTGG
GGGTAAGGGTGAGGGAGGTGGGGCTACCACATCTGCCCAGGTCATGGTG
ATCAAACGCCCTGGCAGAAAGCGAAAAGCTGAAGCTGACCCCCAGGCCA
TTCCTAAGAAACGGGGTAGAAAGCCTGGGAGTGTGGTGGCAGCTGCTGC
AGCTGAGGCCAAAAAGAAAGCCGTGAAGGAGTCTTCCATACGGTCTGTG
CATGAGACTGTGCTCCCCATCAAGAAGCGCAAGACCCGGGAGACGGTCA
GCATCGAGGTCAAGGAAGTGGTGAAGCCCCTGCTGGTGTCCACCCTTGG
TGAGAAAAGCGGGAAGGGACTGAAGACCTGCAAGAGCCCTGGGCGTAAA
AGCAAGGAGAGCAGCCCCAAGGGGCGCAGCAGCAGTGCCTCCTCCCCAC
CTAAGAAGGAGCACCATCATCACCACCATCACTCAGAGTCCACAAAGGC
CCCCATGCCACTGCTCCCATCCCCACCCCCACCTGAGCCTGAGAGCTCT
GAGGACCCCATCAGCCCCCCTGAGCCTCAGGACTTGAGCAGCAGCATCT
GCAAAGAAGAGAAGATGCCCCGAGGAGGCTCACTGGAAAGCGATGGCTG
CCCCAAGGAGCCAGCTAAGACTCAGCCTATGGTCGCCACCACTACCACA
GTTGCAGAAAAGTACAAACACCGAGGGGAGGGAGAGCGCAAAGACATTG
TTTCATCTTCCATGCCAAGGCCAAACAGAGAGGAGCCTGTGGACAGCCG
GACGCCCGTGACCGAGAGAGTTAGCTGACTTTACATAGAGCGGATTGCA
AAGCAAACCAACAAGAATAAAGGCAGCTGTTGTCTCTTCTCCTTATGGG
TAGGGCTCTGACAAAGCTTCCCGATTAACTGAAATAAAAAATATTTTTT
TTTCTTTCAGTAAACTTAGAGTTTCGTGGCTTCGGGGTGGGAGTAGTTG
GAGCATTGGGATGTTTTTCTTACCGACAAGCACAGTCAGGTTGAAGACC
TAACCAGATATCTCTAGAGTCGACGAATTCAATAAAAGATCTTTATTTT
CATTAGATCTGTGTGTTGGTTTTTTGTGTGCGGCCGCAGGAACCCCTAG
TGATGGAGTTGGCCACTCCCTCTCTGCGCGCTCGCTCGCTCACTGAGGC
CGGGCGACCAAAGGTCGCCCGACGCCCGGGCTTTGCCCGGGCGGCCTCA
GTGAGCGAGCGAGCGCGCAGCTGCCTGCAGG

AAV-CBA-V5-iMecp2-3′aUTR-hGHpA (M2c):

(SEQ ID NO: 19)
CCTGCAGGCAGCTGCGCGCTCGCTCGCTCACTGAGGCCGCCCGGGCAAA
GCCCGGGCGTCGGGCGACCTTTGGTCGCCCGGCCTCAGTGAGCGAGCGA
GCGCGCAGAGAGGGAGTGGCCAACTCCATCACTAGGGGTTCCTGCGGCC
GCAACGCGCTAGTTATTAATAGTAATCAATTACGGGGTCATTAGTTCAT
AGCCCATATATGGAGTTCCGCGTTACATAACTTACGGTAAATGGCCCGC
CTGGCTGACCGCCCAACGACCCCCGCCCATTGACGTCAATAATGACGTA
TGTTCCCATAGTAACGCCAATAGGGACTTTCCATTGACGTCAATGGGTG
GAGTATTTACGGTAAACTGCCCACTTGGCAGTACATCAAGTGTATCATA
TGCCAAGTACGCCCCCTATTGACGTCAATGACGGTAAATGGCCCGCCTG
GCATTATGCCCAGTACATGACCTTATGGGACTTTCCTACTTGGCAGTAC
ATCTACGTATTAGTCATCGCTATTACCATGGTCGAGGTGAGCCCCACGT
TCTGCTTCACTCTCCCCATCTCCCCCCCCTCCCCACCCCCAATTTTGTA
TTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGG
CGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGA
AGCGCGCGGCGGGCGGGGAGTCGCTGCGACGCTGCCTTCGCCCCGTGCC
CCGCTCCGCCGCCGCCTCGCGCCGCCCGCCCCGGCTCTGACTGACCGCG
TTACTCCCACAGGTGAGCGGGCGGGACGGCCCTTCTCCTCCGGGCTGTA
ATTAGCCCGTTTAGTGAACCGTCAGATCGCCTGGAGACGCCATCCACGC
TGTTTTGACCTCCATAGAAGACACCGGGACCGATCCAGCCTCCGCGGAT
TCGAATCCCGGCCGGGAACGGTGCATTGGAACGCGGATTCCCCGTGCCA
AGAGTGACGTAAGTACCGCCTATAGAGTCTATAGGCCCACAAAAAATGC
TTTCTTCTTTTAATATACTTTTTTGTTTATCTTATTTCTAATACTTTCC
CTAATCTCTTTCTTTCAGGGCAATAATGATACAATGTATCATGCCTCTT
TGCACCATTCTAAAGAATAACAGTGATAATTTCTGGGTTAAGGCAATAG
CAATATTTCTGCATATAAATATTTCTGCATATAAATTGTAACTGATGTA
AGAGGTTTCATATTGCTAATAGCAGCTACAATCCAGCTACCATTCTGCT
TTTATTTTATGGTTGGGATAAGGCTGGATTATTCTGAGTCCAAGCTAGG
CCCTTTTGCTAATCATGTTCATACCTCTTATCTTCCTCCCACAGCTCCT
GGGCAACGTGCTGGTCTGTGTGCTGGCCCATCACTTTGGCAAAGAATTG
GGATTCGAACACCGGTCGACGAATTCGTTAACGGATCCGAACGCCACCA
TGGGCAAGCCTATCCCTAACCCTCTGCTGGGCCTGGACTCCACAGGCAG
CGGCACCGGTATGGCCGCCGCTGCCGCCACCGCCGCCGCCGCCGCCGCG
CCGAGCGGAGGAGGAGGAGGAGGCGAGGAGGAGAGACTGGAGGAAAAGT
CAGAAGACCAGGATCTCCAGGGCCTCAGAGACAAGCCACTGAAGTTTAA
GAAGGCGAAGAAAGACAAGAAGGAGGACAAAGAAGGCAAGCATGAGCCA
CTACAACCTTCAGCCCACCATTCTGCAGAGCCAGCAGAGGCAGGCAAAG
CAGAAACATCAGAAAGCTCAGGCTCTGCCCCAGCAGTGCCAGAAGCCTC
GGCTTCCCCCAAACAGCGGCGCTCCATTATCCGTGACCGGGGACCTATG
TATGATGACCCCACCTTGCCTGAAGGTTGGACACGAAAGCTTAAACAAA
GGAAGTCTGGCCGATCTGCTGGAAAGTATGATGTATATTTGATCAATCC
CCAGGGAAAAGCTTTTCGCTCTAAAGTAGAATTGATTGCATACTTTGAA
AAGGTGGGAGACACCTCCTTGGACCCTAATGATTTTGACTTCACGGTAA
CTGGGAGAGGGAGCCCCTCCAGGAGAGAGCAGAAACCACCTAAGAAGCC
CAAATCTCCCAAAGCTCCAGGAACTGGCAGGGGTCGGGGACGCCCCAAA
GGGAGCGGCACTGGGAGACCAAAGGCAGCAGCATCAGAAGGTGTTCAGG
TGAAAAGGGTCCTGGAGAAGAGCCCTGGGAAACTTGTTGTCAAGATGCC
TTTCCAAGCATCGCCTGGGGGTAAGGGTGAGGGAGGTGGGGCTACCACA
TCTGCCCAGGTCATGGTGATCAAACGCCCTGGCAGAAAGCGAAAAGCTG
AAGCTGACCCCCAGGCCATTCCTAAGAAACGGGGTAGAAAGCCTGGGAG
TGTGGTGGCAGCTGCTGCAGCTGAGGCCAAAAAGAAAGCCGTGAAGGAG
TCTTCCATACGGTCTGTGCATGAGACTGTGCTCCCCATCAAGAAGCGCA
AGACCCGGGAGACGGTCAGCATCGAGGTCAAGGAAGTGGTGAAGCCCCT
GCTGGTGTCCACCCTTGGTGAGAAAAGCGGGAAGGGACTGAAGACCTGC
AAGAGCCCTGGGCGTAAAAGCAAGGAGAGCAGCCCCAAGGGGCGCAGCA
GCAGTGCCTCCTCCCCACCTAAGAAGGAGCACCATCATCACCACCATCA
CTCAGAGTCCACAAAGGCCCCCATGCCACTGCTCCCATCCCCACCCCCA
CCTGAGCCTGAGAGCTCTGAGGACCCCATCAGCCCCCCTGAGCCTCAGG
ACTTGAGCAGCAGCATCTGCAAAGAAGAGAAGATGCCCCGAGGAGGCTC
ACTGGAAAGCGATGGCTGCCCCAAGGAGCCAGCTAAGACTCAGCCTATG
GTCGCCACCACTACCACAGTTGCAGAAAAGTACAAACACCGAGGGGAGG
GAGAGCGCAAAGACATTGTTTCATCTTCCATGCCAAGGCCAAACAGAGA
GGAGCCTGTGGACAGCCGGACGCCCGTGACCGAGAGAGTTAGCTAATCT
AGAAGCTCGCTGATCAGCCTCACAAGAATAAAGGCAGCTGTTGTCTCTT
CAGAAGTAGCTTTGCACTTTTCTAAACTAGGAATATCACCAGGACTGTT
ACTCAATGTGTGCTGCAGGAAAGCACTGATATATTTAAAAACAAAAGGT
GTAACCTATTTATTATATAAAGAGTTTGCCTTATAAATTTACATAAAAA
TGTCCGTTTGTGTCTTTTGTTGTAAAAATCCTCGAGAGATCTACGGGTG
GCATCCCTGTGACCCCTCCCCAGTGCCTCTCCTGGCCCTGGAAGTTGCC
ACTCCAGTGCCCACCAGCCTTGTCCTAATAAAATTAAGTTGCATCATTT
TGTCTGACTAGGTGTCCTTCTATAATATTATGGGGTGGAGGGGGGTGGT
ATGGAGCAAGGGGCAAGTTGGGAAGACAACCTGTAGGGCCTGCGGGGTC
TATTGGGAACCAAGCTGGAGTGCAGTGGCACAATCTTGGCTCACTGCAA
TCTCCGCCTCCTGGGTTCAAGCGATTCTCCTGCCTCAGCCTCCCGAGTT
GTTGGGATTCCAGGCATGCATGACCAGGCTCAGCTAATTTTTGTTTTTT
TGGTAGAGACGGGGTTTCACCATATTGGCCAGGCTGGTCTCCAACTCCT
AATCTCAGGTGATCTACCCACCTTGGCCTCCCAAATTGCTGGGATTACA
GGCGTGAACCACTGCTCCCTTCCCTGTCCTTCTGATTTTGTAGGTAACC
ACGTGCGGACCGAGCGGCCGCAGGAACCCCTAGTGATGGAGTTGGCCAC
TCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGC
GCAGCTGCCTGCAGG

AAV-CBA-5-UTR-V5-iMecp2-3′pUTR-hGHpA (M2d):

(SEQ ID NO: 20)
CCTGCAGGCAGCTGCGCGCTCGCTCGCTCACTGAGGCCGCCCGGGCAAA
GCCCGGGCGTCGGGCGACCTTTGGTCGCCCGGCCTCAGTGAGCGAGCGA
GCGCGCAGAGAGGGAGTGGCCAACTCCATCACTAGGGGTTCCTGCGGCC
GCAACGCGCTAGTTATTAATAGTAATCAATTACGGGGTCATTAGTTCAT
AGCCCATATATGGAGTTCCGCGTTACATAACTTACGGTAAATGGCCCGC
CTGGCTGACCGCCCAACGACCCCCGCCCATTGACGTCAATAATGACGTA
TGTTCCCATAGTAACGCCAATAGGGACTTTCCATTGACGTCAATGGGTG
GAGTATTTACGGTAAACTGCCCACTTGGCAGTACATCAAGTGTATCATA
TGCCAAGTACGCCCCCTATTGACGTCAATGACGGTAAATGGCCCGCCTG
GCATTATGCCCAGTACATGACCTTATGGGACTTTCCTACTTGGCAGTAC
ATCTACGTATTAGTCATCGCTATTACCATGGTCGAGGTGAGCCCCACGT
TCTGCTTCACTCTCCCCATCTCCCCCCCCTCCCCACCCCCAATTTTGTA
TTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGG
CGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGA
AGCGCGCGGCGGGCGGGGAGTCGCTGCGACGCTGCCTTCGCCCCGTGCC
CCGCTCCGCCGCCGCCTCGCGCCGCCCGCCCCGGCTCTGACTGACCGCG
TTACTCCCACAGGTGAGCGGGCGGGACGGCCCTTCTCCTCCGGGCTGTA
ATTAGCCCGTTTAGTGAACCGTCAGATCGCCTGGAGACGCCATCCACGC
TGTTTTGACCTCCATAGAAGACACCGGGACCGATCCAGCCTCCGCGGAT
TCGAATCCCGGCCGGGAACGGTGCATTGGAACGCGGATTCCCCGTGCCA
AGAGTGACGTAAGTACCGCCTATAGAGTCTATAGGCCCACAAAAAATGC
TTTCTTCTTTTAATATACTTTTTTGTTTATCTTATTTCTAATACTTTCC
CTAATCTCTTTCTTTCAGGGCAATAATGATACAATGTATCATGCCTCTT
TGCACCATTCTAAAGAATAACAGTGATAATTTCTGGGTTAAGGCAATAG
CAATATTTCTGCATATAAATATTTCTGCATATAAATTGTAACTGATGTA
AGAGGTTTCATATTGCTAATAGCAGCTACAATCCAGCTACCATTCTGCT
TTTATTTTATGGTTGGGATAAGGCTGGATTATTCTGAGTCCAAGCTAGG
CCCTTTTGCTAATCATGTTCATACCTCTTATCTTCCTCCCACAGCTCCT
GGGCAACGTGCTGGTCTGTGTGCTGGCCCATCACTTTGGCAAAGAATTG
GGATTCGAACACCGGTCGACGTCGGGAGGGCGGGGCGCGACGTCTGCCG
TGCGGGGTCCCGGCATCGGTTGCGCGCGCGCTCCCTCCTCTCGGAGAGA
GGGCTGTGGTAAAACCCGTCCGGAAAGCTAGCGGATCCGAACGCCACCA
TGGGCAAGCCTATCCCTAACCCTCTGCTGGGCCTGGACTCCACAGGCAG
CGGCACCGGTATGGCCGCCGCTGCCGCCACCGCCGCCGCCGCCGCCGCG
CCGAGCGGAGGAGGAGGAGGAGGCGAGGAGGAGAGACTGGAGGAAAAGT
CAGAAGACCAGGATCTCCAGGGCCTCAGAGACAAGCCACTGAAGTTTAA
GAAGGCGAAGAAAGACAAGAAGGAGGACAAAGAAGGCAAGCATGAGCCA
CTACAACCTTCAGCCCACCATTCTGCAGAGCCAGCAGAGGCAGGCAAAG
CAGAAACATCAGAAAGCTCAGGCTCTGCCCCAGCAGTGCCAGAAGCCTC
GGCTTCCCCCAAACAGCGGCGCTCCATTATCCGTGACCGGGGACCTATG
TATGATGACCCCACCTTGCCTGAAGGTTGGACACGAAAGCTTAAACAAA
GGAAGTCTGGCCGATCTGCTGGAAAGTATGATGTATATTTGATCAATCC
CCAGGGAAAAGCTTTTCGCTCTAAAGTAGAATTGATTGCATACTTTGAA
AAGGTGGGAGACACCTCCTTGGACCCTAATGATTTTGACTTCACGGTAA
CTGGGAGAGGGAGCCCCTCCAGGAGAGAGCAGAAACCACCTAAGAAGCC
CAAATCTCCCAAAGCTCCAGGAACTGGCAGGGGTCGGGGACGCCCCAAA
GGGAGCGGCACTGGGAGACCAAAGGCAGCAGCATCAGAAGGTGTTCAGG
TGAAAAGGGTCCTGGAGAAGAGCCCTGGGAAACTTGTTGTCAAGATGCC
TTTCCAAGCATCGCCTGGGGGTAAGGGTGAGGGAGGTGGGGCTACCACA
TCTGCCCAGGTCATGGTGATCAAACGCCCTGGCAGAAAGCGAAAAGCTG
AAGCTGACCCCCAGGCCATTCCTAAGAAACGGGGTAGAAAGCCTGGGAG
TGTGGTGGCAGCTGCTGCAGCTGAGGCCAAAAAGAAAGCCGTGAAGGAG
TCTTCCATACGGTCTGTGCATGAGACTGTGCTCCCCATCAAGAAGCGCA
AGACCCGGGAGACGGTCAGCATCGAGGTCAAGGAAGTGGTGAAGCCCCT
GCTGGTGTCCACCCTTGGTGAGAAAAGCGGGAAGGGACTGAAGACCTGC
AAGAGCCCTGGGCGTAAAAGCAAGGAGAGCAGCCCCAAGGGGCGCAGCA
GCAGTGCCTCCTCCCCACCTAAGAAGGAGCACCATCATCACCACCATCA
CTCAGAGTCCACAAAGGCCCCCATGCCACTGCTCCCATCCCCACCCCCA
CCTGAGCCTGAGAGCTCTGAGGACCCCATCAGCCCCCCTGAGCCTCAGG
ACTTGAGCAGCAGCATCTGCAAAGAAGAGAAGATGCCCCGAGGAGGCTC
ACTGGAAAGCGATGGCTGCCCCAAGGAGCCAGCTAAGACTCAGCCTATG
GTCGCCACCACTACCACAGTTGCAGAAAAGTACAAACACCGAGGGGAGG
GAGAGCGCAAAGACATTGTTTCATCTTCCATGCCAAGGCCAAACAGAGA
GGAGCCTGTGGACAGCCGGACGCCCGTGACCGAGAGAGTTAGCTGACTT
TACATAGAGCGGATTGCAAAGCAAACCAACAAGAATAAAGGCAGCTGTT
GTCTCTTCTCCTTATGGGTAGGGCTCTGACAAAGCTTCCCGATTAACTG
AAATAAAAAATATTTTTTTTTCTTTCAGTAAACTTAGAGTTTCGTGGCT
TCGGGGTGGGAGTAGTTGGAGCATTGGGATGTTTTTCTTACCGACAAGC
ACAGTCAGGTTGAAGACCTAACCAGATATCTCTAGAGATATCCTCGAGA
GATCTACGGGTGGCATCCCTGTGACCCCTCCCCAGTGCCTCTCCTGGCC
CTGGAAGTTGCCACTCCAGTGCCCACCAGCCTTGTCCTAATAAAATTAA
GTTGCATCATTTTGTCTGACTAGGTGTCCTTCTATAATATTATGGGGTG
GAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTT
GGCTCACTGCAATCTCCGCCTCCTGGGTTCAAGCGATTCTCCTGCCTCA
GCCTCCCGAGTTGTTGGGATTCCAGGCATGCATGACCAGGCTCAGCTAA
TTTTTGTTTTTTTGGTAGAGACGGGGTTTCACCATATTGGCCAGGCTGG
TCTCCAACTCCTAATCTCAGGTGATCTACCCACCTTGGCCTCCCAAATT
GCTGGGATTACAGGCGTGAACCACTGCTCCCTTCCCTGTCCTTCTGATT
TTGTAGGTAACCACGTGCGGACCGAGCGGCCGCAGGAACCCCTAGTGAT
GGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGA
GCGAGCGAGCGCGCAGCTGCCTGCAGG

AAV-CBA-V5-iMecp2-hGHpA (M2e):

(SEQ ID NO: 21)
CCTGCAGGCAGCTGCGCGCTCGCTCGCTCACTGAGGCCGCCCGGGCAAA
GCCCGGGCGTCGGGCGACCTTTGGTCGCCCGGCCTCAGTGAGCGAGCGA
GCGCGCAGAGAGGGAGTGGCCAACTCCATCACTAGGGGTTCCTGCGGCC
GCAACGCGCTAGTTATTAATAGTAATCAATTACGGGGTCATTAGTTCAT
AGCCCATATATGGAGTTCCGCGTTACATAACTTACGGTAAATGGCCCGC
CTGGCTGACCGCCCAACGACCCCCGCCCATTGACGTCAATAATGACGTA
TGTTCCCATAGTAACGCCAATAGGGACTTTCCATTGACGTCAATGGGTG
GAGTATTTACGGTAAACTGCCCACTTGGCAGTACATCAAGTGTATCATA
TGCCAAGTACGCCCCCTATTGACGTCAATGACGGTAAATGGCCCGCCTG
GCATTATGCCCAGTACATGACCTTATGGGACTTTCCTACTTGGCAGTAC
ATCTACGTATTAGTCATCGCTATTACCATGGTCGAGGTGAGCCCCACGT
TCTGCTTCACTCTCCCCATCTCCCCCCCCTCCCCACCCCCAATTTTGTA
TTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGG
CGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGA
AGCGCGCGGCGGGCGGGGAGTCGCTGCGACGCTGCCTTCGCCCCGTGCC
CCGCTCCGCCGCCGCCTCGCGCCGCCCGCCCCGGCTCTGACTGACCGCG
TTACTCCCACAGGTGAGCGGGCGGGACGGCCCTTCTCCTCCGGGCTGTA
ATTAGCCCGTTTAGTGAACCGTCAGATCGCCTGGAGACGCCATCCACGC
TGTTTTGACCTCCATAGAAGACACCGGGACCGATCCAGCCTCCGCGGAT
TCGAATCCCGGCCGGGAACGGTGCATTGGAACGCGGATTCCCCGTGCCA
AGAGTGACGTAAGTACCGCCTATAGAGTCTATAGGCCCACAAAAAATGC
TTTCTTCTTTTAATATACTTTTTTGTTTATCTTATTTCTAATACTTTCC
CTAATCTCTTTCTTTCAGGGCAATAATGATACAATGTATCATGCCTCTT
TGCACCATTCTAAAGAATAACAGTGATAATTTCTGGGTTAAGGCAATAG
CAATATTTCTGCATATAAATATTTCTGCATATAAATTGTAACTGATGTA
AGAGGTTTCATATTGCTAATAGCAGCTACAATCCAGCTACCATTCTGCT
TTTATTTTATGGTTGGGATAAGGCTGGATTATTCTGAGTCCAAGCTAGG
CCCTTTTGCTAATCATGTTCATACCTCTTATCTTCCTCCCACAGCTCCT
GGGCAACGTGCTGGTCTGTGTGCTGGCCCATCACTTTGGCAAAGAATTG
GGATTCGAACACCGGTCGACGAATTCGTTAACGGATCCGAACGCCACCA
TGGGCAAGCCTATCCCTAACCCTCTGCTGGGCCTGGACTCCACAGGCAG
CGGCACCGGTATGGCCGCCGCTGCCGCCACCGCCGCCGCCGCCGCCGCG
CCGAGCGGAGGAGGAGGAGGAGGCGAGGAGGAGAGACTGGAGGAAAAGT
CAGAAGACCAGGATCTCCAGGGCCTCAGAGACAAGCCACTGAAGTTTAA
GAAGGCGAAGAAAGACAAGAAGGAGGACAAAGAAGGCAAGCATGAGCCA
CTACAACCTTCAGCCCACCATTCTGCAGAGCCAGCAGAGGCAGGCAAAG
CAGAAACATCAGAAAGCTCAGGCTCTGCCCCAGCAGTGCCAGAAGCCTC
GGCTTCCCCCAAACAGCGGCGCTCCATTATCCGTGACCGGGGACCTATG
TATGATGACCCCACCTTGCCTGAAGGTTGGACACGAAAGCTTAAACAAA
GGAAGTCTGGCCGATCTGCTGGAAAGTATGATGTATATTTGATCAATCC
CCAGGGAAAAGCTTTTCGCTCTAAAGTAGAATTGATTGCATACTTTGAA
AAGGTGGGAGACACCTCCTTGGACCCTAATGATTTTGACTTCACGGTAA
CTGGGAGAGGGAGCCCCTCCAGGAGAGAGCAGAAACCACCTAAGAAGCC
CAAATCTCCCAAAGCTCCAGGAACTGGCAGGGGTCGGGGACGCCCCAAA
GGGAGCGGCACTGGGAGACCAAAGGCAGCAGCATCAGAAGGTGTTCAGG
TGAAAAGGGTCCTGGAGAAGAGCCCTGGGAAACTTGTTGTCAAGATGCC
TTTCCAAGCATCGCCTGGGGGTAAGGGTGAGGGAGGTGGGGCTACCACA
TCTGCCCAGGTCATGGTGATCAAACGCCCTGGCAGAAAGCGAAAAGCTG
AAGCTGACCCCCAGGCCATTCCTAAGAAACGGGGTAGAAAGCCTGGGAG
TGTGGTGGCAGCTGCTGCAGCTGAGGCCAAAAAGAAAGCCGTGAAGGAG
TCTTCCATACGGTCTGTGCATGAGACTGTGCTCCCCATCAAGAAGCGCA
AGACCCGGGAGACGGTCAGCATCGAGGTCAAGGAAGTGGTGAAGCCCCT
GCTGGTGTCCACCCTTGGTGAGAAAAGCGGGAAGGGACTGAAGACCTGC
AAGAGCCCTGGGCGTAAAAGCAAGGAGAGCAGCCCCAAGGGGCGCAGCA
GCAGTGCCTCCTCCCCACCTAAGAAGGAGCACCATCATCACCACCATCA
CTCAGAGTCCACAAAGGCCCCCATGCCACTGCTCCCATCCCCACCCCCA
CCTGAGCCTGAGAGCTCTGAGGACCCCATCAGCCCCCCTGAGCCTCAGG
ACTTGAGCAGCAGCATCTGCAAAGAAGAGAAGATGCCCCGAGGAGGCTC
ACTGGAAAGCGATGGCTGCCCCAAGGAGCCAGCTAAGACTCAGCCTATG
GTCGCCACCACTACCACAGTTGCAGAAAAGTACAAACACCGAGGGGAGG
GAGAGCGCAAAGACATTGTTTCATCTTCCATGCCAAGGCCAAACAGAGA
GGAGCCTGTGGACAGCCGGACGCCCGTGACCGAGAGAGTTAGCTGAGTC
GAGAGATCTACGGGTGGCATCCCTGTGACCCCTCCCCAGTGCCTCTCCT
GGCCCTGGAAGTTGCCACTCCAGTGCCCACCAGCCTTGTCCTAATAAAA
TTAAGTTGCATCATTTTGTCTGACTAGGTGTCCTTCTATAATATTATGG
GGTGGAGGGGGGTGGTATGGAGCAAGGGGCAAGTTGGGAAGACAACCTG
TAGGGCCTGCGGGGTCTATTGGGAACCAAGCTGGAGTGCAGTGGCACAA
TCTTGGCTCACTGCAATCTCCGCCTCCTGGGTTCAAGCGATTCTCCTGC
CTCAGCCTCCCGAGTTGTTGGGATTCCAGGCATGCATGACCAGGCTCAG
CTAATTTTTGTTTTTTTGGTAGAGACGGGGTTTCACCATATTGGCCAGG
CTGGTCTCCAACTCCTAATCTCAGGTGATCTACCCACCTTGGCCTCCCA
AATTGCTGGGATTACAGGCGTGAACCACTGCTCCCTTCCCTGTCCTTCT
GATTTTGTAGGTAACCACGTGCGGACCGAGCGGCCGCAGGAACCCCTAG
TGATGGAGTTGGCCACTCCCTCTCTGCGCGCTCGCTCGCTCACTGAGGC
CGGGCGACCAAAGGTCGCCCGACGCCCGGGCTTTGCCCGGGCGGCCTCA
GTGAGCGAGCGAGCGCGCAGCTGCCTGCAGG

### AAV-CBA-iMecp2-3′pUTR-hGHpA (M2f):

(SEQ ID NO: 25)
cctgcaggcagctgcgcgctcgctcgctcactgaggccgcccgggcaaa
gcccgggcgtcgggcgacctttggtcgcccggcctcagtgagcgagcga
gcgcgcagagagggagtggccaactccatcactaggggttcctgcggcc
gcaacgcgctagttattaatagtaatcaattacggggtcattagttcat
agcccatatatggagttccgcgttacataacttacggtaaatggcccgc
ctggctgaccgcccaacgacccccgcccattgacgtcaataatgacgta
tgttcccatagtaacgccaatagggactttccattgacgtcaatgggtg
gagtatttacggtaaactgcccacttggcagtacatcaagtgtatcata
tgccaagtacgccccctattgacgtcaatgacggtaaatggcccgcctg
gcattatgcccagtacatgaccttatgggactttcctacttggcagtac
atctacgtattagtcatcgctattaccatggtcgaggtgagccccacgt
tctgcttcactctccccatctcccccccctccccacccccaattttgta
tttatttattttttaattattttgtgcagcgatgggggcgggggggggg
ggggggcgcgcgccaggcggggcggggcggggcgaggggcggggcgggg
cgaggcggagaggtgcggcggcagccaatcagagcggcgcgctccgaaa
gtttccttttatggcgaggcggcggcggcggcggccctataaaaagcga
agcgcgcggcgggcggggagtcgctgcgacgctgccttcgccccgtgcc
ccgctccgccgccgcctcgcgccgcccgccccggctctgactgaccgcg
ttactcccacaggtgagcgggcgggacggcccttctcctccgggctgta
attagcccgtttagtgaaccgtcagatcgcctggagacgccatccacgc
tgttttgacctccatagaagacaccgggaccgatccagcctccgcggat
tcgaatcccggccgggaacggtgcattggaacgcggattccccgtgcca
agagtgacgtaagtaccgcctatagagtctataggcccacaaaaaatgc
tttcttcttttaatatacttttttgtttatcttatttctaatactttcc
ctaatctctttctttcagggcaataatgatacaatgtatcatgcctctt
tgcaccattctaaagaataacagtgataatttctgggttaaggcaatag
caatatttctgcatataaatatttctgcatataaattgtaactgatgta
agaggtttcatattgctaatagcagctacaatccagctaccattctgct
tttattttatggttgggataaggctggattattctgagtccaagctagg
cccttttgctaatcatgttcatacctcttatcttcctcccacagctcct
gggcaacgtgctggtctgtgtgctggcccatcactttggcaaagaattg
ggattcgaacaccggtCGACGAATTCGTTAACGGATCCgccaccatggc
cgccgctgccgccaccgccgccgccgccgccgcgccgagcggaggagga
ggaggaggcgaggaggagagactggaggaaaagtcagaagaccaggatc
tccagggcctcagagacaagccactgaagtttaagaaggcgaagaaaga
caagaaggaggacaaagaaggcaagcatgagccactacaaccttcagcc
caccattctgcagagccagcagaggcaggcaaagcagaaacatcagaaa
gctcaggctctgccccagcagtgccagaagcctcggcttcccccaaaca
gcggcgctccattatccgtgaccggggacctatgtatgatgaccccacc
ttgcctgaaggttggacacgaaagcttaaacaaaggaagtctggccgat
ctgctggaaagtatgatgtatatttgatcaatccccagggaaaagcttt
tcgctctaaagtagaattgattgcatactttgaaaaggtgggagacacc
tccttggaccctaatgattttgacttcacggtaactgggagagggagcc
cctccaggagagagcagaaaccacctaagaagcccaaatctcccaaagc
tccaggaactggcaggggtcggggacgccccaaagggagcggcactggg
agaccaaaggcagcagcatcagaaggtgttcaggtgaaaagggtcctgg
agaagagccctgggaaacttgttgtcaagatgcctttccaagcatcgcc
tgggggtaagggtgagggaggtggggctaccacatctgcccaggtcatg
gtgatcaaacgccctggcagaaagcgaaaagctgaagctgacccccagg
ccattcctaagaaacggggtagaaagcctgggagtgtggtggcagctgc
tgcagctgaggccaaaaagaaagccgtgaaggagtcttccatacggtct
gtgcatgagactgtgctccccatcaagaagcgcaagacccgggagacgg
tcagcatcgaggtcaaggaagtggtgaagcccctgctggtgtccaccct
tggtgagaaaagcgggaagggactgaagacctgcaagagccctgggcgt
aaaagcaaggagagcagccccaaggggcgcagcagcagtgcctcctccc
cacctaagaaggagcaccatcatcaccaccatcactcagagtccacaaa
ggcccccatgccactgctcccatccccacccccacctgagcctgagagc
tctgaggaccccatcagcccccctgagcctcaggacttgagcagcagca
tctgcaaagaagagaagatgccccgaggaggctcactggaaagcgatgg
ctgccccaaggagccagctaagactcagcctatggtcgccaccactacc
acagttgcagaaaagtacaaacaccgaggggagggagagcgcaaagaca
ttgtttcatcttccatgccaaggccaaacagagaggagcctgtggacag
ccggacgcccgtgaccgagagagttagctgactttacatagagcggatt
gcaaagcaaaccaacaagaataaaggcagctgttgtctcttctccttat
gggtagggctctgacaaagcttcccgattaactgaaataaaaaatattt
ttttttctttcagtaaacttagagtttcgtggcttcggggtgggagtag
ttggagcattgggatgtttttcttaccgacaagcacagtcaggttgaag
acctaaccaGATATCTCTAGAGATATCCtcgagagatctacgggtggca
tccctgtgacccctccccagtgcctctcctggccctggaagttgccact
ccagtgcccaccagccttgtcctaataaaattaagttgcatcattttgt
ctgactaggtgtccttctataatattatggggtggaggggggtggtatg
gagcaaggggcaagttgggaagacaacctgtagggcctgcggggtctat
tgggaaccaagctggagtgcagtggcacaatcttggctcactgcaatct
ccgcctcctgggttcaagcgattctcctgcctcagcctcccgagttgtt
gggattccaggcatgcatgaccaggctcagctaatttttgtttttttgg
tagagacggggtttcaccatattggccaggctggtctccaactcctaat
ctcaggtgatctacccaccttggcctcccaaattgctgggattacaggc
gtgaaccactgctcccttccctgtccttctgattttgtaggtaaccacg
tgcggaccgagcggccgcaggaacccctagtgatggagttggccactcc
ctctctgcgcgctcgctcgctcactgaggccgggcgaccaaaggtcgcc
cgacgcccgggctttgcccgggcggcctcagtgagcgagcgagcgcgca
gctgcctgcagg

Virus Production and Purification

Lentiviral replication-incompetent, VSVg-coated lentiviral particles were packaged in 293T cells (Vierbuchen, T. et al. (2010) Nature 463: 1035-1041). Cells were transfected with 30 μg of vector and packaging constructs, according to a conventional CaCl2 transfection protocol. After 30 h, medium was collected, filtered through 0.44 μm cellulose acetate and centrifuged at 20000 rpm for 2 h at 20° C. in order to concentrate the virus.

AAV replication-incompetent, recombinant viral particles were produced 293T cells, cultured in Dulbecco Modified Eagle Medium—high glucose (Sigma-Aldrich) containing 10% fetal bovine serum (Sigma-Aldrich), 1% non-essential amino acids (Gibco), 1% sodium pyruvate (Sigma-Aldrich), 1% glutamine (Sigma-Aldrich) and 1% penicillin/streptomycin (Sigma-Aldrich). Cells were split every 3-4 days using Trypsin 0.25% (Sigma-Aldrich). Replication-incompetent, recombinant viral particles were produced in 293T cells by polyethylenimine (PEI) (Polyscience) co-transfection of three different plasmids: transgene-containing plasmid, packaging plasmid for rep and cap genes and pHelper (Agilent) for the three adenoviral helper genes. The cells and supernatant were harvested at 120 hrs. Cells were lysed in hypertonic buffer (40 mM Tris, 500 mM NaCl, 2 mM MgCl₂, pH 8) containing 100 U/ml Salt Active Nuclease (SAN, Arcticzymes) for 1 h at 37° C., whereas the viral particles present in the supernatant were concentrated by precipitation with 8% PEG8000 (Polyethylene glycol 8000, Sigma-Aldrich) and then added to supernatant for an additional incubation of 30 min at 37° C. In order to clarify the lysate cellular debris where separated by centrifugation (4000 g, 30 min). The viral phase was isolated by iodixanol step gradient (15%, 25%, 40%, 60% Optiprep, Sigma-Aldrich) in the 40% fraction and concentrated in PBS (Phosphate Buffer Saline) with 100 K cut-off concentrator (Amicon Ultra15, MERCK-Millipore). Virus titers were determined using AAVpro© Titration Kit Ver2 (TaKaRa).

Primary Mouse Neuronal Cultures

Primary Neuronal culture were prepared at embryonic day 17.5 (E17.5) from male mouse embryos. Briefly, cortices were individually dissected, sequentially incubated in trypsin (0.005%, 20 min at 37° C., Sigma-Aldrich) and DNAse (0.1 mg/mL, 5 min at room temperature, Sigma-Aldrich) in HBSS (Hank's buffered salt solution without Ca²+ and Mg²+, Euroclone). Cells were finally and plated on poly-L-lysine (Sigma-Aldrich) coated dishes (2.0×10⁵ cells/cm²) in Neurobasal medium (TermoFisher Scientific) enriched with 0.6% glucose (Sigma-Aldrich), 0.2% penicillin/streptomycin (Sigma-Aldrich), 0.25% L-glutamine (Sigma-Aldrich) and 1% B27 (TermoFisher Scientific). Virus particles were directly added to cultured neurons 3 days after seeding, with a final concentration 10¹⁰ vg/ml.

AAV-PHP.eB Vector Injection, Mouse Phenotyping and Tissue Collection

Vascular injection was performed in a restrainer that positioned the tail in a heated groove. The tail was swabbed with alcohol and then injected intravenously with a variable viral concentration (from 1×10¹⁰ to 1×10¹³ vg/mL) depending on the experimental setup in a total volume of 100 μl of AAV-PHP.eB particles in PBS.

Juvenile WT and Mecp2^{-/y} mice were randomised in groups and injected in the tail vein between 25 and 30 days of age. Adult WT were injected in a similar time window whereas Mecp2^{+/-} females were treated intravenously after five months of life. Following injection, all mice were weighed twice a week. Phenotyping was carried out, blind to genotype and treatment, twice a week. Mice symptoms were scored on an aggregate severity scale (0=absent; 1=present; 2=severe) comprising mobility, gait, breathing, hindlimb clasping, tremor, and general condition. The balance and the motor coordination were assessed by the Beam Balance test. Briefly mice were placed on the tip of the beam at the “start-point” facing towards the beam. The number of foot-slips (error numbers) and total time on beam (crossing time) from “start” to “end” points were noted. If a mouse fell, the animal was returned to the site where it fell from, until completion of beam crossing.

The Open Field test was performed in a rectangular arena of 36×24 cm. Mice were testing in a 10 minutes session measured as an index of anxiety and horizontal exploratory activity in a novel environment was assessed.

For serum analysis blood samples were collected from living animals using retro-orbital bleeding procedure with non-heparinised capillaries. Upon blood clothing cell fraction was pelleted (5 min, 13000 rpm) and supernatant recovered. When the body loss reached 20% of total weight mice were sacrificed and tissues harvested. Briefly, mice were anesthetised with ketamine/xylazine and transcardially perfused with 0.1 M phosphate buffer (PB) at room temperature (RT) at pH 7.4. Upon this treatment brain, liver and spleen were collected. Brain hemispheres were separated: one half was post-fixed in 4% PFA for two days and then soaked in cryoprotective solution (30% sucrose in PBS) for immunofluorescence analysis the other further sectioned in different areas (cortex, striatum, cerebellum) quick frozen on dry-ice for Western blot, RNA and DNA extraction. Liver specimens were collected similarly. Spleens were collected in PBS for subsequent splenocyte extraction.

Total RNA-DNA Isolation and qRT-PCR for Mecp2 RNA Stability, Biodistribution and Gene Expression

Total RNA was isolated from primary neurons and animal tissues (cortex and liver) using the Qiagen RNeasy mini kit (QIAGEN). About 1 μg of RNA was reverse transcribed with random hexamers as primers using ImProm-II™ Reverse Transcription System (Promega). For quantitative real time PCR (qRT-PCR), Titan HotTaq EvaGreen qPCR mix (BioAtlas) was used and expression levels were normalised with respect to β-actin expression. The results were reported as the fold change (2^{-ΔΔCt}) of viral Mecp2 relative to endogenous Mecp2.

The stability of endogenous and viral Mecp2 RNA was assessed by qRT-PCR. The RNA was isolated at the indicated time-points from neurons (WT uninfected and infected with iMecp2 vector and Mecp2^{-/y} infected with iMecp2 vector) treated with 10 mg/mL of Actinomycin D (Sigma-Aldrich).

Total DNA was isolated from primary neurons and animal tissues (cortex and liver) using the Qiagen DNeasy Blood & Tissue Kits (QIAGEN). The quantification of vector transgene expression was calculated by qRT-PCR relative to the endogenous Mecp2. The DNA levels were normalised against an amplicon from a single-copy mouse gene, Lmnb2, amplified from genomic DNA.

RiboLace

The RiboLace kit (IMMAGINA Biotechnology S.r.l) was used to isolate from primary neurons (wild-type uninfected and infected with iMecp2 vector and Mecp2^{-/y} infected with iMecp2 vector) at DIV 14 two distinct fraction: total RNA and RNA associated to the active ribosomes, according to manufacturer's instructions. Following the isolation, about 100 ng of RNA was reverse transcribed and amplified by qRT-PCR as described above using the oligonucleotide primers to amplify endogenous Mecp2, viral Mecp2 and 18S as housekeeping gene. The result was reported as fold change (2^{-ΔΔCt}) in gene expression of viral Mecp2 relative to endogenous Mecp2, in the captured fraction normalised on total RNA.

Generation of a MECP2-KO Human iPS Cell (iPSC) Line

Control human iPSC cell line were generated from neonatal primary fibroblasts obtained from ATCC. iPSCs were maintained in feeder-free conditions in mTeSR1 (Stem Cell Technologies) and seeded in HESC qualified matrigel (Corning)-coated 6-well plates. To generate the MECP2-KO cell line, an sgRNA (sgMECP2: 5′-aagcttaagcaaaggaaatc-3′) was designed on the third exon of MECP2 using the software crispor.tefor.net. The oligo (Sigma-Aldrich) pairs encoding the 20-nt guide sequences were cloned into the LV-U6-filler-gRNA-EF1α-Blast (Rubio et al., 2016). Wild-type human iPSCs were then co-transfected with the LV-U6-sgMECP2-EF1α-Blast and the pCAG-Cas9-Puro using the Lipofectamine Stem Cells Transfection Reagent (ThermoFisher Scientific) (Giannelli, S. G. et al. (2018) Hum. Mol. Genet. 27: 761-779). Co-transfected colonies were then selected by the combination of puromycin (1 μg/ml, Sigma) and blastidicin (10 μg/ml, ThermoFisher Scientific) and then isolated through single colony picking. Finally, MECP2-KO cell lines were confirmed by Sanger Sequencing and protein absence was further corroborated by immunofluorescence.

Differentiation of Human iPSCs in Cortical Neurons

iPSCs were initially differentiated in Neural Progenitors Cells (NPCs) as described (Iannielli, A. et al. (2018) Cell Rep. 22: 2066-2079). NPCs were, then, dissociated with Accutase (Sigma-Aldrich) and plated on matrigel-coated 6-well plates (3*10⁵ cells per well) in NPC medium. Two days after, the medium was changed with the differentiation medium containing Neurobasal (ThermoFisher Scientific), 1% Pen/Strep (Sigma-Aldrich), 1% Glutamine (Sigma-Aldrich), 1:50 B27 minus vitamin A (ThermoFisher Scientific), 5 μM XAV939 (Sigma-Aldrich), 10 μM SU5402 (Sigma-Aldrich), 8 μM PD0325901 (Tocris Bioscience), and 10 μM DAPT (Sigma-Aldrich) was added and kept for 3 days. After 3 days, the cells were dissociated with Accutase (Sigma-Aldrich) and plated on poly-L-lysine (Sigma-Aldrich)/laminin (Sigma-Aldrich)-coated 12-well plates (2*10⁵ cells per well) and 24-well plates (1*10⁵ cells per well) in maturation medium containing Neurobasal (ThermoFisher Scientific), 1% Pen/Strep (Sigma-Aldrich), 1% Glutamine (Sigma-Aldrich), 1:50 B27 minus vitamin A (ThermoFisher Scientific), 25 ng/ml human BDNF (PeproTech), 20 μM Ascorbic Acid (Sigma-Aldrich), 250 μM Dibutyryl cAMP (Sigma-Aldrich), 10 μM DAPT (Sigma-Aldrich) and Laminin for terminal differentiation. At this stage half of the medium was changed every 2-3 days. Viral particles were directly added to cultured neurons after three weeks of differentiation, with a final concentration 10¹⁰ vg/ml. All the analysis was conducted one week after the infection.

Immunofluorescence

Primary neurons were fixed with ice-cold 4% paraformaldehyde (PFA) for 30 min at 4° C., washed with PBS (3×) and incubated with 10% donkey serum and 3% Triton X-100 for 1 h at RT to saturate the unspecific binding site before the overnight incubation at 4° C. with the primary antibody. Upon wash with PBS (3×), cells were incubated for 1 h at RT in blocking solution with DAPI and with Alexa Fluor-488 and Alexa Fluor-594 anti-rabbit or anti-mouse secondary antibodies. After PBS washes (3×), cells were mounted with fluorescent mounting medium (Dako). Images were captured with a Nikon Eclipse 600 fluorescent microscope.

Tissues were sectioned using cryostat after optimal cutting temperature compound (OCT) embedding in dry ice. Free-floating 50 μm-thick coronal sections were rinsed in PBS and were incubated with 10% donkey serum (Sigma-Aldrich) and 3% Triton X-100 (Sigma-Aldrich) for 1 h at RT to saturate the unspecific binding site before the overnight incubation at 4° C. with the primary antibody (diluted in the blocking solution).

Upon wash with PBS (3×), sections were incubated for 1 h at RT in blocking solution with DAPI (1:1000, Sigma-Aldrich) and with Alexa Fluor-488 and Alexa Fluor-594 anti-rabbit or anti-mouse secondary antibodies (1:1000, ThermoFisher Scientific). After PBS washes (3×), sections were mounted with fluorescent mounting medium (Dako). Confocal images were captured at ×40 or ×63 magnification with Leica TCS SP5 Laser Scanning Confocal microscope (Leica Microsystems Ltd).

Cell and tissue where stained with the following primary antibody: rabbit anti-MeCP2 (1:500; Cell Signaling Technology), mouse anti-V5 (1:500; ThermoFisher Scientific), mouse anti-NeuN (1:300; Merck Millipore), rabbit anti-GFAP (1:500; Dako), rabbit anti-GABA (1:500; Sigma-Aldrich).

Western Blot

Protein extracts were prepared in RIPA buffer (10 mM Tris-HCl pH7.4, 150 mM NaCl, 1 mM EGTA, 0.5% Triton and complete 1% protease and phosphatase inhibitor mixture, Roche Diagnostics). Primary neurons, brain and liver lysate samples (50 μg protein lysates) were separated using 8% polyacrylamide gel and then transferred to PVDF membranes. Membranes were incubated overnight at 4° C. with the following primary antibodies in 1× PBST with 5% w/v nonfat dry: rabbit anti-MeCP2 (1:1000; Sigma), mouse anti-V5 (1:1000; ThermoFisher Scientific), rabbit anti-pS6 235/236 (1:500, Cell Signaling), rabbit anti-S6 (1:500, Cell Signaling), rabbit anti-Calnexin (1:50000, Sigma), mouse anti-13-Actin (1:50000; Sigma) or the mice serum (1:200) extracted through retro-orbital bleeding followed by centrifugation (10 min, RT, 13000 rpm). Subsequently, membranes were incubated with the corresponding horseradish peroxidase (HRP)-conjugated secondary antibodies (1:10000; Dako). The signal was then revealed with a chemiluminescence solution (ECL reagent, RPN2232; GE Healthcare) and detected with the ChemiDoc imaging system (Bio-Rad).

Antibody Detection in Serum

Serum was extract from mice through retro-orbital bleeding followed by centrifugation (10 min, RT, 13000 rpm). In order to test the sera by immunofluorescence we generated a P19 Mecp2^{-/y} cell line using pCAG-spCas9 and sgMecp2. Moreover, isolation of a clone carrying a frameshift mutation (−14 nt) in the exon 3 of Mecp2 gene that ensured ablation of MeCP2 protein (as tested by immunofluorescence). Mecp2^{-/y} P19 cells were transfected with GFP only (negative control) or with GFP and iMecp2, cells were fixed and incubated in blocking solution as describe above before the overnight incubation at 4° C. with the primary antibody mix composed of chicken anti-GFP and either a rabbit anti-MeCP2 (positive control) or the mice serum (1:50) (for details see the above Immunofluorescence paragraph). For Western blot assay, the proteins Mecp2^{-/y} and WT cortices were extracted and separated using 8% polyacrylamide gels. Membranes were incubated overnight at 4° C. with a rabbit anti-MeCP2 (positive control) or the mice serum (1:200) (for details see the Western blot paragraph).

Fluorescent Intensity Measurements

Brain sections were processed for immunolabelling as above and confocal images were captured at ×63 magnification with Leica TCS SP5 Laser Scanning Confocal microscope (Leica Microsystems Ltd) using the identical settings. Then, the quantification of the signal was performed using ImageJ software (NIH, US). The fluorescent signal was measured as described by Iannielli, A. et al. (2018) Cell Reports 22: 2066-2079.

Spleen Cell Isolation

Spleens were triturated in PBS and cell pelleted (7 min, 1500 rpm) to be incubated in ACK buffer (5 min, RT) to lyse blood cells. The reaction was stopped diluting 1:10 the ACK buffer in PBS and removing it by centrifugation (7 min, 1500 rpm). Cells were than counted and frozen in FBS:DMSO (9:1 ratio) solution.

Flow Cytometry

Spleen cells were incubated with 25 μl of Ab mix for 30 min at 4°. Red blood cells lysis was performed with BD Phosflow (BD Bioscience, 558049) according to manufacturer's instruction. Labelled cells were washed two times with PBS 1% FBS and analysed with a BD LSRFortessa analyser, results were analysed with FlowJo 10 software.

T Cell Proliferation

Spleen cells were labelled with Cell Proliferation Dye eFluor® 670 (eBioscience, CA, USA) according to manufacturer's instructions and stimulated with 10⁴ bone-marrow derived DC transduce with lentiviral vector encoding for Mecp2 (10:1, T:DC) in RPMI 1640 medium (Lonza, Switzerland), with 10% FBS (Euroclone, ECS0180L), 100 U/ml penicillin/streptomycin (Lonza, 17-602E), 2 mM L-glutamine (Lonza, 17-605E), Minimum Essential Medium Non-Essential Amino Acids (MEM NEAA) (GIBCO, 11140-035), 1 mM Sodium Pyruvate (GIBCO, 11360-039), 50 nM 2-Mercaptoethanol (GIBCO, 31350-010). Alternatively, spleen cells were stimulated with anti-CD3e monoclonal Ab (BD Bioscience, 553058) (1 μg/mL). After 4 days, T cells were collected, washed, and their phenotype and proliferation were analysed by flow cytometry.

Elispot Assays

CD8⁺ T cells were magnetically isolated from the spleen (Miltenyi Biotec, 130-104-075). 10⁵ CD8⁺ T cells were plated in triplicate in ELISPOT plates (Millipore, Bedford, Mass.) pre-coated with anti-IFN-γ capture monoclonal Ab (2.5 μg/mL; BD Pharmingen, R46A2) in the presence of IL-2 (50 U/mL; BD Pharmingen) and 10⁵ irradiated (6000 rad) un-transduced or LV.Mecp2-transduced autologous EL-4 cells. After 42 hours of incubation at 37° C. 5% CO₂, plates were washed and IFN-γ—producing cells were detected by biotin-conjugated anti-IFN-γ monoclonal Ab (0.5 μg/mL; BD Pharmingen, XMG 1.2). Streptavidin-HRP conjugate (Roche) was added. Total splenocytes or total BM (0.35×10⁶ cells/well) were plated in complete RPMI in triplicate in ELISPOT plates pre-coated with rhIDUA (2 μg/well). After 24 hours of incubation at 37° C. 5% CO₂, plates were washed and anti-IDUA IgG secreting cells were detected with peroxidase-conjugated rabbit anti-mouse immunoglobulin (SIGMA A2554). All plates were reacted with H₂O₂ and 3-Amino-9-ethylcarbazole (SIGMA, A6926). Spots were counted by ImmunoSpot reader (Cellular Technology Limited)

Computational Analysis

FASTQ reads were quality checked and trimmed with FastQC. High quality trimmed reads were mapped to the mm10/GRCm38.p6 reference genome with Bowtie2 v2.3.4.3. Gene counts and differential gene expression were calculated with featureCount using the latest GENCODE main annotation file and DESeq2, respectively. Geneset functional enrichment was performed with GSEA. Downstream statistics and Plot drawing were performed with R. Heatmaps were generated with GENE-E (The Broad Institute of MIT and Harvard).

Statistics

Values are expressed as mean±standard deviation as indicated. All statistical analysis was carried out in GraphPad Prism 8.0, using one-way ANOVA, two-way ANOVA, Mantel-Cox test (survival curves) and non-parametric Mann-Whitney U test (two-tailed) where unpaired t-test was applied. P-values below 0.05 were considered significant. In multi-group comparisons, multiple testing correction for pairwise tests among groups was applied using Tukey's post hoc analysis.

### Example 2

We designed an shRNA (shMecp2) targeting the 3′-UTR sequence of the mouse Mecp2 gene, which was able to downregulate Mecp2 RNA by 50 fold and able to reduce MeCP2 protein levels by 70% in mouse primary neurons after 7 days from the transduction with an shRNA-expressing lentivirus (FIG. 13).

We then generated an AAV vector (shMecp2-iMecp2) with both the iMecp2 construct and the shMecp2 cassette. This AAV was produced in AAV-PHP.eB viral particles and wild-type mouse primary cortical neurons were transduced with a viral concentration of 1×10¹⁰ vg/ml. 7 days after transduction, the neurons were lysed and Mecp2 protein levels were assessed by Western blotting.

Interestingly, with this vector MeCP2 levels remained comparable to those found in wild-type neurons transduced with a vector expressing only a scrambled shRNA sequence.

(FIG. 14). These results demonstrate that the shMecp2-iMecp2-AAV system is capable of downregulating the Mecp2 endogenous gene in wild-type neurons while expressing the viral Mecp2 sequence, maintaining overall physiological levels of the protein.

Sequences of vectors used in these studies include:

AAV-shMecp2-CBA-GFP:

(SEQ ID NO: 23)
CCTGCAGGCAGCTGCGCGCTCGCTCGCTCACTGAGGCCGCCCGGGCAAA
GCCCGGGCGTCGGGCGACCTTTGGTCGCCCGGCCTCAGTGAGCGAGCGA
GCGCGCAGAGAGGGAGTGGCCAACTCCATCACTAGGGGTTCCTGCGGCC
TCTAGAGAACGCTGACGTCATCAACCCGCTCCAAGGAATCGCGGGCCCA
GTGTCACTAGGCGGGAACACCCAGCGCGCGTGCGCCCTGGCAGGAAGAT
GGCTGTGAGGGACAGGGGAGTGGCGCCCTGCAATATTTGCATGTCGCTA
TGTGTTCTGGGAAATCACCATAAACGTGAAATGTCTTTGGATTTGGGAA
TCTTATAAGTTCTGTATGAGACCACAGATCCCCGATTGTAGATTCAGGT
TAATTCAAGAGATTAACCTGAATCTACAATCTTTTTGGAAAAGCTTATC
GATAACGCGCTAGTTATTAATAGTAATCAATTACGGGGTCATTAGTTCA
TAGCCCATATATGGAGTTCCGCGTTACATAACTTACGGTAAATGGCCCG
CCTGGCTGACCGCCCAACGACCCCCGCCCATTGACGTCAATAATGACGT
ATGTTCCCATAGTAACGCCAATAGGGACTTTCCATTGACGTCAATGGGT
GGAGTATTTACGGTAAACTGCCCACTTGGCAGTACATCAAGTGTATCAT
ATGCCAAGTACGCCCCCTATTGACGTCAATGACGGTAAATGGCCCGCCT
GGCATTATGCCCAGTACATGACCTTATGGGACTTTCCTACTTGGCAGTA
CATCTACGTATTAGTCATCGCTATTACCATGGTCGAGGTGAGCCCCACG
TTCTGCTTCACTCTCCCCATCTCCCCCCCCTCCCCACCCCCAATTTTGT
ATTTATTTATTTTTTAATTATTTTGTGCAGCGATGGGGGCGGGGGGGGG
GGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAA
AGTTTCCTTTTATGGCGAGGCGGCGGCGGCGGCGGCCCTATAAAAAGCG
AAGCGCGCGGCGGGCGGGGAGTCGCTGCGACGCTGCCTTCGCCCCGTGC
CCCGCTCCGCCGCCGCCTCGCGCCGCCCGCCCCGGCTCTGACTGACCGC
GTTACTCCCACAGGTGAGCGGGCGGGACGGCCCTTCTCCTCCGGGCTGT
AATTAGCCCGTTTAGTGAACCGTCAGATCGCCTGGAGACGCCATCCACG
CTGTTTTGACCTCCATAGAAGACACCGGGACCGATCCAGCCTCCGCGGA
TTCGAATCCCGGCCGGGAACGGTGCATTGGAACGCGGATTCCCCGTGCC
AAGAGTGACGTAAGTACCGCCTATAGAGTCTATAGGCCCACAAAAAATG
CTTTCTTCTTTTAATATACTTTTTTGTTTATCTTATTTCTAATACTTTC
CCTAATCTCTTTCTTTCAGGGCAATAATGATACAATGTATCATGCCTCT
TTGCACCATTCTAAAGAATAACAGTGATAATTTCTGGGTTAAGGCAATA
GCAATATTTCTGCATATAAATATTTCTGCATATAAATTGTAACTGATGT
AAGAGGTTTCATATTGCTAATAGCAGCTACAATCCAGCTACCATTCTGC
TTTTATTTTATGGTTGGGATAAGGCTGGATTATTCTGAGTCCAAGCTAG
GCCCTTTTGCTAATCATGTTCATACCTCTTATCTTCCTCCCACAGCTCC
TGGGCAACGTGCTGGTCTGTGTGCTGGCCCATCACTTTGGCAAAGAATT
GGGATTCGAACACGGTCGACGAATTCGTTAACGGATCCGAACGCCACCA
TGggcaagcctatccctaaccctctgctgggcctggactccacaGGCAG
CGGCACCGGTGGATCCTCTAGAatggtgagcaagggcgaggagctgttc
accggggtggtgcccatcctggtcgagctggacggcgacgtaaacggcc
acaagttcagcgtgtccggcgagggcgagggcgatgccacctacggcaa
gctgaccctgaagttcatctgcaccaccggcaagctgcccgtgccctgg
cccaccctcgtgaccaccctgacctacggcgtgcagtgcttcagccgct
accccgaccacatgaagcagcacgacttcttcaagtccgccatgcccga
aggctacgtccaggagcgcaccatcttcttcaaggacgacggcaactac
aagacccgcgccgaggtgaagttcgagggcgacaccctggtgaaccgca
tcgagctgaagggcatcgacttcaaggaggacggcaacatcctggggca
caagctggagtacaactacaacagccacaacgtctatatcatggccgac
aagcagaagaacggcatcaaggtgaacttcaagatccgccacaacatcg
aggacggcagcgtgcagctcgccgaccactaccagcagaacacccccat
cggcgacggccccgtgctgctgcccgacaaccactacctgagcacccag
tccgccctgagcaaagaccccaacgagaagcgcgatcacatggtcctgc
tggagttcgtgaccgccgccgggatcactctcggcatggacgagctgta
caagtaaGCTAGAGATATCCTCGAGAGATCGATCTACGGGTGGCATCCC
TGTGACCCCTCCCCAGTGCCTCTCCTGGCCCTGGAAGTTGCCACTCCAG
TGCCCACCAGCCTTGTCCTAATAAAATTAAGTTGCATCATTTTGTCTGA
CTAGGTGTCCTTCTATAATATTATGGGGTGGAGGGGGGTGGTATGGAGC
AAGGGGCAAGTTGGGAAGACAACCTGTAGGGCCTGCGGGGTCTATTGGG
AACCAAGCTGGAGTGCAGTGGCACAATCTTGGCTCACTGCAATCTCCGC
CTCCTGGGTTCAAGCGATTCTCCTGCCTCAGCCTCCCGAGTTGTTGGGA
TTCCAGGCATGCATGACCAGGCTCAGCTAATTTTTGTTTTTTTGGTAGA
GACGGGGTTTCACCATATTGGCCAGGCTGGTCTCCAACTCCTAATCTCA
GGTGATCTACCCACCTTGGCCTCCCAAATTGCTGGGATTACAGGCGTGA
ACCACTGCTCCCTTCCCTGTCCTTCTGATTTTGTAGGTAACCACGTGCG
GACCGAGCGGCCGCAGGAACCCCTAGTGATGGAGTTGGCCACTCCCTCT
CTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCTG
CCTGCAGG

Legend:

Bold—shRNA sequence

Lower case, not underlined—GFP sequence

Bold and underlined—ITR AAV sequence

Italic and underlined—sequence of H1 promotor

Underlined—sequence of CBA promotor

Lower case and underlined—tag V5 sequence

Italic—hGH polyA sequence

AAV-shMecp2-iMecp2:

CCTGCAGGCAGCTGCGCGCTCGCTCGCTCACTGAGGCCGCCCGGGCAAAGCCCGGGCGTCGGGCGACCTTT
GGTCGCCCGGCCTCAGTGAGCGAGCGAGCGCGCAGAGAGGGAGTGGCCAACTCCATCACTAGGGGTTCCTG
CGGCCTCTAGAGAACGCTGACGTCATCAACCCGCTCCAAGGAATCGCGGGCCCAGTGTCACTAGGCGGGAA
CACCCAGCGCGCGTGCGCCCTGGCAGGAAGATGGCTGTGAGGGACAGGGGAGTGGCGCCCTGCAATATTTG
CATGTCGCTATGTGTTCTGGGAAATCACCATAAACGTGAAATGTCTTTGGATTTGGGAATCTTATAAGTTC
TGTATGAGACCACAGATCCCCGATTGTAGATTCAGGTTAATTCAAGAGATTAACCTGAATCTACAATCTTT
TTGGAAAAGCTTATCGATAACGCGCTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTACGCCCCCTATTGAC
GTCAATGACGGTAAATGGCCCGCCTGGCATTATGCCCAGTACATGACCTTATGGGACTTTCCTACTTGGCA
GTACATCTACGTATTAGTCATCGCTATTACCATGGTCGAGGTGAGCCCCACGTTCTGCTTCACTCTCCCCA
TCTCCCCCCCCTCCCCACCCCCAATTTTGTATTTATTTATTTTTTAATTATTTTGTGCAGCGATGGGGGCG
GGGGGGGGGGGGGGGCGCGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGCGGCGGCGGCGGCGGC
CCTATAAAAAGCGAAGCGCGCGGCGGGCGGGGAGTCGCTGCGACGCTGCCTTCGCCCCGTGCCCCGCTCCG
CCGCCGCCTCGCGCCGCCCGCCCCGGCTCTGACTGACCGCGTTACTCCCACAGGTGAGCGGGCGGGACGGC
CCTTCTCCTCCGGGCTGTAATTAGCCCGTTTAGTGAACCGTCAGATCGCCTGGAGACGCCATCCACGCTGT
TTTGACCTCCATAGAAGACACCGGGACCGATCCAGCCTCCGCGGATTCGAATCCCGGCCGGGAACGGTGCA
TTGGAACGCGGATTCCCCGTGCCAAGAGTGACGTAAGTACCGCCTATAGAGTCTATAGGCCCACAAAAAAT
GCTTTCTTCTTTTAATATACTTTTTTGTTTATCTTATTTCTAATACTTTCCCTAATCTCTTTCTTTCAGGG
CAATAATGATACAATGTATCATGCCTCTTTGCACCATTCTAAAGAATAACAGTGATAATTTCTGGGTTAAG
GCAATAGCAATATTTCTGCATATAAATATTTCTGCATATAAATTGTAACTGATGTAAGAGGTTTCATATTG
CTAATAGCAGCTACAATCCAGCTACCATTCTGCTTTTATTTTATGGTTGGGATAAGGCTGGATTATTCTGA
GTCCAAGCTAGGCCCTTTTGCTAATCATGTTCATACCTCTTATCTTCCTCCCACAGCTCCTGGGCAACGTG
CTGGTCTGTGTGCTGGCCCATCACTTTGGCAAAGAATTGGGATTCGAACACGGTCGACGAATTCGTTAACG
GATCCGAACGCCACCATGggcaagcctatccctaaccctctgctgggcctggactccacaGGCAGCGGCAC
CGGTatggccgccgctgccgccaccgccgccgccgccgccgcgccgagcggaggaggaggaggaggcgagg
aggagagactggaggaaaagtcagaagaccaggatctccagggcctcagagacaagccactgaagtttaag
aaggcgaagaaagacaagaaggaggacaaagaaggcaagcatgagccactacaaccttcagcccaccattc
tgcagagccagcagaggcaggcaaagcagaaacatcagaaagctcaggctctgccccagcagtgccagaag
cctcggcttcccccaaacagcggcgctccattatccgtgaccggggacctatgtatgatgaccccaccttg
cctgaaggttggacacgaaagcttaaacaaaggaagtctggccgatctgctggaaagtatgatgtatattt
gatcaatccccagggaaaagcttttcgctctaaagtagaattgattgcatactttgaaaaggtgggagaca
cctccttggaccctaatgattttgacttcacggtaactgggagagggagcccctccaggagagagcagaaa
ccacctaagaagcccaaatctcccaaagctccaggaactggcaggggtcggggacgccccaaagggagcgg
cactgggagaccaaaggcagcagcatcagaaggtgttcaggtgaaaagggtcctggagaagagccctggga
aacttgttgtcaagatgcctttccaagcatcgcctgggggtaagggtgagggaggtggggctaccacatct
gcccaggtcatggtgatcaaacgccctggcagaaagcgaaaagctgaagctgacccccaggccattcctaa
gaaacggggtagaaagcctgggagtgtggtggcagctgctgcagctgaggccaaaaagaaagccgtgaagg
agtcttccatacggtctgtgcatgagactgtgctccccatcaagaagcgcaagacccgggagacggtcagc
atcgaggtcaaggaagtggtgaagcccctgctggtgtccacccttggtgagaaaagcgggaagggactgaa
gacctgcaagagccctgggcgtaaaagcaaggagagcagccccaaggggcgcagcagcagtgcctcctccc
cacctaagaaggagcaccatcatcaccaccatcactcagagtccacaaaggcccccatgccactgctccca
tccccacccccacctgagcctgagagctctgaggaccccatcagcccccctgagcctcaggacttgagcag
cagcatctgcaaagaagagaagatgccccgaggaggctcactggaaagcgatggctgccccaaggagccag
ctaagactcagcctatggtcgccaccactaccacagttgcagaaaagtacaaacaccgaggggagggagag
cgcaaagacattgtttcatcttccatgccaaggccaaacagagaggagcctgtggacagccggacgcccgt
gaccgagagagttagctga

[symbol]

[symbol]

[symbol]

[symbol]

[symbol]

[symbol]

[symbol]

[symbol]

[symbol]

[symbol]

[symbol]

[symbol]

[symbol]

GATATCTCTAGAGATATCCTCGAGAGATCTACGGGTGGCATCCCTGTGACCCCTCCCCAGTGCCTCTCCTG
GCCCTGGAAGTTGCCACTCCAGTGCCCACCAGCCTTGTCCTAATAAAATTAAGTTGCATCATTTTGTCTGA
CTAGGTGTCCTTCTATAATATTATGGGGTGGAGGGGGGTGGTATGGAGCAAGGGGCAAGTTGGGAAGACAA
CCTGTAGGGCCTGCGGGGTCTATTGGGAACCAAGCTGGAGTGCAGTGGCACAATCTTGGCTCACTGCAATC
TCCGCCTCCTGGGTTCAAGCGATTCTCCTGCCTCAGCCTCCCGAGTTGTTGGGATTCCAGGCATGCATGAC
CAGGCTCAGCTAATTTTTGTTTTTTTGGTAGAGACGGGGTTTCACCATATTGGCCAGGCTGGTCTCCAACT
CCTAATCTCAGGTGATCTACCCACCTTGGCCTCCCAAATTGCTGGGATTACAGGCGTGAACCACTGCTCCC
TTCCCTGTCCTTCTGATTTTGTAGGTAACCACGTGCGGACCGAGCGGCCGCAGGAACCCCTAGTGATGGAG
TTGGCCACTCCCTCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGCTGCCTGCAGG

Legend:

Bold—shRNA sequence

Lower case, not underlined—iMecp2 sequence

Bold and underlined—ITR AAV sequence

Italic and underlined—sequence of H1 promotor

Underlined—sequence of CBA promotor

Lower case and underlined—tag V5 sequence

Bold, italic and underlined—3′UTR Mecp2 sequence

Italic—hGH polyA sequence

All publications mentioned in the above specification are herein incorporated by reference. Various modifications and variations of the disclosed agents, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A polynucleotide comprising a nucleotide sequence encoding methyl-CpG binding-protein 2 (MeCP2) operably linked to a strong promoter and a 3′-UTR, wherein the 3′-UTR is less than or equal to about 1000 bp in length.

2. The polynucleotide of claim 1, wherein the nucleotide sequence encoding MeCP2 comprises a sequence selected from the group consisting of:
(a) a nucleotide sequence encoding an amino acid sequence that has at least 70% identity to SEQ ID NO: 1 or 2;
(b) a nucleotide sequence that has at least 70% identity to SEQ ID NO: 3 or 4; and
(c) the nucleotide sequence of SEQ ID NO: 3 or 4.

3. The polynucleotide of claim 1, wherein the promoter is a chicken β-actin (CBA) promoter.

4. The polynucleotide of claim 1, wherein the 3′-UTR is less than or equal to about 500 bp in length.

5. The polynucleotide of claim 1, wherein the polynucleotide further comprises a polyadenylation sequence operably linked to the nucleotide sequence encoding MeCP2.

6. The polynucleotide of claim 1, wherein the polynucleotide further comprises a nucleotide sequence encoding an inhibitor of MeCP2 expression.

7. The polynucleotide of claim 6, wherein the inhibitor is an shRNA, siRNA, miRNA or antisense DNA/RNA.

8. A vector comprising the polynucleotide of claim 1.

9. The vector of claim 8, wherein the vector is a viral vector.

10. The vector of claim 8, wherein the vector is in the form of a viral vector particle.

11. The vector of claim 10, wherein the viral vector particle is an AAV vector particle that comprises a capsid selected from the group consisting of an AAV9; AAV9 PHP.B; AAV9 PHP.eB; and AAVrh10 capsid.

12. A cell comprising the polynucleotide of claim 1.

13. A pharmaceutical composition comprising the polynucleotide of claim 1 and a pharmaceutically-acceptable carrier, diluent or excipient.

14. The pharmaceutical composition of claim 13, formulated for fa) systemic or local delivery; and/or (b) intravascular, intravenous, intra-arterial, intracranial or intraparenchymal brain delivery.

15-22. (canceled)

23. The vector of claim 8, wherein the vector is an AAV, retroviral, lentiviral or adenoviral vector.

24. A method for treating or preventing Rett syndrome comprising administering the vector of claim 8 to a subject in need thereof.

25. The method of claim 24, wherein the vector is administered to a subject: (a) systemically or locally; and/or (b) intracranially or intraparenchymally.

26. The method of claim 24, wherein the vector is administered simultaneously, sequentially or separately in combination with an immunosuppressant.

27. The method of claim 24, wherein the vector is administered at a dosage of 108 to 1012 vg/20 g.

28. The method of claim 24, wherein the vector is administered at a dosage of 5×109 to 5×1014 vg per kg.
